(19) United States
(12) Patent Application Publication
(10) Pub. No.: US 2025/0186463 A1
(43) Pub. Date: Jun. 12, 2025

(54) ABIRATERONE DECANOATE PRODRUGS AND USE IN THERAPY

(71) Applicant: PROPELLA THERAPEUTICS, INC., Pittsboro, NC (US)

(72) Inventors: Matthew J. SHARP, Apex, NC (US); William R. MOORE, JR., Pittsboro, NC (US)

(21) Appl. No.: 18/842,725

(22) PCT Filed: Feb. 15, 2023
(86) PCT No.: PCT/US2023/013116

§ 371 Date: Aug. 29, 2024

Related U.S. Application Data
(60) Provisional application No. 63/315,240, filed on Mar. 1, 2022.
(60) Provisional application No. 63/425,839, filed on Nov. 16, 2022.

(51) Int. Cl. A61K 31/58; A61K 9/00; A61K 31/4166; A61K 31/573; A61K 45/06; A61K 47/10; A61K 47/14; A61K 47/44; A61P 35/00

(52) U.S. Cl. CPC A61K 31/58; A61K 9/0019; A61K 31/4166; A61K 31/573; A61K 45/06; A61K 47/10; A61K 47/14; A61K 47/44; A61P 35/00

(57) ABSTRACT

Sustained-release abiraterone prodrug formulations, methods, and kits for parenteral administration to a human subject having a sex hormone-dependent or androgen receptor driven disease or disorder, such as a sex hormone-dependent benign or malignant disorder such as prostate cancer, an androgen receptor driven cancer, and/or a syndrome due to androgen excess.

This application claims the benefit of U.S. Provisional Application Nos. 63/315,240, filed Mar. 1, 2022, and 63/425,839, filed Nov. 16, 2022, the content of each of which is incorporated herein by reference in its entirety.

The present disclosure relates generally to novel prodrugs of abiraterone and long-acting, depot-based parenteral formulations of abiraterone prodrugs. The disclosure is subject to a wide range of applications, such as for intramuscular (IM) injection to a patient suffering from a sex hormone-dependent or androgen receptor driven disease or disorder, such as an androgen or estrogen hormone-dependent benign or malignant disorder or androgen receptor driven cancer, including various cancers (such as prostate cancer, bladder cancer, hepatocellular carcinoma, lung cancer, breast cancer, and ovarian cancer, etc.), and for the treatment of non-oncologic syndromes due to the overproduction of androgens (including both classical and nonclassical congenital adrenal hyperplasia, endometriosis, polycystic ovary syndrome, precocious puberty, hirsutism, etc.).

### BACKGROUND

Abiraterone ((3β)-17-(pyridin-3-yl) androsta-5, 16-dien-3-ol; CAS #: 154229-19-3); Formula: C₂₄H₃₁NO; Mol. Weight: 349.5 g/mol) is an inhibitor of CYP17A1 (which is a member of the cytochrome P450 superfamily of enzymes that catalyze the synthesis of cholesterol, steroids and other lipids and are involved in drug metabolism). CYP17A1 has both 17α-hydroxylase activity and 17,20-lyase activity. Abiraterone potently and selectively inhibits both CYP17A1 17α-hydroxylase and 17,20-lyase enzyme activities, hereinafter may be simply referred to as CYP17A1 hydroxylase activity and CYP17A1 lyase activity, or just hydroxylase or lyase activity, respectively. The 17α-hydroxylase activity of CYP17A1 is required for the generation of glucocorticoids such as cortisol. However, both the hydroxylase and 17,20-lyase activities of CYP17A1 are required for the production of androgenic (e.g., androstenedione, testosterone, and dihydrotestosterone) and estrogenic (estrone, estradiol, estratriol) steroids through the conversion of 17α-hydroxypregnenolone to the sex steroid precursor, dehydroepiandrosterone, see FIG. 1. Thus, abiraterone interferes with the synthesis of androgens and estrogens in the gonads (primarily in the testes and ovaries) and extra-gonadally (e.g., in the adrenals and in the tumors themselves).

Though abiraterone itself is poorly absorbed, it can be administered orally as an abiraterone acetate prodrug. Abiraterone acetate is also poorly absorbed, but can be converted to abiraterone in the gut, which is poorly absorbed into the bloodstream following the cleavage of the acetate prodrug. Abiraterone acetate ((3)-17-(3-Pyridyl)androsta-5, acetate; CAS #154229-18-2) is approved in the United States for treatment of castration resistant or castration sensitive prostate cancer under the brand name Zytiga®. Abiraterone acetate is now also available globally.

It is known that orally administered abiraterone acetate prodrug is not significantly absorbed by the gastrointestinal tract (and little prodrug can be detected in blood plasma). Instead, it has been shown that abiraterone acetate is hydrolyzed to abiraterone in the intraluminal environment resulting in generation of abiraterone supersaturation, which is responsible for creating the strong driving force for abiraterone absorption (Stappaerts et al., Eur. J. Pharmaceutics Biopharmaceutics 90:1, 2015).

Because abiraterone blocks the normal physiologic production of steroids by the adrenal glands, its prodrug formulation is commonly prescribed with administration of a low dose of a steroid to prevent adrenal insufficiency. Indeed, Zytiga® (250 mg tablets) is approved in the United States only in combination with prednisone for the treatment of patients with metastatic castration resistant prostate cancer (CRPC) and patients with metastatic castration-sensitive prostate cancer (CSPC). The prescribing information provided with Zytiga® recommends oral administration of 1,000 mg (4×250 mg tablets) once daily in combination with prednisone (5 mg) administered orally twice daily for CRPC patients or once daily for CSPC patients. In Europe, the use of Zytiga® is approved only in combination with either prednisone or prednisolone.

Because the administration of abiraterone acetate with food increases the absorption of abiraterone acetate and, therefore, has the potential to result in increased and highly variable exposures, which can potentially cause various side effects including cardiovascular side effects and/or hepatotoxicity etc., the prodrug should be consumed on an empty stomach at least one hour before, or two hours after, a meal. Indeed, the prescribing information for Zytiga® states it must be taken on an empty stomach, and no food should be consumed for at least two hours before oral dosing and at least one hour after oral dosing.

The prescribing information explains that for a daily oral dose of 1,000 mg of Zytiga® in patients with metastatic CRPC, abiraterone's steady-state Cₘₐₓ values were 226±178 ng/mL (mean±SD) and its area under the curve (AUC) values were 1173±690 ng·hr/mL (mean±SD). A single-dose (1,000 mg) cross-over study of Zytiga® in healthy subjects found that systemic exposure of abiraterone was increased when Zytiga® was administered with food. Specifically, abiraterone's Cₘₐₓ and AUC values were approximately 7- and 5-fold higher, respectively, when Zytiga® was administered with a low-fat meal (7% fat, 300 calories) and approximately 17- and 10-fold higher, respectively, when Zytiga® was administered with a high-fat meal (57% fat, 825 calories).

The currently approved solid dosage oral form of the prodrug abiraterone acetate has several disadvantages. For example, it has very low bioavailability that necessitates a large daily pill burden for patients (4×250 mg tablets once daily). In addition, it causes highly variable blood levels in patients due to the combination of low bioavailability and a large food effect. Further, as abiraterone is rapidly cleared, this approved dosing regimen results in a daily Cₘᵢₙ of abiraterone, which is believed to be associated with a loss of therapeutic effect in metastatic CRPC patients. In addition, it was known that abiraterone acetate is a non-selective CYP17A1 inhibitor and inhibits both hydroxylase and lyase activities, leading to the undesired increase in hydroxylase upstream steroids. For example, it was known that oral administration of abiraterone acetate can cause increased level of progesterone, which can activate certain progesterone-activated mutant androgen receptors and lead to resistance to CYP17A1 inhibition. See e.g., Chen, E. J. et al. Clin. Cancer. Res. 21(6):1273-1280 (2014).

Non-oral modes of administration (for example, parenteral routes) have been explored for other classes of drugs. However, to date, there are no marketed sustained-release injectable prodrug formulations of abiraterone.

### SUMMARY

The present disclosure generally relates to novel abiraterone prodrugs, long-acting abiraterone prodrug formulations, and methods of using the same, for example, in treating a human subject having a sex hormone-dependent or androgen receptor driven disease or disorder, such as a sex hormone-dependent benign or malignant disorder, an androgen receptor driven cancer, and/or a syndrome due to androgen excess, see also U.S. Pat. No. 10,792,292 B2, issued to Propella Therapeutics, Inc. on Oct. 6, 2020; PCT Application Nos. PCT/US2021/048607, filed Sep. 1, 2021, and PCT/US2022/016278, filed Feb. 14, 2022, the content of each of which is herein incorporated by reference in its entirety.

The novel abiraterone prodrugs can typically be a fatty acid ester of abiraterone, which upon cleavage, releases abiraterone and a safe and degradable fatty acid component. Compared to oral abiraterone acetate formulation, the novel abiraterone prodrugs and formulations herein are a breakthrough in that they provide increased bioavailability, elimination of the food effect, reduced pill burden, less frequent dosing frequency, and sustained effective blood plasma levels of abiraterone, for example, for at least one week, typically, for at least two weeks and up to ten weeks or more following administration of the abiraterone prodrug formulation. Further, pharmacokinetics and pharmacodynamics studies of representative abiraterone prodrugs demonstrate that the novel abiraterone prodrugs and formulations are suitable for dosing once a week, once a month, once every two months, once every three months, or even less frequently, for treating a human subject having a sex hormone-dependent or androgen receptor driven disease or disorder, such as a sex hormone-dependent benign or malignant disorder, an androgen receptor driven cancer, and/or a syndrome due to androgen excess. This feature alone represents a significant improvement over the currently marketed Zytiga® tablets, which require a large daily pill burden for patients (4×250 mg tablets once daily).

As detailed herein, the present disclosure shows that administering abiraterone prodrugs, in particular abiraterone decanoate, can selectively inhibit CYP17A1 lyase activity over that of CYP17A1 hydroxylase activity in human subjects, which provides new treatment options where lyase selectivity is desired or deemed beneficial. In particular, as shown in the Examples section and figures herein, human subjects treated with intramuscular injection(s) of abiraterone decanoate can achieve lyase selectivity, without a significant increase of steroid levels that can occur due to inhibition of CYP17A1 hydroxylase activities. Further, the clinical results herein show that human subjects treated with abiraterone decanoate did not experience mineralocorticoid toxicities, which are common side effects associated with administering Zytiga® tablets to human subjects. See e.g., McKay, R. R. et al. Cancer 125(4):524-532 (2019). The lyase selectivity found in human following administering abiraterone decanoate was unexpected; the same selectivity was not observed through administering Zytiga® tablets to human subjects. For example, it was known that oral administration of abiraterone acetate can lead to 80-fold increases in concentrations of mineralocorticoids which can lead to significant side effects, 30% of men receiving abiraterone acetate have signs and symptoms of mineralocorticoid excess—hypertension, hypokalemia, and edema, and likely produce resistance to the regimen as 40-fold increases in progesterone and pregnenolone produced from the oral abiraterone acetate regimen can stimulate androgen receptor, progesterone receptor, etc. See e.g., Wright, C., et al. Eur, J. Endocrinol. 182(4):413-421 (2020) and Zytiga® Prescribing Information (2011). On the other hand, FIG. 11 shows that the fold changes from baseline for corticosterone and progesterone observed from cohorts 3, 4, and 5 are significantly reduced in comparison to those changes reported in the literature for Zytiga at Day 28. Additionally, the present disclosure shows that administering abiraterone prodrugs, in particular abiraterone decanoate, is effective in reducing or eliminating cancers metastasized to one or more lymph nodes. Without wishing to be bound by theories, it is believed that the observed efficacy in treating cancers metastasized to lymph nodes is due in part to the lymphatic delivery of agents (for example, lipophilic prodrugs such as abiraterone decanoate), which results in higher concentrations of active drugs within the lymph system.

The present disclosure also shows that administering abiraterone prodrugs, in particular abiraterone decanoate, can consistently reduce PSA levels in patients throughout the treatment period (see e.g., FIG. 7). Further, the response rate (in terms of reduction of PSA levels) in CRPC patients is also much higher observed in the study described herein when compared to the literature reported response rate for abiraterone acetate and enzalutamide, see e.g., FIGS. 9A and 9B.

The present disclosure also shows that certain pharmaceutically acceptable antioxidants are advantageous in stabilizing abiraterone prodrug, in particular, abiraterone decanoate formulations for room temperature storage.

Further, the present disclosure shows that administering abiraterone prodrugs, in particular abiraterone decanoate, can be effective in treating patients whose disease (cancer) have progressed after androgen receptor antagonist based treatment, in particular, enzalutamide treatment. In some embodiments, the abiraterone prodrugs herein can also be used in combination with androgen receptor antagonists, such as enzalutamide, to achieve a synergistic effect in suppressing cancer growth.

The present disclosure also shows that administering abiraterone decanoate to human subjects are generally well tolerated. This is consistent with earlier observations in animal studies, in which no liver toxicity was observed from intramuscular administration of abiraterone decanoate at the tested doses.

Thus, the compositions and methods described herein provide new treatment options in human subjects that cannot be achieved by the marketed oral formulation Zytiga® tablets. In addition, the compositions and methods described herein fulfill a long-felt and unmet need by providing an alternative to oral formulations that suffer from (1) low bioavailability, (2) interactions with ingested food, (3) delivery of highly variable blood levels of parent drug with the possibility of reduced efficacy and increased side effects, (4) requirement of daily dosing and high pill burden, (5) requirement of castration, (6) poor patient compliance due to required abstinence from food within hours of administration, high pill burden, and the need for complementary daily administration of prednisone or prednisolone with a conflicting dosing schedule as it is to be taken with food, and (7) undesired mineralocorticoid excess, in particular, progesterone.

In some embodiments, the present disclosure provides the following embodiments in enumerated paragraphs [1]-[109]:

[1]A method of treating a disease or disorder in a human subject in need thereof, the method comprising administering, preferably, parenterally administering, to the human subject a therapeutically effective amount of a pharmaceutical composition comprising an abiraterone prodrug (e.g., an abiraterone lipophilic ester), wherein the disease or disorder is sex hormone-dependent or androgen receptor driven, such as a sex hormone-dependent benign or malignant disorder or a syndrome due to androgen excess.

[2] The method of [1], wherein the abiraterone prodrug is abiraterone decanoate, or a pharmaceutically acceptable salt thereof,

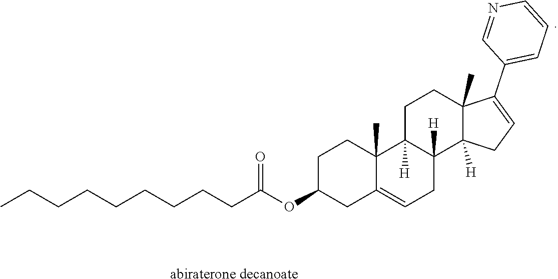

[3] The method of [1] or [2], wherein the method selectively inhibits CYP17A1 lyase activity over CYP17A1 hydroxylase activity in the human subject.

[4] The method of any of [1]-[3], wherein the disease or disorder is polycystic ovary syndrome (PCOS), congenital adrenal hyperplasia (CAH), or endometriosis.

[5] The method of any of [1]-[3], wherein the disease or disorder is a cancer, wherein the cancer is a sex hormone dependent or androgen receptor driven cancer.

[6] The method of [5], wherein the cancer is prostate cancer, endometrial cancer, or ovarian cancer.

[7] The method of [5], wherein the cancer is prostate cancer.

[8] The method of any of [5]-[7], wherein the cancer has metastasized to one or more lymph nodes, and the administering of the pharmaceutical composition is effective in inhibiting growth of the cancer in the one or more lymph nodes.

[9] The method of any of [5]-[8], wherein the cancer is a prostate cancer characterized with a Gleason score of ≤6.

[10] The method of any of [5]-[9], wherein the human subject has a measurable prostate specific antigen.

[11] The method of any of [5]-[7], wherein the cancer is a localized prostate cancer, e.g., a high risk localized prostate cancer.

[12] The method of any of [5]-[7], wherein the cancer is a metastatic castration-sensitive prostate cancer, non-metastatic castration-sensitive prostate cancer, non-metastatic castration-resistant prostate cancer, or metastatic castration-resistant prostate cancer.

[13] The method of any of [5]-[7], wherein the cancer is a newly diagnosed high risk metastatic hormone sensitive prostate cancer.

[14] The method of any of [5]-[7], wherein the cancer is a metastatic CRPC (mCRPC), wherein the human subject is asymptomatic or mildly symptomatic after failure of androgen deprivation therapy in whom chemotherapy is not yet clinically indicated.

[15] The method of any of [5]-[7], wherein the cancer is a metastatic CRPC (mCRPC), wherein the human subject's disease has progressed on or after a taxane-based chemotherapy regimen, such as docetaxel-based or cabazitaxel-based chemotherapy regimen.

[16] The method of any of [5]-[7], wherein the cancer is a refractory prostate cancer.

[17] The method of any of [5]-[16], wherein (i) the human subject's disease has progressed on or after an androgen receptor antagonist based treatment, such as enzalutamide based treatment; and/or (ii) the human subject has developed resistance to the treatment of abiraterone acetate in combination with prednisone, including resistance due to increased levels of progesterone.

[18] The method of any of [1]-[17], wherein the pharmaceutical composition comprises abiraterone decanoate in its basic form and a pharmaceutically acceptable carrier.

[19] The method of [18], wherein the pharmaceutically acceptable carrier comprises a pharmaceutically acceptable oil and optionally a further pharmaceutically acceptable solvent.

[20] The method of [19], wherein the pharmaceutically acceptable oil comprises a triglyceride (e.g., long and/or medium chain triglycerides), and the further pharmaceutically acceptable solvent, if present, comprises an alcohol, ester, and/or acid solvent.

[21] The method of [19] or [20], wherein the pharmaceutically acceptable oil is selected from vegetable oil, castor oil, corn oil, sesame oil, cottonseed oil, peanut oil, poppy seed oil, tea seed oil, and soybean oil, and the further pharmaceutically acceptable solvent, if present, comprises benzyl alcohol, benzyl benzoate, or a combination thereof.

[22] The method of any one of [18]-[21], wherein the pharmaceutically acceptable carrier comprises corn oil, benzyl alcohol, and benzyl benzoate.

[23] The method of any one of [1]-[22], wherein the pharmaceutical composition comprises, for each milliliter, (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); and (d) corn oil, q.s. to 1 milliliter.

[24] The method of any one of [1]-[22], wherein the pharmaceutical composition comprises, for each milliliter, (a) abiraterone decanoate in its basic form, in an amount of about 180 mg; (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); and (d) corn oil, q.s. to 1 milliliter.

[25] The method of any one of [18]-[24], wherein the weight ratio of benzyl alcohol to benzyl benzoate in the pharmaceutical composition ranges from about 2:1 to about 1:5 (e.g., about 1:1 to 1:3, such as about 1:2).

[26] The method of any one of [1]-[25], wherein the abiraterone prodrug is abiraterone decanoate, and the abiraterone decanoate is substantially pure, e.g., characterized as having a purity by weight of at least 95%, preferably, at least 98%, such as about 98.5%, about 99%, about 99.5%, or higher.

[27] The method of any one of [1]-[26], wherein the pharmaceutical composition is characterized as having a viscosity of less than 0.1 Pa*s, such as about 0.05 Pa*s or lower.

[28] The method of any one of [1]-[27], wherein the pharmaceutical composition is characterized as having a glide force of about 1-10 N when measured using a 21G, 1.5 inch needle, and/or about 2-15 N when measured using a 23G, 1.5 inch needle, and/or about 30-150 N when measured using a 27G, 1.5 inch needle.

[29] The method of any one of [1]-[28], wherein the pharmaceutical composition is characterized as having no more than 1000 particles having a size of 10 m or greater, and no more than 300 particles having a size of 25 μm or greater, when measured according to USP <788> and/or <789>.

[30] The method of any one of [1]-[29], wherein the pharmaceutical composition is characterized as having less than 100 EU/ml, such as less than 25 EU/ml of bacterial endotoxins measured according to USP <85>.

[31] The method of any one of [1]-[30], wherein the human subject is non-castrated.

[32] The method of any one of [1]-[30], wherein the human subject is castrated.

[33] The method of any one of [1]-[30] and [32], wherein the human subject is treated with a gonadotropin-releasing hormone agonist and/or antagonist.

[34] The method of any one of [1]-[33], wherein the pharmaceutical composition is administered to the human subject through an intramuscular injection.

[35] The method of any one of [1]-[34], wherein the pharmaceutical composition is administered to the human subject once every 1-3 months, such as once every three months.

[36] The method of any one of [1]-[35], wherein each administration of the pharmaceutical composition comprises administering to the human subject about 50 mg to about 2000 mg of abiraterone decanoate, such as about 180 mg, about 360 mg, about 720 mg, about 1260 mg, about 1800 mg, or any range between the recited values, preferably, the pharmaceutical composition is administered to the human subject once every three months and each administration of the pharmaceutical composition comprises administering to the human subject about 1260 mg of abiraterone decanoate.

[37] The method of any one of [1]-[36], further comprising administering to the human subject a 1^{st}-generation androgen receptor antagonist, e.g., proxalutamide, bicalutamide, flutamide, nilutamide, topilutamide.

[38] The method of any one of [1]-[37], further comprising administering to the human subject a 2^{nd}-generation androgen receptor antagonist (e.g., apalutamide, darolutamide or enzalutamide).

[39] The method of [38], wherein the 2^{nd}-generation androgen receptor antagonist is enzalutamide.

[40] The method of any one of [1]-[39], further comprising administering to the human subject a 3^{rd} generation androgen receptor antagonist (such as an N-terminal domain inhibitor) or an androgen receptor degrader molecule, alone or in combination with one or more 1^{st} generation or 2^{nd} generation androgen receptor antagonists.

[41] The method of any one of [1]-[40], further comprising administering to the human subject one or more agents selected from hydrocortisone, prednisone, prednisolone, methylprednisolone, and dexamethasone, preferably, dexamethasone.

[42] The method of any one of [1]-[41], further comprising administering to the human subject a poly ADP ribose polymerase (PARP) inhibitor, e.g., niraparib, rucaparib, olaparib, talazoparib, veliparib, and fluzoparib.

[43] The method of any one of [1]-[42], further comprising administering to the human subject a chemotherapeutic agent, such as a taxane based chemotherapeutic agent (e.g., docetaxel, cabazitaxel, paclitaxel, etc.) or platinum based chemotherapeutic agent (e.g., cisplatin, carboplatin, oxaliplatin, etc.).

[44] The method of any one of [1]-[43], further comprising administering to the human subject an immunotherapy, such as administering Sipuleucel-T, an immune checkpoint inhibitor (e.g., anti-PD-1 antibody such as pembrolizumab or nivolumab, or anti-PD-L1 antibody such as avelumab or atezolizumab), or an anti-CTLA-4 antibody (e.g., ipilimumab), etc.

[45] The method of any one of [1]-[44], further comprising administering to the human subject a bispecific T-cell engager (BiTE) therapy, such as blinatumomab or solitomab.

[46] The method of any one of [1]-[45], further comprising administering to the human subject a kinase inhibitor, e.g., sunitinib, dasatinib, cabozantinib, erdafitinib, dovitinib, capivasertib, onvansertib, ipatasertib, afuresertib, alisertib, apitolisib, opaganib, etc.

[47] The method of any one of [1]-[46], further comprising administering to the human subject a bone protecting agent (e.g., denosumab, zolendronic acid), and wherein the human subject is characterized as having prostate cancer (e.g., CRPC) with bone metastasis.

[48] The method of any one of [1]-[47], further comprising administering to the human subject a therapeutic agent selected from 1) an anti-IL23 targeting monoclonal antibody, e.g., tildrakizumab; 2) a selenium, such as sodium selenite; 3) an EZH2 inhibitor, e.g., CPI-1205, GSK2816126, or tazemetostat; 4) a CDK4/6 inhibitor, e.g., palbociclib, ribociclib, abemaciclib; 6) a bromodomain and extra-terminal domain (BET) inhibitor, e.g., CCS1477, INCB057643, alobresib, ZEN-3694, or molibresib (GSK525762); 7) an anti-CD105 antibody, e.g., TRC105 or carotuximab; 8) niclosamide; 9) an A2A receptor antagonist, e.g., AZD4635; 10) a PI3K inhibitor, e.g., AZD-8186, buparlisib, or dactolisib; 11) a further non-steroidal CYP17A1 inhibitor, e.g. seviteronel; 12) an antiprogestogen, e.g., onapristone; 13) navitoclax; 14) an HSP90 inhibitor, e.g., onalespib (AT13387); 15) an HSP27 inhibitor, e.g., OGX-427; 16) a 5-alpha-reductase inhibitor, e.g., dutasteride; 17) metformin; 18) AMG-386; 19) dextromethorphan; 20) theophylline; 21) hydroxychloroquine; and 22) lenalidomide.

[49] The method of any one of [1]-[48], further comprising administering to the human subject one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof.

[50] The method of any one of [1]-[49], wherein (i) the human subject is chemotherapy naïve or hormone therapy naïve prior to being administered the pharmaceutical composition, such as a chemotherapy naïve mCRPC patient; and/or (ii) wherein the human subject suffers from hepatic impairment, such as moderate to severe hepatic impairment (Child-Pugh Class B or C), prior to being administered the pharmaceutical composition.

[51] The method of any one of [1]-[50], wherein (i) the human subject has prostate cancer, and the method does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition; and/or (ii) the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition.

[52] The method of any one of [1]-[51], wherein the human subject suffers from one or more side effects associated with inhibition of CYP17A1 hydroxylase activity or the human subject is susceptible to one or more side effects associated with inhibition of CYP17A1 hydroxylase activity, such as mineralocorticoid toxicities.

[53] The method of any one of [1]-[52], wherein the abiraterone prodrug is abiraterone decanoate, and at least a portion of the administered abiraterone decanoate is absorbed through the lymphatic system.

[54] The method of any one of [1]-[53], wherein the administering of the pharmaceutical composition is effective in achieving a sustained reduction of serum testosterone level in the human subject to 50% below baseline or lower within 15 days of the first administration of the pharmaceutical composition.

[55] The method of [54], wherein the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition.

[56] The method of any one of [1]-[55], wherein the administering of the pharmaceutical composition does not enhance serum progesterone level in the human subject (i) by more than 40% above baseline at 4 weeks following the first administration of the pharmaceutical composition; and/or (ii) by more than 40% above baseline from 2 weeks to 12 weeks following the second administration of the pharmaceutical composition.

[57] The method of any one of [1]-[55], wherein the administering of the pharmaceutical composition does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the pharmaceutical composition.

[58] The method of any one of [1]-[57], wherein as applicable, the administering of the pharmaceutical composition reduces the level of prostate specific antigen in the human subject, preferably, the level of prostate specific antigen is reduced to 50% or below, preferably, 90% or below, compared to the baseline PSA level, at least at one time point following the first administration of the pharmaceutical composition and/or following one or more subsequent administration of the pharmaceutical composition.

[59]A method of reducing serum testosterone level in a human subject in need thereof, the method comprising parenterally administering to the human subject an effective amount of a pharmaceutical composition comprising an abiraterone prodrug (e.g., an abiraterone lipophilic ester), wherein the pharmaceutical composition is administered in an effective amount to achieve a sustained reduction of serum testosterone level in the human subject to 50% below baseline or lower within 15 days of the first administration of the pharmaceutical composition, wherein the administering of the pharmaceutical composition does not enhance serum progesterone level in the human subject (i) by more than 40% above baseline at 4 weeks following the first administration of the pharmaceutical composition; and/or (ii) by more than 40% above baseline from 2 weeks to 12 weeks following the second administration of the pharmaceutical composition, preferably, does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the pharmaceutical composition.

[60] The method of [59], wherein the abiraterone prodrug is abiraterone decanoate, or a pharmaceutically acceptable salt thereof,

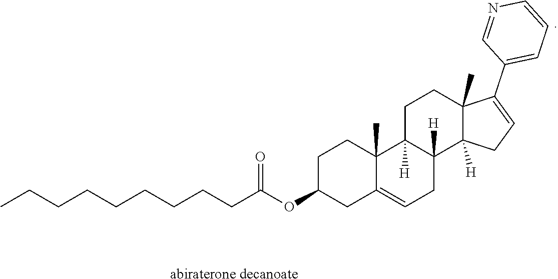

[61] The method of [59] or [60], wherein the human subject suffers from a disease or disorder that is sex hormone-dependent or androgen receptor driven, such as a sex hormone-dependent benign or malignant disorder or a syndrome due to androgen excess.

[62] The method of [61], wherein the disease or disorder is any of those described in [4]-[17].

[63] The method of any of [59]-[62], wherein the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition.

[64] The method of [59]-[63], wherein the human subject suffers from one or more side effects associated with inhibition of CYP17A1 hydroxylase activity or the human subject is susceptible to one or more side effects associated with inhibition of CYP17A1 hydroxylase activity, such as mineralocorticoid toxicities.

[65]A method of treating a cancer in a human subject in need thereof, wherein the cancer is a sex hormone dependent or androgen receptor driven cancer, which has metastasized to one or more lymph nodes, the method comprising parenterally administering to the human subject a therapeutically effective amount of a pharmaceutical composition comprising an abirateronc prodrug (e.g., an abirateronc lipophilic ester).

[66] The method of [65], wherein the abiraterone prodrug is abiraterone decanoate, or a pharmaceutically acceptable salt thereof,

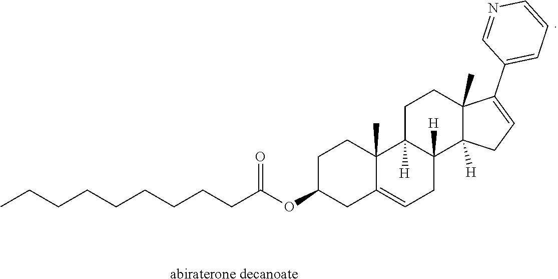

[67] The method of [65] or [66], wherein the administering of the pharmaceutical composition is effective in inhibiting growth of the cancer in the one or more lymph nodes.

[68] The method of any of [65]-[67], wherein the cancer is prostate cancer, e.g., any of those described in [8]-[17] as applicable.

[69]A method of treating a cancer in a human subject in need thereof, wherein the cancer is a sex hormone dependent or androgen receptor driven cancer, wherein the human subject's disease has progressed on or after an androgen receptor antagonist based treatment, such as enzalutamide based treatment, the method comprising parenterally administering to the human subject a therapeutically effective amount of a pharmaceutical composition comprising an abiraterone prodrug (e.g., an abiraterone lipophilic ester).

[70] The method of [69], wherein the abiraterone prodrug is abiraterone decanoate, or a pharmaceutically acceptable salt thereof,

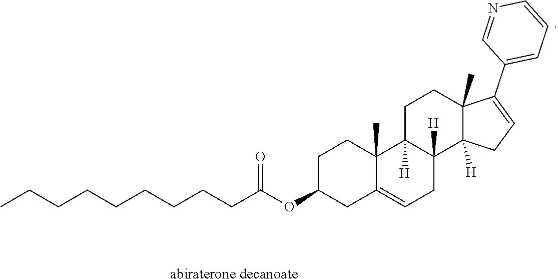

[71] The method of [69] or [70], wherein the cancer is prostate cancer, e.g., any of those described in [8]-[17] as applicable.

[72]A method of treating a cancer in a human subject in need thereof, wherein the cancer is a sex hormone dependent or androgen receptor driven cancer, the method comprising parenterally administering to the human subject a therapeutically effective amount of a pharmaceutical composition comprising an abiraterone prodrug (e.g., an abiraterone lipophilic ester), wherein the human subject is further treated with an androgen receptor antagonist, such as enzalutamide, prior to, concurrent, or subsequent to the first parenteral administration of the pharmaceutical composition.

[73] The method of [72], wherein the abiraterone prodrug is abiraterone decanoate, or a pharmaceutically acceptable salt thereof,

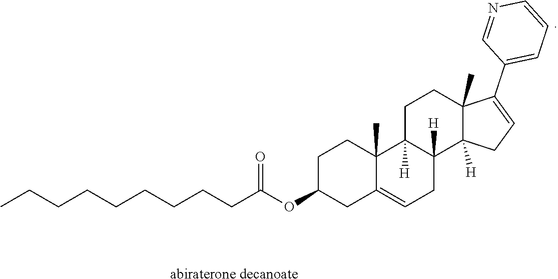

[74] The method of [72] or [73], wherein the cancer is prostate cancer, e.g., any of those described in [8]-[17] as applicable.

[75] The method of any of [59]-[74], wherein the pharmaceutical composition is as described in any of [18]-[30].

[76] The method of any of [59]-[75], wherein the pharmaceutical composition is administered to the human subject through an intramuscular injection.

[77] The method of any of [59]-[76], wherein the pharmaceutical composition is administered to the human subject once every 1-3 months, such as once every three months.

[78] The method of any of [59]-[77], wherein each administration of the pharmaceutical composition comprises administering to the human subject about 50 mg to about 2000 mg of abiraterone decanoate, such as about 180 mg, about 360 mg, about 720 mg, about 1260 mg, about 1800 mg, or any range between the recited values, preferably, the pharmaceutical composition is administered to the human subject once every three months and each administration of the pharmaceutical composition comprises administering to the human subject about 1260 mg of abiraterone decanoate.

[79] The method of any of [59]-[78], wherein the human subject is castrated.

[80] The method of any one of [59]-[79], wherein the human subject is treated with a gonadotropin-releasing hormone agonist and/or antagonist.

[81] The method of any of [59]-[78], wherein the human subject is non-castrated.

[82] The method of any of [59]-[81], wherein (i) the human subject is chemotherapy naïve or hormone therapy naïve prior to being administered the pharmaceutical composition, such as a chemotherapy naïve mCRPC patient; and/or (ii) wherein the human subject suffers from hepatic impairment, such as moderate to severe hepatic impairment (Child-Pugh Class B or C), prior to being administered the pharmaceutical composition.

[83] The method of any of [59]-[82], wherein (i) the human subject has prostate cancer, and the method does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition; and/or (ii) the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition.

[84] The method of any one of [65]-[83], wherein the human subject suffers from one or more side effects associated with inhibition of CYP17A1 hydroxylase activity or the human subject is susceptible to one or more side effects associated with inhibition of CYP17A1 hydroxylase activity, such as mineralocorticoid toxicities.

[85] The method of any one of [59]-[84], wherein the abiraterone prodrug is abiraterone decanoate, and at least a portion of the administered abiraterone decanoate is absorbed through the lymphatic system.

[86] The method of any of [59]-[85], further comprising administering to the human subject one or more additional therapy according to any of [37]-[49].

[87] The method of any of [1]-[86], wherein the abiraterone prodrug is administered in an effective amount to reduce the serum testosterone level in the human subject to 80% below baseline or lower, such as 85% below baseline or lower, or 90% below baseline or lower, when measured at 24 weeks after the first administration of the abiraterone prodrug.

[88] The method of any of any of [1]-[87], wherein the abiraterone prodrug is administered in an effective amount to reduce the serum testosterone level in the human subject to about 50 ng/dL or below (e.g., about 40 ng/dL or below, about 30 ng/dL or below, about 20 ng/dL or below, about 10 ng/dL or below, etc.), when the human subject is a non-castrated human subject, or about 1 ng/dL or below, when the human subject is a castrated human subject, when measured at 24 weeks after the first administration of the abiraterone prodrug.

[89] The method of any of [1]-[88], wherein the human subject would benefit from selective inhibition of CYP17A1 lyase activity over CYP17A1 hydroxylase activity.

[90] The method of any of [1]-[40] and [42]-[89], as applicable, wherein the administering of the pharmaceutical composition does not cause a mineralocorticoid toxicity in the human subject.

[91] The method of any of [1]-[40] and [42]-[90], as applicable, wherein the human subject is not treated with an agent effective for treating a mineralocorticoid toxicity.

[92] The method of any of [1]-[40] and [42]-[90], as applicable, wherein the human subject is not treated with an agent that is a glucocorticoid or a mineralocorticoid receptor antagonist.

[93]A pharmaceutical composition comprising an abiraterone decanoate solution, wherein each milliliter of the abiraterone decanoate solution comprises: (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 20 mg (e.g., about 0.5 mg, about 1 mg, about 2 mg, or about 5 mg); and (c) corn oil, q.s. to 1 milliliter, wherein the abiraterone decanoate has the structure of:

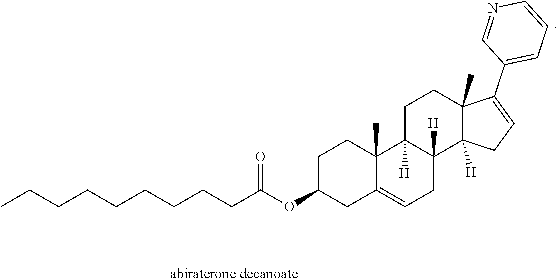

[94] The pharmaceutical composition of [93], wherein each milliliter of the abiraterone decanoate solution comprises: (a) abiraterone decanoate in its basic form, in an amount of about 180 mg; (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 2 mg (e.g., about 0.5 mg, about 1 mg, or about 2 mg); and (e) corn oil, q.s. to 1 milliliter.

[95] The pharmaceutical composition of [93] or [94], wherein the weight ratio of benzyl alcohol to benzyl benzoate in the pharmaceutical composition ranges from about 2:1 to about 1:5 (e.g., about 1:1 to 1:3, such as about 1:2).

[96] The pharmaceutical composition of any one of [93]-[95], wherein the abiraterone decanoate is substantially pure, e.g., characterized as having a purity by weight of at least 95%, preferably, at least 98%, such as about 98.5%, about 99%, about 99.5%, or higher.

[97] The pharmaceutical composition of any one of [93]-[96], wherein the abiraterone decanoate solution is included in a container having a headspace, e.g., a vial, an ampule, a bottle, etc., wherein the headspace is filled with an inert gas, such as nitrogen gas.

[98] The pharmaceutical composition of any one of [93]-[96], wherein the abiraterone decanoate solution is included in a syringe.

[99] The pharmaceutical composition of any one of [93]-[98], which is storage stable at room temperature, for 1 month or longer, preferably, for 2 months or 3 months, or longer.

[100] The pharmaceutical composition of any one of [93]-[99], wherein the abiraterone decanoate solution is characterized as having a viscosity of less than 0.1 Pa*s, such as about 0.05 Pa*s or lower.

[101] The pharmaceutical composition of any one of [93]-[100], wherein the abiraterone decanoate solution is characterized as having a glide force of about 1-10 N when measured using a 21G, 1.5 inch needle, and/or about 2-15 N when measured using a 23G, 1.5 inch needle, and/or about 30-150 N when measured using a 27G, 1.5 inch needle.

[102] The pharmaceutical composition of any one of [93]-[101], wherein the abiraterone decanoate solution is characterized as having no more than 1000 particles having a size of 10 μm or greater, and no more than 300 particles having a size of 25 m or greater, when measured according to USP <788> and/or <789>.

[103] The pharmaceutical composition of any one of [93]-[102], wherein the abiraterone decanoate solution is characterized as having less than 100 EU/ml, such as less than 25 EU/ml of bacterial endotoxins measured according to USP <85>.

[104] The pharmaceutical composition of any one of [93]-[103], wherein the pharmaceutical composition is in the form of a unit dosage form, wherein the unit dosage form comprises about 1 ml to about 20 ml of the abiraterone decanoate solution.

[105] The pharmaceutical composition of any one of [93]-[103], wherein the pharmaceutical composition is in the form of a unit dosage form, wherein the unit dosage form comprises about 1 or 2 ml of the abiraterone decanoate solution.

[106] The pharmaceutical composition of any one of [93]-[103], wherein the pharmaceutical composition is in the form of a unit dosage form, wherein the unit dosage form comprises about 3 ml of the abiraterone decanoate solution.

[107] The pharmaceutical composition of any one of [93]-[103], wherein the pharmaceutical composition is in the form of a unit dosage form, wherein the unit dosage form comprises about 4 ml to about 10 ml (e.g., about 4, 5, 6, 7, 8, 9, or 10 mL) of the abiraterone decanoate solution, preferably, about 7 ml of the abiraterone decanoate solution.

[108] The method of any of [1]-[92], wherein the pharmaceutical composition, as applicable, is any of those according to [93]-[107].

[109] The method of any of [1]-[90] and [108], comprising administering to the human subject (i) abiraterone decanoate intramuscularly once every one to three months, preferably, once every three months, preferably, about 1260 mg of abiraterone decanoate once every three months; and (ii) dexamethasone orally once daily, preferably, at a daily dose of about 0.5 mg/day.

Embodiments of the present disclosure can fulfill a long felt need in the field of sex hormone-dependent disorders and oncology including the treatment of a sex hormone dependent or androgen receptor driven cancer such as prostate cancer. Embodiments of the present disclosure can also fulfill a long felt need in the field of treating syndromes due to androgen excess syndrome. Embodiments of the present disclosure can overcome major disadvantages and deficiencies of prior art formulations (including commercially-available oral dosage forms) of abiraterone acetate, by providing long-acting, sustained release depot-based parenteral formulations of abiraterone prodrugs, methods of producing the same, methods of treatment using the same, and kits for convenient administration of the formulations to subjects in need of therapy for various disorders including prostate cancer.

There has thus been outlined, rather broadly, features in order that the detailed description thereof that follows may be better understood, and in order that the present contribution to the art may be better appreciated. There are, of course, additional features that will be described further hereinafter. Indeed, it is to be understood that both the foregoing general description and the following detailed description are exemplary and explanatory and are intended to provide further explanation of the disclosure.

In this respect, before explaining at least one embodiment in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those persons skilled in the art will appreciate that the conception upon which this disclosure is based can readily be utilized as a basis for the designing of other formulations, methods, systems, kits, and compositions for carrying out the several purposes of the present disclosure. It is important, therefore, that equivalent constructions insofar as they do not depart from the spirit and scope of the present disclosure, are included in the present disclosure.

The accompanying drawings are included to provide a further understanding and are incorporated in and constitute a part of this specification, illustrate several embodiments, and together with the description explain the principles.

### BRIEF DESCRIPTION OF THE DRAWINGS

FIG. 1 presents biochemical pathways showing the effects of CYP17A1 inhibition on the synthesis of androgens, estrogens, glucocorticoids, progesterone, and mineralocorticoids.

FIG. 2 shows a representative analysis of a batch of high purity abiraterone decanoate.

FIG. 3A shows results of serum testosterone level changes from baseline (cycle 1, day 1 value or C1D1 value) following intramuscular injections of 180 mg, 360 mg, 720 mg, 1260 mg, or 1800 mg of abiraterone decanoate to patients in cohorts 1, 2, 3, 4, and 5, respectively. Cohorts 1 and 2 were also orally administered prednisone, 5 mg once daily or twice daily; cohorts 3, 4, and 5 were orally administered dexamethasone, 0.5 mg once daily; cohorts 1, 2, 3, 4, and 5 in other figures were treated with the same glucocorticoid regimen, respectively.

FIG. 3B shows results of serum dehydroepiandrosterone sulfate (DHEA-S) level changes from baseline (cycle 1, day 1 value) following intramuscular injections of 180 mg, 360 mg, 720 mg, 1260 mg, or 1800 mg of abiraterone decanoate to patients in cohorts 1, 2, 3, 4, and 5, respectively.

FIG. 3C shows results of serum dehydroepiandrosterone (DHEA) level changes from baseline (cycle 1, day 1 value) following intramuscular injections of 180 mg, 360 mg, 720 mg, 1260 mg, or 1800 mg of abiraterone decanoate to patients in cohorts 1, 2, 3, 4, and 5, respectively.

FIG. 3D shows results of serum androstenedione level changes from baseline (cycle 1, day 1 value) following intramuscular injections of 180 mg, 360 mg, 720 mg, 1260 mg, or 1800 mg of abiraterone decanoate to patients in cohorts 1, 2, 3, 4, and 5, respectively.

FIG. 3E shows results of serum progesterone level changes from baseline (cycle 1, day 1 value) following intramuscular injections of 180 mg, 360 mg, 720 mg, 1260 mg, or 1800 mg of abiraterone decanoate to patients in cohorts 1, 2, 3, 4, and 5, respectively.

FIG. 3F shows results of serum 11-deoxycorticosterone level changes from baseline (cycle 1, day 1 value) following intramuscular injections of 720 mg, 1260 mg, or 1800 mg of abiraterone decanoate to patients in cohorts 3, 4, and 5, respectively.

FIG. 3G shows results of serum corticosterone level changes from baseline (cycle 1, day 1 value) following intramuscular injections of 720 mg, 1260 mg, or 1800 mg of abiraterone decanoate to patients in cohorts 3, 4, and 5, respectively.

FIG. 4A shows plasma abiraterone decanoate concentration vs. time profile following intramuscular injections of 180 mg, 360 mg, 720 mg, 1260 mg, or 1800 mg of abiraterone decanoate to patients in cohorts 1, 2, 3, 4, and 5, respectively.

FIG. 4B shows plasma abiraterone concentration vs. time profile following intramuscular injections of 180 mg, 360 mg, 720 mg, 1260 mg, or 1800 mg of abiraterone decanoate to patients in cohorts 1, 2, 3, 4, and 5, respectively.

FIG. 4C shows plasma abiraterone sulfate concentration vs. time profile following intramuscular injections of 180 mg, 360 mg, 720 mg, 1260 mg, or 1800 mg of abiraterone decanoate to patients in cohorts 1, 2, 3, 4, and 5, respectively.

FIG. 4D shows plasma abiraterone N-oxide concentration vs. time profile following intramuscular injections of 180 mg, 360 mg, 720 mg, 1260 mg, or 1800 mg of abiraterone decanoate to patients in cohorts 1, 2, 3, 4, and 5, respectively.

FIG. 4E shows plasma N-oxide abiraterone sulfate concentration vs. time profile following intramuscular injections of 180 mg, 360 mg, 720 mg, 1260 mg, or 1800 mg of abiraterone decanoate to patients in cohorts 1, 2, 3, 4, and 5, respectively.

FIG. 5 shows serum steroid levels changes compared to baseline following intramuscular injection of 180 mg of abiraterone decanoate to patient 1.

FIG. 6 shows serum steroid levels changes compared to baseline following intramuscular injection of 360 mg of abiraterone decanoate to patient 2.

FIG. 7 shows patient swim plots against prostate specific antigen (PSA) responses (% change from baseline) during the study period for cohorts 3, 4, and 5.

FIG. 8A shows best PSA responses (% change from baseline) during the study period for all patients in cohorts 3, 4, and 5.

FIG. 8B shows best PSA responses (% change from baseline) during the study period for pre-chemo CRPC patients in cohorts 3, 4, and 5.

FIG. 9A shows a comparison of percentage of patients who achieved ≥50% decline in PSA from baseline at any point in study from Chemo-naïve CRPC patients of cohorts 3, 4, and 5 with literature reported value obtained from abiraterone acetate (AA)/Prednisone or enzalutamide (Enza). Results for pre-chemo CRPC patients. PRL-02 represents i.m. abiraterone decanoate treatment.

FIG. 9B shows a comparison of percentage of patients who achieved ≥90% decline in PSA from baseline at any point in study from Chemo-naïve CRPC patients of cohorts 3, 4, and 5 with literature reported value obtained from abiraterone acetate (AA) or enzalutamide (Enza). Results for pre-chemo CRPC patients.

FIG. 10 shows a plot of percentage changes of PSA from baseline during the study period for all patients studied with CRPC.

FIG. 11A shows a bar graph comparing the fold increase from baseline of mean mineralocorticoid in Cohort 3, 4 or 5 vs. Zytiga/Prednisone.

FIG. 11B shows the serum progesterone level vs. time profile for patients in Cohorts 4 and 5, with the LLOQ being 1.59 nM. Maximum individual progesterone level for Cohorts 1-3 is 2.5 nM.

FIG. 12 shows a representative flow diagram illustrating the steps typically performed in the manufacturing process for Abiraterone Decanoate Solution.

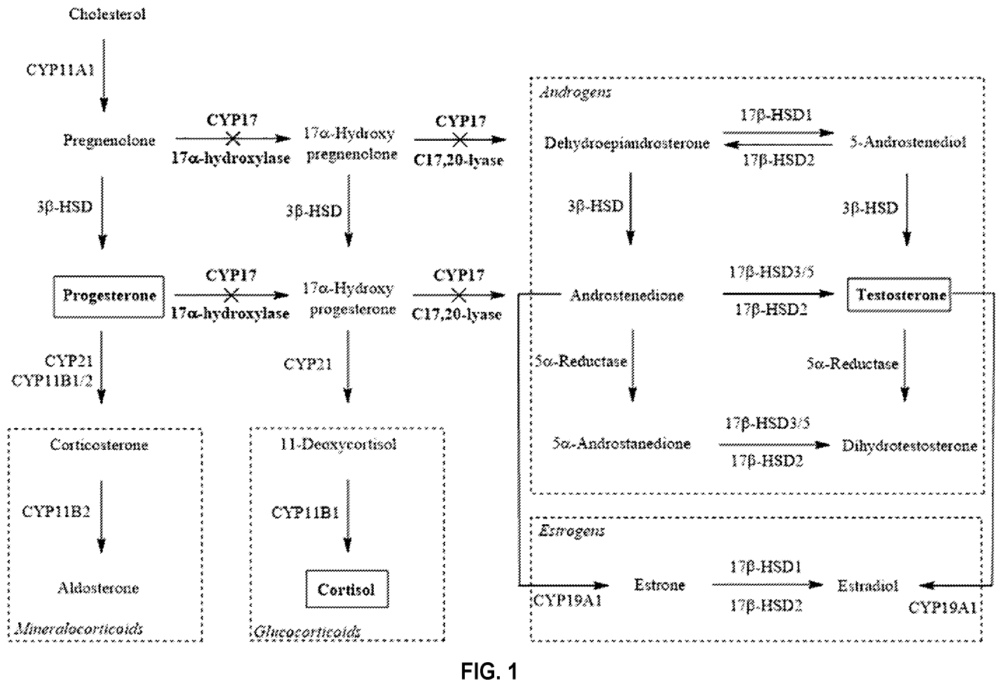

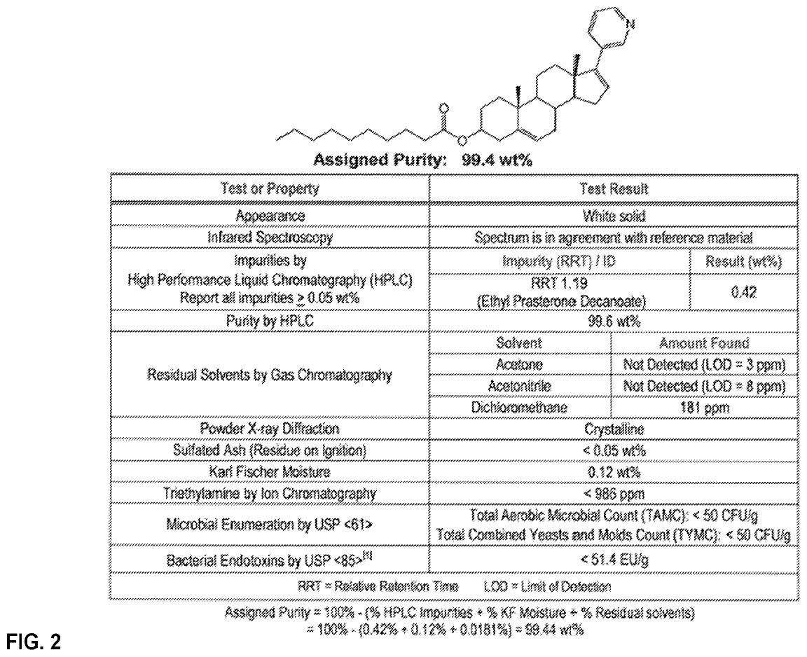

### DETAILED DESCRIPTION

In a broad aspect, the present disclosure relates to novel methods for treating or preventing a disease or disorder associated with sex hormones. Some embodiments of the present disclosure are based, in part, on the unexpected discovery that abiraterone prodrugs can be administered to a human subject to selectively inhibit CYP17A1 lyase activity over hydroxylase activity. Some embodiments of the present disclosure are also based, in part, on the unexpected discovery that administering an abiraterone prodrug to a human subject can reduce or eliminate cancers metastasized to one or more lymph nodes. Some embodiments of the present disclosure are based, in part, on the unexpected discovery that administering an abiraterone prodrug can be effective in treating a human subject who has progressed on or after an androgen receptor antagonist based treatment, such as enzalutamide based treatment. Data shown in the present disclosure further suggests that the abiraterone prodrugs herein can achieve synergistic effects when used in combination with an androgen receptor antagonist, such as enzalutamide. Some embodiments of the present disclosure are based in part on Applicant's finding that the inclusion of a pharmaceutically acceptable antioxidant, in particular, monothioglycerol, in abiraterone decanoate formulation can lead to a storage stable formulation at room temperature for 9 months or more.

As detailed herein and described in part in U.S. Pat. No. 10,792,292 B2, and PCT Application Nos. PCT/US2021/048607 and PCT/US2022/016278, the method herein can be further advantageous over existing methods in many aspects, including but not limited to a fast and sustained reduction of serum testosterone, no need for castration, reduced or no liver toxicity compared to methods using oral abiraterone acetate formulations, improved bioavailability, elimination of the food effect associated with oral abiraterone acetate formulation, reduced pill burden, better patient compliance, decreased dosing frequency, sustained stable blood levels of active drug, reduced Cₘₐₓ, which can reduce associated side effects, etc.

Without wishing to be bound by theories, it is also believed that formulations and methods using abiraterone decanoate herein provide distinct advantages over oral abiraterone acetate regimen in that 1) abiraterone decanoate is either actively taken up or is highly permeable to cells and tissues delivering higher concentrations of abiraterone equivalents to tissues; 2) abiraterone decanoate efficacy is therefore not dependent on plasma levels, in fact abiraterone decanoate may be initially delivered to the tissues and the plasma concentrations may be secondary and due to elimination from the tissues; 3) the long pharmacological half-life of abiraterone decanoate may be a result of the slow conversion of abiraterone decanoate to abiraterone in the tissues; and 4) the high tissue concentrations of abiraterone observed from intramuscular administration of abiraterone decanoate cannot be produced by the oral regimen since the abiraterone released in the gut has low solubility and permeability (i.e., high plasma concentrations are needed to drive the concentration up in cells and tissues).

Accordingly, in various embodiments, the present disclosure provides novel methods for modulating serum steroid hormone levels, such as for reducing testosterone levels, novel methods for treating or preventing diseases or disorders mediated by or associated with such steroids, such as sex hormone dependent or androgen receptor driven cancers, and/or abiraterone prodrugs and formulations useful for the methods.

### Methods of Treatment

In some embodiments, the present disclosure provides a method of treating a disease or disorder described herein in a human subject in need thereof. The method typically comprises parenterally administering to the human subject a therapeutically effective amount of a pharmaceutical composition comprising an abiraterone prodrug (e.g., an abiraterone lipophilic ester). The disease or disorder is typically sex hormone-dependent or androgen receptor driven, such as a sex hormone-dependent benign or malignant disorder or a syndrome due to androgen excess.

Typically, the abiraterone prodrug is abiraterone decanoate, or a pharmaceutically acceptable salt thereof,

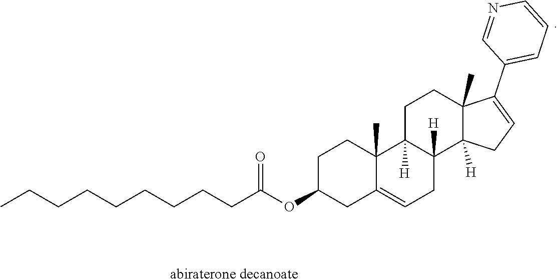

As used herein, the term “abiraterone decanoate” in its basic form should be understood as having the structure drawn above. Unless explicitly referred to as in a salt form or obviously contrary from context, “abiraterone decanoate” as used herein should be understood as in its basic form.

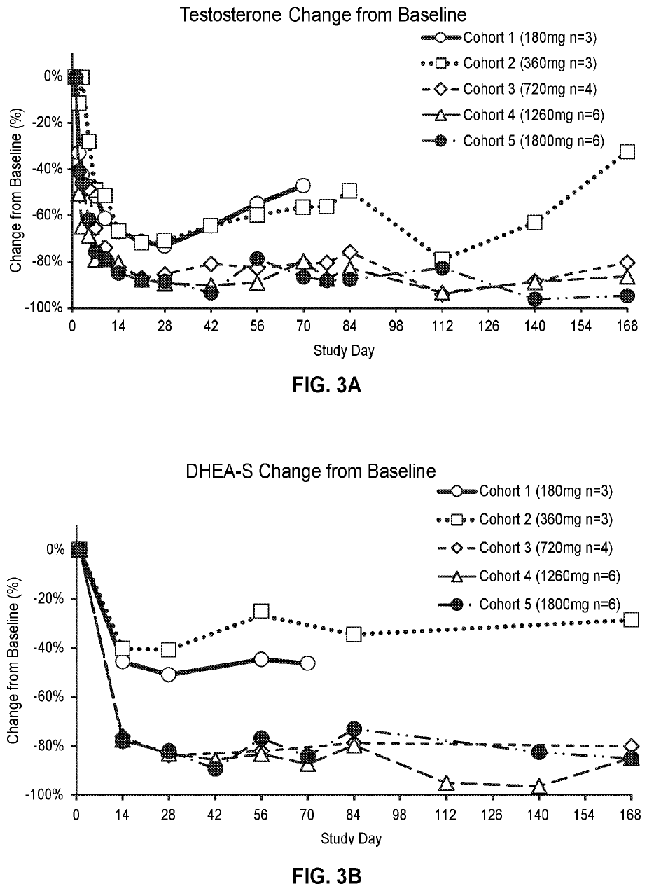

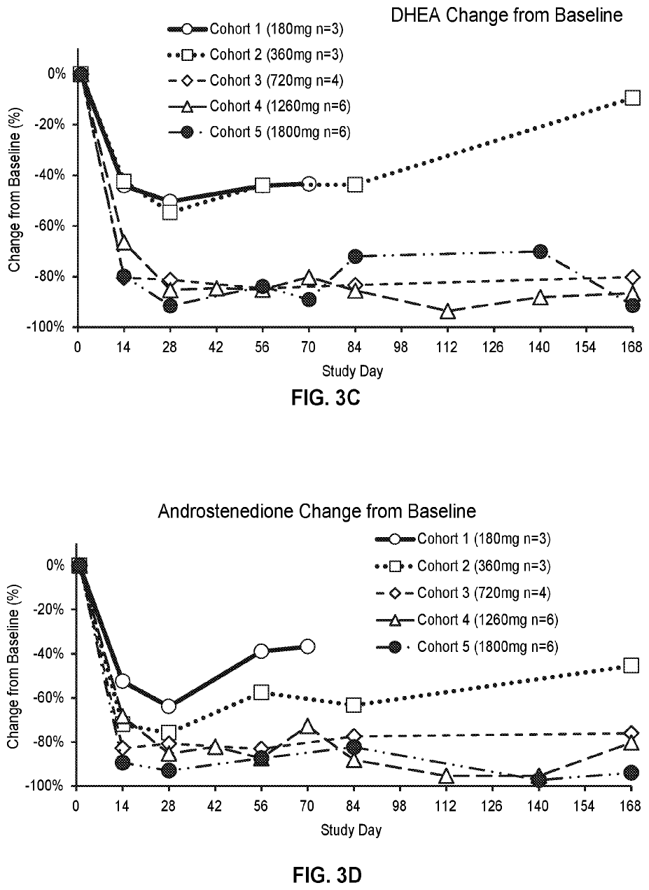

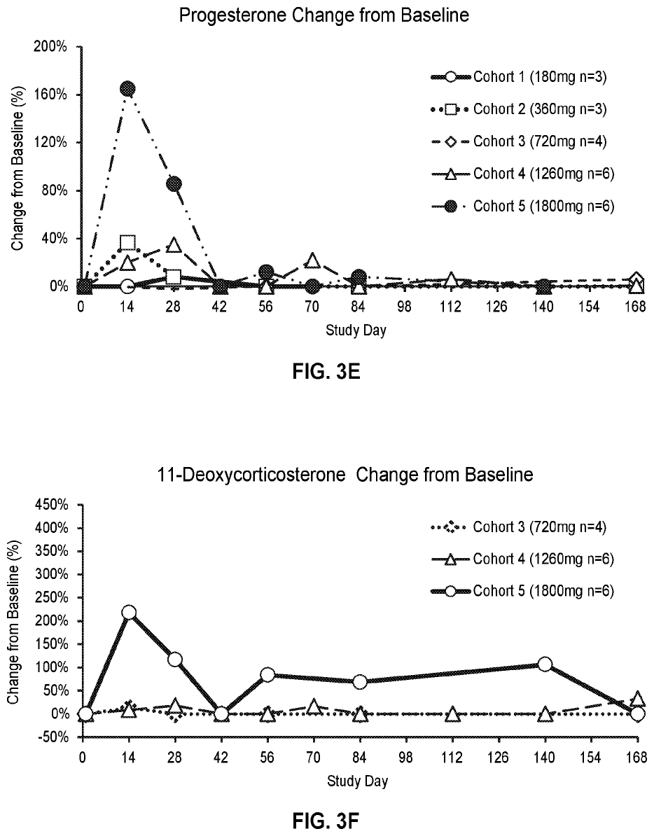

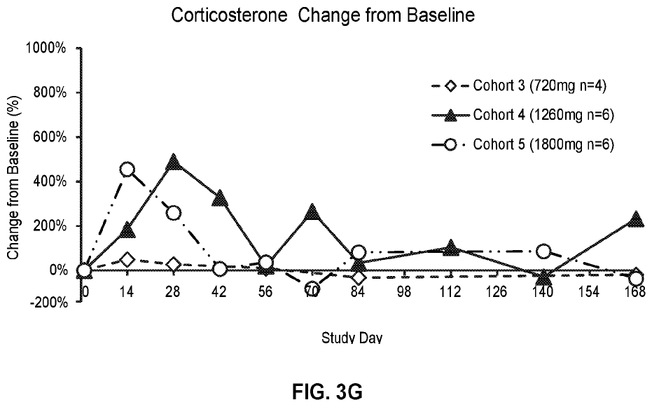

In some embodiments, the method is characterized in that the administering of the abiraterone prodrug can selectively inhibit CYP17A1 lyase activity over CYP17A1 hydroxylase activity in the human subject. See e.g., any of the methods described in [3] and [4]-[58] and [108]-[109](as applicable) of the Summary section. As shown in Example 3 herein, the present inventors unexpectedly found that administering abiraterone decanoate intramuscularly to human subjects achieved lyase selectivity, which was not shown in oral abiraterone acetate (Zytiga® tablets) treatment. The serum steroid levels show that while abiraterone decanoate administration significantly reduced the levels of androgens such as testosterone, the steroids upstream of the hydroxylase were not reduced significantly, and returned to baseline level in about 4 weeks. See e.g., FIG. 3E. As shown in FIG. 3E, even at the highest dose, the progesterone level returned to baseline in about 6 weeks, and the progesterone levels did not significantly increase during the second cycle (i.e., following the second administration of abiraterone decanoate at day 84). Thus, the method herein can be advantageously used for treating diseases or disorders in human subjects where lyase selectivity is desired or deemed beneficial, for example, endometrial cancer, endometriosis, ovarian cancer, and prostate cancer (such as localized prostate cancer, etc.). In some embodiments, the method herein can be particularly useful for treating a human subject who suffers from or is susceptible to one or more side effects associated with hydroxylase inhibition, such as from oral abiraterone acetate treatments. In some embodiments, the method herein can be particularly useful for treating a human subject who is intolerant or otherwise sensitive to increased and/or decreased steroid levels due to hydroxylase inhibition, such as increased progesterone levels. In any of the embodiments described herein, the method herein can also be particularly useful for treating a human subject who would benefit from selective inhibition of CYP17A1 lyase activity over CYP17A1 hydroxylase activity. For example, increases in progesterone (such as with a plasma progesterone level greater than 3 nM) have also been shown to be associated with poor clinical outcomes since it has been demonstrated that progesterone acts as an oncogenic hormone in prostate cancer. Hou et al. Cell Reports Medicine, 3:100561 (2022). Also, patients who respond to abiraterone treatment (e.g., Zytiga) generally relapse within 1 to 2 years. Chen et al. shows that one mechanism of such developed resistance can be associated with an increased level of progesterone, which can lead to the selection and/or activation of tumor cells expressing certain mutant androgen receptors. See e.g., Chen, E. J. et al. Clin. Cancer. Res. 2/(6):1273-1280 (2014). Thus, a prostate cancer patient who has increased level of progesterone and/or is susceptable to one or more side effects such as mineralcorticoid toxicities or drug resistance due to an increased level of progesterone, such as following oral abiraterone acetate treatment, would be beneficially treated with the methods herein. In some embodiments, the methods herein can be particularly useful for treating a human subject who has developed resistance to the treatment of oral abiraterone acetate in combination with prednisone, including resistance due to increased levels of progesterone. In some embodiments, the methods herein are for treating a human subject having prostate cancer, and the methods do not increase the level of progesterone in the human subject to a level associated with poor clinical outcomes and drug resistance. In some embodiments, because the methods herein do not cause a significant increase of progesterone levels, in such patients, a separate drug for reducing the level of progesterone is not needed to control the side effect(s) and/or drug resistance associated with the increased level of progesterone.

In some embodiments, the method herein is characterized in that it is for treating a cancer in a human subject in need thereof, wherein the cancer is a sex hormone dependent or androgen receptor driven cancer, which has metastasized to one or more lymph nodes. See e.g., any of the methods described in [65]-[68] and [75]-[92] and [108]-[109](as applicable) of the Summary section. In some embodiments, the administering of the pharmaceutical composition is effective in inhibiting growth of the cancer in the one or more lymph nodes. As shown in Example 3 herein, in at least Patient 1, administering abiraterone decanoate completely resolved the cancer metastasized to one or more lymph nodes. Without wishing to be bound by theories, it is believed that the observed efficacy in treating cancers metastasized to lymph nodes is due in part to the lymphatic delivery of agents (for example, lipophilic prodrugs such as abiraterone decanoate), which results in higher concentrations of active drugs within the lymph system. It is expected that the observed efficacy is not limited to the prostate cancer shown in the examples herein. In some embodiments, the cancer metastasized to one or more lymph nodes can be prostate cancer (e.g., as described herein).

In some embodiments, the method is characterized in that it is for treating a cancer in a human subject in need thereof, wherein the cancer is a sex hormone dependent or androgen receptor driven cancer, wherein the human subject's disease has progressed on or after an androgen receptor antagonist based treatment, such as enzalutamide based treatment. See e.g., any of the methods described in [69]-[71] and [75]-[92] and [108]-[109](as applicable) of the Summary section. In some embodiments, the cancer is prostate cancer (e.g., as described herein). As also shown in Example 3 herein, in at least Patient 2, whose disease (prostate cancer) has progressed after enzalutamide treatment, administering abiraterone decanoate was found to be also effective. This and other results herein suggest that the abiraterone prodrugs herein, in particular, abiraterone decanoate, can achieve synergistic effects when used in combination with an androgen receptor antagonist, such as enzalutamide.

In some embodiments, the method is characterized in that it is for treating a cancer in a human subject in need thereof, wherein the cancer is a sex hormone dependent or androgen receptor driven cancer, wherein the human subject is further treated with an androgen receptor antagonist, such as enzalutamide, prior to, concurrent, or subsequent to the parenteral administration of the pharmaceutical composition. See e.g., any of the methods described in [72]-[74] and [75]-[92] and [108]-[109](as applicable) of the Summary section. For example, in some embodiments, the androgen receptor antagonist, such as enzalutamide, is administered subsequent to the first parenteral administration of the pharmaceutical composition. In some embodiments, the androgen receptor antagonist, such as enzalutamide, is administered prior to the first parenteral administration of the pharmaceutical composition. As androgen receptor antagonists, such as enzalutamide (Xtandi), are typically administered orally at a different dosing frequency, the dosing regimen for the combined treatment of the human subject with the parenteral administration and such androgen receptor antagonist(s) is not particularly limited. For example, in some embodiments, the androgen receptor antagonist(s) can be administered to the human subject through oral administration (or another route of administration) at a dosing regimen typical to such androgen receptor antagonist(s), such as a U.S. Food and Drug Administration approved dosing regimen for the respective androgen receptor antagonist(s). For example, in some embodiments, the androgen receptor antagonist(s) is enzalutamide, and the enzalutamide can be administered to the human subject orally once a day at a daily dose of about 160 mg. In some embodiments, the enzalutamide treatment can start at least one day prior to the first parenteral administration of the pharmaceutical composition. In some embodiments, the enzalutamide treatment can start on the same day of the first parenteral administration of the pharmaceutical composition. In some embodiments, the enzalutamide treatment can start at least one day after the first parenteral administration of the pharmaceutical composition. In some embodiments, the androgen receptor antagonist(s) can be administered to the human subject in combination with the parenteral administration of the pharmaceutical composition to achieve a synergistic effect on cancer treatment.

Human subjects suitable to be treated with the method herein are not particularly limited, which include those at various stages of diseases or treatments and other characteristics. For example, in some embodiments, the human subject can be a non-castrated human subject. As detailed in PCT/US2022/016278, it was found that administration of abiraterone prodrugs can lead to a sustained reduction of testosterone in subjects within a few days following the first administration of the prodrug without the need for castration or another drug that is effective in lowering testosterone levels. However, in some embodiments, the human subject can also be castrated. For example, in some embodiments, the human subject can be chemically castrated, such as treated with a gonadotropin-releasing hormone agonist and/or antagonist. The term “castration”, “castrate(d)”, and the alike, as used herein should be understood as encompassing all forms of castration, including surgical and chemical castrations. In some embodiments, the method herein can also administer the pharmaceutical composition comprising the abiraterone prodrug to the human subject without regard to whether the human subject is castrated or not. In some embodiments, the human subject has not undergone a prostatectomy. In some embodiments, the human subject can be characterized as suffering from hepatic impairment, such as moderate to severe hepatic impairment (Child-Pugh Class B or C), prior to the administering of the abiraterone prodrug. In some embodiments, the human subject has prostate cancer, and the method herein does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition. In some embodiments, the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition. For example, prostate cancer patients who experience an increase of serum or plasma progesterone level after an abiraterone acetate treatment, such as Zytiga (oral abiraterone acetate and prednisone) treatment, especially those patients with a level of progesterone increased to greater than about 3 nM after 3 months treatment, can be advantageously treated by switching the abiraterone acetate treatment with the methods described herein. In some embodiments, unless contrary from context, the “3 nM” progesterone level herein can refer to the plasma progesterone level. In some embodiments, unless contrary from context, the “3 nM” progesterone level herein can refer to the serum progesterone level. In some embodiments, the human subject can be characterized as being sensitive to or otherwise intolerant with a gonadotropin-releasing hormone antagonist and/or agonist. In some embodiments, the human subject can be characterized as chemotherapy naïve or hormone therapy naïve prior to being administered the pharmaceutical composition herein. However, in some embodiments, the human subject can also be treated with chemotherapy or hormone therapy prior to being administered the pharmaceutical composition herein. For example, in some embodiments, the human subject can have a disease or disorder (e.g., prostate cancer) that has progressed on or after the chemotherapy and/or hormone therapy, such as a taxane-based chemotherapy regimen, for example, docetaxel-based or cabazitaxel-based chemotherapy. In some embodiments, the human subject's disease has progressed on or after an androgen receptor antagonist based treatment, such as enzalutamide based treatment. In some embodiments, the human subject's disease has progressed on or after an oral abiraterone acetate based treatment, such as oral abiraterone acetate and prednisone based treatment. For example, in some embodiments, the human subject has developed resistance to the treatment of abiraterone acetate in combination with prednisone, including resistance due to increased levels of progesterone. As discussed above, the human subject suffering from or being susceptible to one or more side effects associated with inhibition of CYP17A1 hydroxylase activity can also be advantageously treated with the method herein, which selectively inhibit lyase activity over hydroxylase activity. In some embodiments, the methods herein can also include a step of identifying and/or selecting a human subject having any one or more of the characteristics described herein, and administering the pharmaceutical composition herein to the human subject identified and/or selected as having one or more of the characteristics described herein, such as having any of those characteristics described in [4]-[17], [31]-[33], [61]-[62], [64], [65], [69], [79]-[84], and [89] of the Summary section. In some embodiments, the methods herein can also include a step of determining whether a human subject has any one or more of the characteristics described herein, and administering the pharmaceutical composition herein to the human subject determined as having one or more of the characteristics described herein, such as having any of those characteristics described in [4]-[17], [31]-[33], [61]-[62], [64], [65], [69], [79]-[84], and [89] of the Summary section.

In the method herein where the human subject is non-castrated and/or does not wish to become castrated, another drug that is effective in lowering serum and/or gonadal testosterone level, is not administered to the human subject concurrently with the administration of the abiraterone prodrug, during the treatment with the abiraterone prodrug, or otherwise interfering with the treatment with the abiraterone prodrug. For example, in some embodiment, the human subject is not treated with a gonadal testosterone suppressing drug, other than the administered abiraterone prodrug, in an amount effective to reduce serum testosterone level in the human subject. In some embodiment, the human subject is not treated with a gonadotropin-releasing hormone antagonist and/or agonist in an amount effective to reduce serum testosterone level in the human subject. In some embodiment, the human subject is not treated with any gonadal testosterone suppressing drug other than the administered abiraterone prodrug. In some embodiments, the human subject is not treated with any gonadotropin-releasing hormone antagonist and/or agonist. In some embodiments, the human subject is not treated with a drug selected from buserelin, leuprolide, deslorelin, fertirelin, histrelin, gonadorelin, lecirelin, goserelin, nafarelin, peforelin and triptorelin. In some embodiments, the human subject is not treated with a drug selected from abarelix, cetrorelix, degarelix, ganirelix, elagolix, linzagolixa, and relugolix. In some embodiments, the human subject can be sensitive to or otherwise intolerant with a gonadotropin-releasing hormone antagonist and/or agonist.

Various diseases or disorders are suitable to be treated with the method herein. For example, in some embodiments, the disease or disorder can be a sex hormone-dependent benign or malignant disorder, an androgen receptor drive cancer, or a syndrome due to androgen excess. In some embodiments, the hormone-dependent benign or malignant disorders can be androgen-dependent disorders or estrogen-dependent disorders such as androgen or estrogen-dependent cancers. In some embodiments, the sex hormone-dependent benign or malignant disorder can be prostate cancer or breast cancer. In some embodiments, the sex hormone-dependent benign or malignant disorder is CRPC or CSPC. In some embodiments, the sex hormone-dependent benign or malignant disorder can be metastatic CRPC or metastatic CSPC. In some embodiments, the sex hormone-dependent benign or malignant disorder can also be ovarian cancer, bladder cancer, hepatocellular carcinoma, or lung cancer. Various non-oncologic syndromes due to androgen excess can also be treated with the method herein, for example, syndromes due to androgen excess such as endometriosis, polycystic ovary syndrome, classical or nonclassical congenital adrenal hyperplasia, precocious puberty, hirsutism, etc.

In some specific embodiments, the method herein is for treating polycystic ovary syndrome (PCOS), congenital adrenal hyperplasia (CAH), or endometriosis.

In some specific embodiments, the method herein is for treating a sex hormone dependent or androgen receptor driven cancer. In some embodiments, the sex hormone dependent or androgen receptor driven cancer has metastasized to one or more lymph nodes, and the administering of the pharmaceutical composition is effective in inhibiting growth of the cancer in the one or more lymph nodes. In some embodiments, the sex hormone dependent or androgen receptor driven cancer is prostate cancer that has metastasized to one or more lymph nodes, and the administering of the pharmaceutical composition is effective in inhibiting growth of the cancer in the one or more lymph nodes.

In some embodiments, the sex hormone dependent or androgen receptor driven cancer can be androgen receptor positive salivary duct carcinoma, or androgen receptor positive glioblastoma multiforme.

In some specific embodiments, the method herein is for treating prostate cancer, endometrial cancer, or ovarian cancer.

In some embodiments, the method herein is for treating prostate cancer (e.g., any of those described herein).

Prostate cancer suitable to be treated with the method herein is not particularly limited and include without limitation any of those prostate cancer for which abiraterone or its derivatives (particularly abiraterone acetate) has been approved for marketing (e.g., in the U.S. or Europe) or for which abiraterone or its derivatives (e.g., abiraterone acetate) is or has been in a clinical trial, such as those trials registered in the website clinicaltrials.gov as of the filing date of this application. For example, in some embodiments, the prostate cancer can be primary/localized prostate cancer (newly diagnosed or early stage), advanced prostate cancer (e.g., after castration for recurrent prostate cancer, locally advanced prostate cancer, etc.), recurrent prostate cancer (e.g., prostate cancer which was not responsive to a primary therapy), non-metastatic castration-resistant prostate cancer, metastatic prostate cancer, metastatic castration-resistant prostate cancer (CRPC), or hormone-sensitive prostate cancer. In some embodiments, the prostate cancer is a localized prostate cancer, e.g., a high risk localized prostate cancer. In some embodiments, the human subject having prostate cancer is characterized as having a rising amount of prostate specific antigen, e.g., following radical prostatectomy. In some embodiments, the prostate cancer is a metastatic castration-sensitive prostate cancer, non-metastatic castration-sensitive prostate cancer, non-metastatic castration-resistant prostate cancer, or metastatic castration-resistant prostate cancer. In some embodiments, the prostate cancer is a newly diagnosed high risk metastatic hormone sensitive prostate cancer. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. In some embodiments, the prostate cancer is a metastatic CRPC (mCRPC), wherein the human subject is asymptomatic or mildly symptomatic after failure of androgen deprivation therapy in whom chemotherapy is not yet clinically indicated. In some embodiments, the prostate cancer is a metastatic CRPC (mCRPC), wherein the human subject's disease has progressed on or after a taxane-based chemotherapy regimen, such as docetaxel-based or cabazitaxel-based chemotherapy regimen. In some embodiments, the prostate cancer is a refractory prostate cancer. As used herein and unless otherwise specified, the phrase “refractory prostate cancer” means prostate cancer that is not responding to an anti-cancer treatment or prostate cancer that is not responding sufficiently to an anti-cancer treatment. Refractory prostate cancer can also include recurring or relapsing prostate cancer. As used herein and unless otherwise specified, the phrase “relapsing prostate cancer” means prostate cancer that was at one time responsive to an anti-cancer treatment but has become no longer responsive to such treatment or is no longer responding sufficiently to such treatment. As used herein and unless otherwise specified, the phrase “recurring (or recurrent) prostate cancer” means prostate cancer that has returned after a patient has been earlier diagnosed with prostate cancer, undergone treatment or had been previously diagnosed as cancer-free.

Prostate cancer can be characterized with certain Gleason scores. Typically, a pathologist looks at how the cancer cells are arranged in the prostate and assigns a score on a scale of 3 to 5 from 2 different locations, an area where the cancer is most obvious, and another area of growth. Cancer cells that look similar to healthy cells receive a low score. Cancer cells that look less like healthy cells or look more aggressive receive a higher score. The scores from the two areas are added together to an overall score between 6 and 10.

The method herein is not particularly limited to prostate cancers having a particular Gleason score. For example, in some embodiments, the prostate cancer can be characterized as having a Gleason score of ≤6. In some embodiments, the prostate cancer can be characterized as having a Gleason score of ≤7. In some embodiments, the prostate cancer can be characterized as having a Gleason score of ≤8. In some embodiments, the prostate cancer can be characterized as having a Gleason score of ≤9. In some embodiments, the prostate cancer can be characterized as having a Gleason score of ≤10.

Prostate cancer can be characterized with certain levels of prostate specific antigen (PSA). For example, in combination with other indicators, PSA level less than 10 ng/ml, Gleason score K 6, AND clinical stage ≤T2a may be viewed as low risk for having or developing metastatic disease or dying of prostate cancer; PSA level between 10-20 ng/ml, Gleason score 7, OR clinical stage T2b/c may be viewed as intermediate risk; and PSA level greater than 20 ng/ml, Gleason score ≥8, OR clinical stage ≥T3 may be viewed as high risk. In addition, in some cases, a patient with prostate cancer after certain treatment may have undetectable PSA for a period of time. In some cases, a subsequent rise in PSA level, such as ≥0.2 ng/ml, ≥2.0 ng/ml, etc. above nadir, may be indicative of a recurrent prostate cancer. The method herein is not particularly limited to treat human subjects with any particular levels of PSA. In some embodiments, the human subject can be characterized as having an increase in PSA ≥2.0 ng/ml over nadir.

In some embodiments, the method herein is for treating a newly diagnosed high risk metastatic hormone sensitive prostate cancer.

In some embodiments, the method herein can also be used for treating breast cancer. Breast cancer suitable to be treated with the method herein is not particularly limited. For example, in some embodiments, the breast cancer can be molecular apocrine HER2− negative breast cancer, metastatic breast cancer, such as ER+ metastatic breast cancer, ER+ and HER2 negative breast cancer, AR+ triple negative breast cancer, etc.

In some embodiments, a disease or disorder is associated with 21-hydroxylase deficiency can also be treated with the method herein.

In some embodiments, the method herein can be used for treating human subjects having a cancer, such as prostate cancer, breast cancer, adrenal cancer, leukemia, lymphoma, myeloma, Waldenström's macroglobulinemia, monoclonal gammopathy, benign monoclonal gammopathy, heavy chain disease, bone and connective tissue sarcoma, brain tumors, thyroid cancer, pancreatic cancer, pituitary cancer, eye cancer, vaginal cancer, vulvar cancer, cervical cancer, uterine cancer, ovarian cancer, esophageal cancer, stomach cancer, colon cancer, rectal cancer, liver cancer, gallbladder cancer, cholangiocarcinoma, lung cancer, testicular cancer, penal cancer, oral cancer, skin cancer, kidney cancers, Wilms' tumor and/or bladder cancer.

In some embodiments, the method herein can include treating the human subject with one or more additional therapies. For example, in some embodiments, the human subject is further treated a radiation therapy. In some embodiments, the human subject is further treated a surgery. In some embodiments, the method is for treating prostate cancer and includes a combination therapy, which further comprises administering to the human subject one or more additional therapies, e.g., as described herein under the section titled Combination Treatment for Prostate Cancer as described herein below. Non-limiting examples of useful additional therapies also include any of those described in [37]-[49] in the Summary section herein.

In some embodiments according to the method herein, the abiraterone prodrug, in particular, abiraterone decanoate, is administered to the human subject in combination with dexamethasone. For example, in any of the methods described herein, unless specified or otherwise contrary from context, the method can comprise administering abiraterone decanoate and dexamethasone to the human subject. In some embodiments, the method is for treating prostate cancer, such as for treating a human subject who is a chemotherapy naïve CRPC patient, including a chemotherapy naïve mCRPC patient. In some embodiments, the method can comprise administering to the human subject (i) abiraterone decanoate intramuscularly once every one to three months, preferably, about 1260 mg of abiraterone decanoate once every three months; and (ii) dexamethasone orally once daily. In some embodiments, the dexamethasone is orally administered to the human subject at a dose of about 0.1 to 1 mg/day, such as about 0.5 mg/day. Additional therapies can also be used in combination with the abiraterone decanoate and dexamethasone treatment, which without limitation include any of such other therapies described herein, such as any of those applicable therapies described in [37]-[49] in the Summary section herein.

The combination treatment of dexamethasone with the abiraterone prodrug (preferably, abiraterone decanoate) is believed to be advantageous in comparison with a combination with another glucocorticoid, such as prednisone or prednisolone. In the context of oral abiraterone acetate, it was shown that in comparison with prednisone, dexamethasone can be advantageously used to provide better control of upstreat steroids level, better control of ACTH level, better efficacy in treating prostate cancer, with better and more durable PSA responses and progression-free survival (PFS), a lower agonist activity of glucocorticoid receptor (which is a resistance mechanism to abiraterone acetate and prednisone treatment), and a longer half-life, etc. Without wishing to be bound by theories, it is believed that the longer half-life is one reason that dexamethasone is a better glucocorticoid for use in combination with the long acting abiraterone prodrugs herein, in particular abiraterone decanoate. In addition, it was known that switching glucocorticoid partner of abiraterone acetate from prednisone to dexamethasone in patients who have progressed on abiraterone acetate and prednisone treatment can lead to elimination of prostate cancer clinical resistance. See e.g., JAMA Oncology, 5(8), 1159-1167 (2019); British Journal of Cancer, 111(12), 2248-2253 (2014); The Oncologist, 19(12), 1231-1240 (2014); The Oncologist, 20(5), e13-e13 (2015); British Journal of Cancer, 119(9), 1052-1059 (2018); and European Urology, 67(4), 673-679 (2015).

In some embodiments according to the method herein, a glucocorticoid replacement therapy (e.g., administering a glucocorticoid, such as hydrocortisone, prednisone, prednisolone, methylprednisolone, or dexamethasone) is not desired. For example, a glucocorticoid may be contraindicated for the human subject, who may have an underlying condition, such as diabetics. In some embodiments, the method can also be characterized in that the human subject is not treated with a glucocorticoid replacement therapy. In some embodiments, the human subject is not treated with an agent selected from hydrocortisone, prednisone, prednisolone, methylprednisolone, and dexamethasone. In some embodiments, the method herein can comprise administering to the human subject a mineralocorticoid receptor antagonist, such as eplerenone. For example, in any of the embodiments herein when glucocorticoid replacement therapy is not desired and/or not administered, the method can comprise administering to the human subject a mineralocorticoid receptor antagonist, such as eplerenone.

In some embodiments according to the method herein, an agent for treating a mineralocorticoid toxicity is not needed and not administered to the human subject. As discussed herein, in some embodiments, the administering of abiraterone prodrug, such as intramuscularly administering abiraterone decanoate, to human subjects can achieve a lyase selectivity and does not cause a significant increase in mineralocorticoid levels and/or does not cause a toxicity due to mineralocorticoid excess. Thus, in some embodiments, the human subject is not treated with an agent effective for treating a mineralocorticoid toxicity. In particular, in some embodiments, the human subject is not treated with an agent that is a glucocorticoid or a mineralocorticoid receptor antagonist.

Suitable pharmaceutical compositions and abiraterone prodrugs for the method herein is not particularly limited and include any of those described herein, such as any of the abiraterone decanoate formulations described herein, e.g., any of those described in the Summary section, such as [18]-[30] or [93]-[107] of the Summary section herein, any of those described in the Examples section, and any of the abiraterone prodrugs and abiraterone prodrug formulations described in U.S. Pat. No. 10,792,292 B2, and PCT Application Nos. PCT/US2021/048607 and PCT/US2022/016278. Typically, the pharmaceutical composition suitable for the method herein is a long-acting parenteral formulation comprising the abiraterone prodrug. In some embodiments, the long-acting parenteral formulation can be formulated to deliver a therapeutically effective plasma levels of abiraterone over an extended period of time (e.g., at least 1 week, e.g., at least two weeks, at least 3 weeks, at least 4 weeks, and up to six or eight weeks or more, etc.) in the human subject, following a single administration. In some embodiments, the therapeutically effective plasma concentration of abiraterone can be a concentration of at least 1 ng/ml, e.g., at least 2 ng/ml, at least 4 ng/ml, at least 8 ng/ml. In some embodiments, the therapeutically effective blood plasma concentration of abiraterone can also be about 0.5 ng/ml or higher. In some embodiments, the therapeutically effective blood plasma concentration of abiraterone can also be about 0.1 ng/ml or higher. In some embodiments, the pharmaceutical composition can be formulated to be administered to the human subject to provide a PK profile described herein, such as (a) a blood plasma concentration of abiraterone above 0.5 ng/ml for a period of at least two weeks from a single dose; (b) a single dose or steady state Cₘₐₓ of abiraterone between about 1 ng/ml and about 300 ng/ml, such as between about 1 ng/ml and about 10 ng/ml; or (c) both (a) and (b).

Routes of administration and dosing regimen for the method herein are also not particularly limited and include any of those described herein. Typically, the pharmaceutical composition can be administered to the human subject through an intramuscular injection, intradermal injection, or subcutaneous injection. For example, in some specific embodiments, the pharmaceutical composition is administered to the human subject through an intramuscular injection. The parenteral administration herein can in some embodiments be advantageous. For example, in some embodiments, the parenteral administering can be carried out without regard to whether the human subject has food, thus, in some embodiments, the abiraterone prodrugs or abiraterone prodrug formulations of the present disclosure can be administered to the human subject with or without food. In other words, the fed or fasted status of the human subject is not important. This removes the restriction associated with the currently marketed Zytiga® formulation, which states that the medication “must be taken on an empty stomach with water at least 1 hour before or 2 hours after a meal.” Therefore, among other advantages, the method herein can improve patient compliance.

Dosing amounts and frequencies for the method herein are also not particularly limited and include any of those described herein. Generally, the pharmaceutical composition is administered to the human subject once a week or once in more than a week. For example, in some embodiments, the method herein is comprise administering the abiraterone prodrug or abiraterone prodrug formulation herein at a dosing frequency ranging from once a week to once every few months. In some embodiments, the pharmaceutical composition is administered to the human subject once a month or once in more than a month, such as once every two months or once every three months.

In some particular embodiments, the method herein can comprise administering the abiraterone prodrug or abiraterone prodrug formulation herein in a dosing frequency ranging from once a month to once every few months, such as once every month, once every two months, once every three months, or even less frequent dosing. The dosing amounts of the abiraterone prodrugs herein (e.g., abiraterone decanoate) for each administration can vary, typically ranging from 0.5 mg/kg to 200 mg/kg, such as about 0.5 mg/kg to about 200 mg/kg (e.g., about 0.5 mg/kg, about 1 mg/kg, about 5 mg/kg, about 10 mg/kg, about 20 mg/kg, about 30 mg/kg, about 50 mg/kg, about 90 mg/kg, about 100 mg/kg, about 200 mg/kg, or any ranges between the recited values) of body weight of a human subject. In some embodiments, the administering can provide any of the pharmacokinetic profile described herein, for example, (a) a blood plasma concentration of abiraterone above 0.5 ng/ml for a period of at least two weeks (e.g., up to 10 weeks or beyond) from a single dose; (b) a single dose or steady state Cₘₐₓ of abiraterone between about 1 ng/ml and about 300 ng/ml, such as between about 1 ng/ml and about 10 ng/ml; or (c) both (a) and (b). In some embodiments, the administering can also provide a concentration of abiraterone in a tissue of the human subject at least 10 times higher than the blood plasma concentration of abiraterone at 7 days post administration (i.e., at 168 hours from the time of administration), wherein the tissue is selected from liver, lung, testes, inguinal lymph, iliac lymph, adrenal, and prostate.

Typically, the amount of abiraterone prodrug (e.g., abiraterone decanoate) administered can be adjusted to be effective in achieving a sustained reduction of serum testosterone level in the human subject to 50% below baseline or lower, such as about 60% below baseline or lower, about 70% below baseline or lower, about 80% below baseline or lower, or about 90% below baseline or lower, about 95% below baseline or lower, within 15 days of the first administration of the pharmaceutical composition. In some embodiments, the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition. In some embodiments, the abiraterone prodrug (e.g., abiraterone decanoate) is administered in an effective amount to reduce the serum testosterone level in the human subject to 80% below baseline or lower, when measured at 24 weeks after the first administration of the abiraterone prodrug. In some embodiments, the abiraterone prodrug (e.g., abiraterone decanoate) is administered in an effective amount to reduce the serum testosterone level in the human subject to 85% below baseline or lower, when measured at 24 weeks after the first administration of the abiraterone prodrug. In some embodiments, the abiraterone prodrug (e.g., abiraterone decanoate) is administered in an effective amount to reduce the serum testosterone level in the human subject to 90% below baseline or lower, when measured at 24 weeks after the first administration of the abiraterone prodrug. In some embodiments, the abiraterone prodrug (e.g., abiraterone decanoate) is administered in an effective amount to reduce the serum testosterone level to about 50 ng/dL or below (e.g., about 40 ng/dL or below, about 30 ng/dL or below, about 20 ng/dL or below, about 10 ng/dL or below, etc.), when the human subject is a non-castrated human subject or about 1 ng/dL or below, when the human subject is a castrated human subject, when measured at 24 weeks after the first administration of the abiraterone prodrug. As exemplified herein, the abiraterone prodrug (e.g., abiraterone decanoate) can be administered to the human subject without significantly raising the level of mineralcorticoid and any such increase returns to baseline level within 4-6 weeks following the first administration of the abiraterone prodrug. In some preferred embodiments, the administering of the abiraterone prodrug (e.g., abiraterone decanoate) does not enhance serum progesterone level in the human subject by more than 40% above baseline at 4 weeks following the first administration of the abiraterone prodrug. In some embodiments, the administering of the abiraterone prodrug (e.g., abiraterone decanoate) does not enhance serum progesterone level in the human subject by more than 40% above baseline from 2 weeks to 12 weeks following the second administration of the abiraterone prodrug. In some embodiments, the administering of the pharmaceutical composition does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the abiraterone prodrug.

In some embodiments, in particular in the methods of treating prostate cancer herein, the dosing amount and frequency of the abiraterone prodrugs herein (e.g., abiraterone decanoate) can be adjusted such that the administering provides an effective amount of abiraterone to reduce the serum testosterone level to about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject (e.g., a human subject chemically castrated with a GnRH agonist and/or antagonist), within 15 days (e.g., within 7 days, between 7-15 days, etc.) of the first administration of the abiraterone prodrug. For example, in some embodiments, the administering provides an effective amount of abiraterone to reduce the serum testosterone level to about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, when measured on day 15 after the first administration of the abiraterone prodrug. In some embodiments, the administering provides an effective amount of abiraterone to achieve a sustained reduction of serum testosterone level, such as achieving and maintaining the serum testosterone level at about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, within 15 days (e.g., within 7 days, between 7-15 days, etc.) of the first administration of the abiraterone prodrug. In some embodiments, the dosing amount and frequency of the abiraterone prodrugs herein (e.g., abiraterone decanoate) can be adjusted such that the administering provides an effective amount of abiraterone to reduce 50% or more, preferably, 75% or more of serum testosterone level from baseline within 15 days (e.g., within 7 days, between 7-15 days, etc.) of the first administration of the abiraterone prodrug. For example, in some embodiments, the administering provides an effective amount of abiraterone to reduce 50% or more, preferably, 75% or more of serum testosterone level from baseline when measured on day 15 after the first administration of the abiraterone prodrug. In some embodiments, the administering provides an effective amount of abiraterone to achieve a sustained reduction of serum testosterone level, such as by 50% or more, 75% or more, from baseline within 15 days (e.g., within 7 days, between 7-15 days, etc.) of the first administration of the abiraterone prodrug. To be clear, the phrase “sustained reduction of serum testosterone level” should be understood as referring to that the serum testosterone levels remain at a reduced level, such as at about 50 ng/dL or below in a non-castrated human subject or about 1 ng/dL or below in a castrated human subject, or 50% or less compared to baseline, for a sustained period of time, which can be 1 day or longer, 3 days or longer, 7 days or longer, and up to a month, or several months. As exemplified herein, the serum testosterone levels can be reduced in a dose-dependent fashion for a prolonged period of time.

In some embodiments, the method herein can be characterized in that the administering of the pharmaceutical composition reduces the level of prostate specific antigen in the human subject. For example, in some preferred embodiments, the level of prostate specific antigen can be reduced to 50% or below, preferably, 90% or below, compared to the baseline PSA level, at least at one time point following the first administration of the pharmaceutical composition and/or following one or more subsequent administration of the pharmaceutical composition. In some preferred embodiments, the level of prostate specific antigen can be reduced to below the lower limit of quantitation (LLOQ) at least at one time point during the treatment period.

In some embodiments, the method herein can be characterized as achieving any one or more of the following outcomes: (a) Objective response per RECIST v1.1 with a minimum interval for confirmation of CR and PR of 4 weeks; (b) PSA decline of ≥50% from baseline, confirmed by a second consecutive PSA assessment at least 3 weeks later; (c) Conversion of circulating tumor cell count (CTC) to <5 cells/7.5 mL blood nadir confirmed by an additional assessment at least 3 weeks later (for subjects with a CTC count of ≥5 cells/7.5 mL blood at baseline); and (4) Radiographic progression-free survival (rPFS). In some embodiments, the method herein can be characterized as achieving any one or more of the following outcomes better than the corresponding treatments with oral Zytiga® tablets: (1) Overall survival, defined as the time from the first dose of study drug to the date of death due to any cause; (2) Best Overall Response (BOR) per RECIST v1.1, defined as the best radiographic response across all time-point responses; and (3) DOR (defined as the length of time from date of first documented, confirmed response) using CTC and/or PSA and/or RECIST v1.1 and PCWG3 until date of documented progression or death from any cause.

In any of the embodiments herein, unless specified or obviously contrary from context, the method herein can comprise administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values. In some preferred embodiments, the method herein can comprise administering abiraterone decanoate to the human subject intramuscularly, at a dosing frequency of once every three months, wherein each administration comprises administering to the human subject about 1260 mg of abiraterone decanoate. In some preferred embodiments, the method herein can comprise administering abiraterone decanoate to the human subject intramuscularly, at a dosing frequency of once every three months, wherein each administration comprises administering to the human subject about 1260 mg of abiraterone decanoate, and orally administering dexamethasone to the human subject at a daily dose of about 0.5 mg/day.

### Methods of Reducing Steroid Hormone Levels

Some embodiments of the present disclosure are directed to methods of reducing serum steroid hormone level in a human subject in need thereof, in particular, for reducing serum steroid hormone level by selectively inhibiting CYP17A1 lyase activity over hydroxylase activity.

In some embodiments, the present disclosure provides a method of reducing serum testosterone level in a human subject in need thereof, the method comprising parenterally administering to the human subject an effective amount of a pharmaceutical composition comprising an abiraterone prodrug (e.g., an abiraterone lipophilic ester), wherein the pharmaceutical composition is administered in an effective amount to achieve a sustained reduction of serum testosterone level in the human subject to 50% below baseline or lower within 15 days of the first administration of the pharmaceutical composition, wherein the administering of the pharmaceutical composition does not enhance serum progesterone level in the human subject (i) by more than 40% above baseline following the first administration of the pharmaceutical composition; and/or (ii) by more than 40% above baseline from 2 weeks to 12 weeks following the second administration of the pharmaceutical composition, preferably, does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the pharmaceutical composition. Typically, the amount of abiraterone prodrug (e.g., abiraterone decanoate) administered can be adjusted to be effective in achieving a sustained reduction of serum testosterone level in the human subject to 50% below baseline or lower, such as about 60% below baseline or lower, about 70% below baseline or lower, about 80% below baseline or lower, or about 90% below baseline or lower, about 95% below baseline or lower, within 15 days of the first administration of the pharmaceutical composition. In some embodiments, the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition. In some embodiments, the abiraterone prodrug (e.g., abiraterone decanoate) is administered in an effective amount to reduce the serum testosterone level in the human subject to 80% below baseline or lower, such as 85% below baseline or lower, or 90% below baseline or lower, when measured at 24 weeks after the first administration of the abiraterone prodrug. In some embodiments, the abiraterone prodrug (e.g., abiraterone decanoate) is administered in an effective amount to reduce the serum testosterone level to about 50 ng/dL or below (e.g., about 40 ng/dL or below, about 30 ng/dL or below, about 20 ng/dL or below, about 10 ng/dL or below, etc.), when the human subject is a non-castrated human subject, or about 1 ng/dL or below, when the human subject is a castrated human subject, when measured at 24 weeks after the first administration of the abiraterone prodrug.

In some particular embodiments, the present disclosure provides a method of reducing serum testosterone level in a human subject in need thereof, the method comprising parenterally administering to the human subject a pharmaceutical composition comprising an abiraterone prodrug (e.g., an abiraterone lipophilic ester), wherein the administering of the pharmaceutical composition does not enhance serum progesterone level in the human subject (i) by more than 40% above baseline following the first administration of the pharmaceutical composition; and/or (ii) by more than 40% above baseline from 2 weeks to 12 weeks following the second administration of the pharmaceutical composition, preferably, does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the pharmaceutical composition. Typically, in particular when the human subject is characterized as having prostate cancer, the administering provides an effective amount of abiraterone to reduce the serum testosterone level to about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, within 15 days (e.g., within 7 days, between 7-15 days, etc.) of the first administration of the abiraterone prodrug. For example, in some embodiments, the administering provides an effective amount of abiraterone to reduce the serum testosterone level to about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, when measured on day 15 after the first administration of the abiraterone prodrug. In some embodiments, the administering provides an effective amount of abiraterone to achieve a sustained reduction of serum testosterone level, such as achieving and maintaining the serum testosterone level at about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, within 15 days (e.g., within 7 days, between 7-15 days, etc.) of the first administration of the abiraterone prodrug. Typically, the administering provides an effective amount of abiraterone to reduce 50% or more, preferably, 75% or more of serum testosterone level from baseline within 15 days (e.g., within 7 days, between 7-15 days, etc.) of the first administration of the abiraterone prodrug. For example, in some embodiments, the administering provides an effective amount of abiraterone to reduce 50% or more, preferably, 75% or more of serum testosterone level from baseline when measured on day 15 after the first administration of the abiraterone prodrug. In some embodiments, the administering provides an effective amount of abiraterone to achieve a sustained reduction of serum testosterone level, such as by 50% or more, 75% or more, from baseline within 15 days (e.g., within 7 days, between 7-15 days, etc.) of the first administration of the abiraterone prodrug. In some embodiments, the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition. In some embodiments, the sustained reduction of serum testosterone level is characterized in that once the serum testosterone level in the human subject about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, the serum testosterone level remains at about 50 ng/dL or below in a non-castrated human subject or about 1 ng/dL or below in a castrated human subject up to 8 weeks or longer following the first administration of the pharmaceutical composition.

Human subjects suitable to be treated with the method herein for reducing serum testosterone levels are not particularly limited and include any of those described herein. In some embodiments, the human subject can be a non-castrated human subject. In some embodiments, the human subject can also be castrated. For example, in some embodiments, the human subject can be chemically castrated, such as treated with a gonadotropin-releasing hormone agonist and/or antagonist. In some embodiments, the human subject can be characterized as suffering from hepatic impairment, such as moderate to severe hepatic impairment (Child-Pugh Class B or C), prior to the administering of the abiraterone prodrug. In some embodiments, the human subject has prostate cancer, and the method herein does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition. In some embodiments, the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition herein. In some embodiments, the human subject can be characterized as being sensitive to or otherwise intolerant with a gonadotropin-releasing hormone antagonist and/or agonist. In some embodiments, the human subject can be characterized as chemotherapy naïve or hormone therapy naïve prior to being administered the pharmaceutical composition herein. However, in some embodiments, the human subject can also be treated with chemotherapy or hormone therapy prior to being administered the pharmaceutical composition herein. For example, in some embodiments, the human subject can have a disease or disorder (e.g., prostate cancer) that has progressed on or after the chemotherapy and/or hormone therapy, such as a taxane-based chemotherapy regimen, for example, docetaxel-based or cabazitaxel-based chemotherapy. In some embodiments, the human subject's disease has progressed on or after an androgen receptor antagonist based treatment, such as enzalutamide based treatment. In some embodiments, the human subject's disease has progressed on or after an oral abiraterone acetate based treatment, such as oral abiraterone acetate and prednisone based treatment. For example, in some embodiments, the human subject has developed resistance to the treatment of abiraterone acetate in combination with prednisone, including resistance due to increased levels of progesterone. As discussed above, the human subject suffering from or being susceptible to one or more side effects associated with inhibition of CYP17A1 hydroxylase activity can also be advantageously treated with the method herein, which selectively inhibit lyase activity over hydroxylase activity.

The human subject in need of reduction of testosterone typically suffers from one or more diseases or disorders mediated or associated with androgens. For example, in some embodiments, the human subject suffers from a disease or disorder that is sex hormone-dependent or androgen receptor driven, such as a sex hormone-dependent benign or malignant disorder or a syndrome due to androgen excess. Suitable diseases or disorders include any of those described herein, for example, any of those described in [4]-[17] of the Summary Section. In some embodiments, the human subject is characterized as having a sex hormone dependent cancer or androgen receptor driven cancer, e.g., any of those described herein. In some embodiments, the sex hormone dependent or androgen receptor driven cancer has metastasized to one or more lymph nodes. In some embodiments, the administering of the pharmaceutical composition is effective in inhibiting growth of the cancer in the one or more lymph nodes. In some embodiments, the human subject is characterized as having androgen receptor positive salivary duct carcinoma, or androgen receptor positive glioblastoma multiforme. In some embodiments, the human subject is characterized as having prostate cancer (e.g., any of those described herein). For example, in some embodiments, the prostate cancer can be primary/localized prostate cancer (newly diagnosed or early stage), advanced prostate cancer (e.g., after castration for recurrent prostate cancer, locally advanced prostate cancer, etc.), recurrent prostate cancer (e.g., prostate cancer which was not responsive to a primary therapy), non-metastatic castration-resistant prostate cancer, metastatic prostate cancer, metastatic castration-resistant prostate cancer (CRPC), or hormone-sensitive prostate cancer. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. In some embodiments, the human subject suffers from endometrial cancer or ovarian cancer. In some embodiments, the human subject suffers from polycystic ovary syndrome (PCOS), congenital adrenal hyperplasia (CAH), or endometriosis. In some embodiments, the human subject has a newly diagnosed high risk metastatic hormone sensitive prostate cancer. In some embodiments, the human subject has not undergone a prostatectomy. In some embodiments, the human subject is further treated with a radiation therapy.

Suitable pharmaceutical compositions and abiraterone prodrugs for the method herein for reducing serum testosterone level are not particularly limited and include any of those described herein, such as any of the abiraterone decanoate formulations described herein, e.g., any of those described in the Summary section, such as [18]-[30] or [93]-[107] of the Summary section herein, any of those described in the Examples section, and any of the abiraterone prodrugs and abiraterone prodrug formulations described in U.S. Pat. No. 10,792,292 B2, and PCT Application Nos. PCT/US2021/048607 and PCT/US2022/016278. Typically, the pharmaceutical composition is a long-acting parenteral formulation comprising the abiraterone prodrug. In some embodiments, the long-acting parenteral formulation can be formulated to deliver effective plasma levels of abiraterone over an extended period of time (e.g., at least 1 week, e.g., at least two weeks, at least 3 weeks, at least 4 weeks, and up to six or eight weeks or more, etc.) to reduce serum testosterone levels (e.g., to about 50 ng/dL or below in a non-castrated human subject or about 1 ng/dL or below in a castrated human subject, or by 50% or more compared to baseline) in the human subject, following a single administration. In some embodiments, the effective plasma concentration of abiraterone can be a concentration of at least 1 ng/ml, e.g., at least 2 ng/ml, at least 4 ng/ml, at least 8 ng/ml. In some embodiments, the effective blood plasma concentration of abiraterone can also be about 0.5 ng/ml or higher. In some embodiments, the effective blood plasma concentration of abiraterone can also be about 0.1 ng/ml or higher. In some embodiments, the pharmaceutical composition can be formulated to be administered to the human subject to provide (a) a blood plasma concentration of abiraterone above 0.5 ng/ml for a period of at least two weeks from a single dose; (b) a single dose or steady state Cₘₐₓ of abiraterone between about 1 ng/ml and about 300 ng/ml; such as between about 1 ng/ml and about 10 ng/ml; or (c) both (a) and (b).

Routes of administration and dosing regimen for the method herein for reducing serum testosterone levels are also not particularly limited and include any of those described herein. Typically, the pharmaceutical composition can be administered to the human subject through an intramuscular injection, intradermal injection, or subcutaneous injection. For example, in some specific embodiments, the pharmaceutical composition is administered to the human subject through an intramuscular injection. Generally, the pharmaceutical composition is administered to the human subject once a week or once in more than a week. For example, in some embodiments, the pharmaceutical composition is administered to the human subject once a month or once in more than a month, such as once every two months or once every three months.

In some embodiments, the present disclosure provides a method of reducing the level of androgens (e.g., testosterone and/or dihydrotestosterone) and/or estrogens in a human subject in need thereof, the method comprising administering to the human subject any of the abiraterone prodrugs or abiraterone prodrug formulations of the present disclosure. In some embodiments, the human subject suffers from an androgen receptor driven cancer. In some embodiments, the human subject suffers from a syndrome due to androgen excess, such as congenital adrenal hyperplasia (e.g., classical or nonclassical congenital adrenal hyperplasia), endometriosis, polycystic ovary syndrome precocious puberty, hirsutism, etc. In some embodiments, the human subject suffers from an androgen and/or estrogen associated cancer, such as prostate cancer or breast cancer. In some embodiments, the human subject suffers from a sex hormone dependent cancer described herein. Suitable pharmaceutical compositions, subjects, dosing regimen, and routes of administrations for the method include any of those described herein in any combination, such as any of those described in connection with the methods shown in the Summary section herein.

### Abiraterone Prodrugs and Formulations

Various abiraterone prodrugs and abiraterone prodrug formulations are also provided herein, which can be used for the method herein. For example, in some embodiments, the abiraterone drug can be an abiraterone lipophilic ester, such as an acetate, a propionate, a butanoate, a (vaterate) pentanoate, an isocaproate, a buciclate, a cyclohexanecarboxylate, a phenyl propionate, caproate (hexanoate), an enanthate (heptanoate), a cypionate, an octanoate, a nonanoate, a decanoate, an undecanoate, a dodecanoate, a tridecanoate, a tetradecanoate, a pentadecanoates, or a hexadecanoate of abiraterone. Other suitable abiraterone prodrugs include any of those described in U.S. Pat. No. 10,792,292 B2, and PCT Application Nos. PCT/US2021/048607 and PCT/US2022/016278, the content of each of which is herein incorporated by reference in its entirety. Any of the abiraterone decanoate formulations described in U.S. Provisional Application Nos. 63,425,839 can be used for the methods herein.

In any of the embodiments described herein, unless specified or otherwise contrary from context, the abiraterone prodrug can be abiraterone decanoate, or a pharmaceutically acceptable salt thereof,

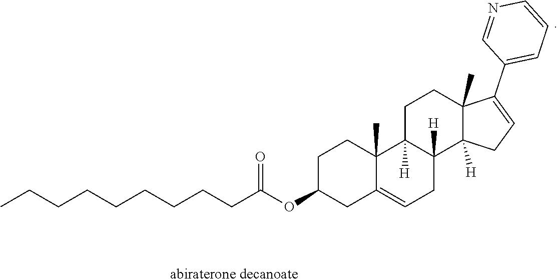

Typically, the pharmaceutical composition (or alternatively referred to herein as abiraterone prodrug formulation) comprising the abiraterone prodrug is formulated for parenteral administration. For example, in some embodiments, the pharmaceutical composition can be formulated for intramuscular injection, intradermal injection, or subcutaneous injection.

In some embodiments, the pharmaceutical composition comprises an abiraterone decanoate solution, which includes abiraterone decanoate dissolved in a pharmaceutically acceptable carrier and a pharmaceutically acceptable antioxidant. For the purposes herein, a liquid mixture of abiraterone decanoate, a pharmaceutically acceptable carrier, a pharmaceutically acceptable antioxidant, and optionally other ingredients, can be characterized as an abiraterone decanoate solution if the amount of abiraterone decanoate in the liquid mixture is below its saturation point (i.e., the maximum solubility of abiraterone decanoate) in the pharmaceutically acceptable carrier at 25° C., taking into considerations of any effect on the solubility of abiraterone decanoate in the pharmaceutically acceptable carrier caused by the antioxidant and optional other ingredients.

The pharmaceutical composition is generally a non-aqueous formulation, for example, an oil-based formulation, and include a non-aqueous pharmaceutically acceptable carrier (e.g., described herein). The pharmaceutical composition typically comprises the abiraterone prodrug and a pharmaceutically acceptable carrier. The pharmaceutically acceptable carrier is not particularly limited. For example, in some embodiments, the pharmaceutically acceptable carrier comprises a pharmaceutically acceptable oil, such as a pharmaceutically acceptable oil for injection, including oils of vegetable origin or synthetic mono- or diglycerides of fatty acids. In some embodiments, the pharmaceutically acceptable oil can be nature oil, synthetic oil, or semi-synthetic oil, such as fractionated coconut oil and medium-chain triglycerides, such as those sold under the trademark Miglyol. In some embodiments, the pharmaceutically acceptable carrier comprises a triglyceride derived from fatty acids. In some embodiments, the pharmaceutically acceptable carrier comprises a triglyceride derived from long and/or medium chain fatty acids, which can be independently poly-unsaturated, mono-unsaturated, or saturated. As understood by those skilled in the art, medium chain fatty acids typically include 6-12 carbons, such as caproic acid, caprylic acid, capric acid, lauric acid, etc.; short chain fatty acids typically have fewer than 6 carbons, whereas long-chain fatty acids typically include 13-21 carbons. In some embodiments, the pharmaceutically acceptable carrier comprises a pharmaceutically acceptable oil, which can be selected from vegetable oil, castor oil, corn oil, sesame oil, cottonseed oil, peanut oil (arachis oil), poppy seed oil, tea seed oil, and soybean oil. In some specific embodiments, the pharmaceutically acceptable carrier can comprise corn oil, which includes a triglyceride, in which the fatty acid constituents are primarily linoleic acid, oleic acid, palmitic acid, and stearic acid.

In some embodiments, in addition to the pharmaceutically acceptable oil, the pharmaceutically acceptable carrier can further comprise a pharmaceutically acceptable solvent (or co-solvent if the oil is counted as a solvent), such as an alcohol, ester, acid, etc. In some embodiments, the pharmaceutically acceptable solvent can include benzyl alcohol, benzyl benzoate, ethanol, glycerol, polyethylene glycol, polysorbate 80, acetic acid, and/or ethyl acetate. In some embodiments, the pharmaceutically acceptable solvent can be benzyl alcohol and/or benzyl benzoate. In some embodiments, the pharmaceutically acceptable solvent can be benzyl alcohol. In some embodiments, the pharmaceutically acceptable solvent can be a combination of benzyl alcohol and benzyl benzoate. The solubility of abiraterone prodrugs such as abiraterone decanoate in a pharmaceutically acceptable oil can be significantly enhanced by a combination of benzyl alcohol and benzyl benzoate.

In some embodiments, the pharmaceutically acceptable carrier can comprise the pharmaceutically acceptable oil and the further pharmaceutically acceptable solvent, wherein the pharmaceutically acceptable oil is selected from vegetable oil, castor oil, corn oil, sesame oil, cottonseed oil, peanut oil, poppy seed oil, tea seed oil, and soybean oil, and the further pharmaceutically acceptable solvent comprises benzyl alcohol, benzyl benzoate, or a combination thereof. In some embodiments, the pharmaceutically acceptable carrier comprises corn oil, benzyl alcohol, and benzyl benzoate. In some embodiments, the benzyl alcohol is present in an amount of about 5-10% by volume, the benzyl benzoate is present in an amount of about 10-20% by volume, and corn oil is present in an amount of about 70-85% by volume, with the combined volume of benzyl alcohol, benzyl benzoate, and corn oil being 100%.

Pharmaceutically acceptable antioxidants suitable for the pharmaceutical compositions herein are not particularly limited. For example, in some embodiments, the pharmaceutical compositions herein can include a thio-containing antioxidant, such as 3-mercapto-1,2-propanediol, alternatively named monothioglycerol, which has a chemical structure of

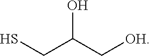

In some embodiments, the pharmaceutical compositions herein can include a tocopherol type antioxidant, such as alpha-tocopherol.

### Exemplary Formulations Comprising Abiraterone Decanoate

In some specific embodiments, the pharmaceutical composition comprises abiraterone decanoate having the formula of:

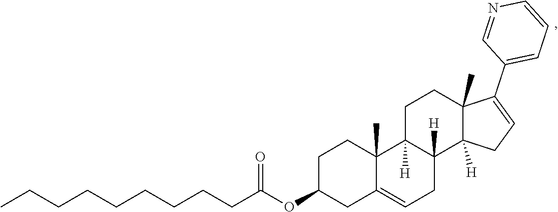

or a pharmaceutically acceptable salt thereof, and a pharmaceutically acceptable carrier. The abiraterone decanoate is typically present in the pharmaceutical composition in its basic form and should be understood as such unless otherwise obvious to the contrary from context. In some embodiments, the abiraterone decanoate can also be in a substantially pure form described herein. For example, the pharmaceutical composition can be prepared from mixing the substantially pure abiraterone decanoate with the pharmaceutically acceptable carrier and optional other ingredients. In some specific embodiments, the substantially pure abiraterone decanoate is in a crystalline form described herein, preferably, crystalline Form A, and the pharmaceutical composition can be prepared from mixing (e.g., dissolving, suspending, or otherwise forming a mixture) the crystalline form (e.g., Form A) with the pharmaceutically acceptable carrier and optional other ingredients.

In some specific embodiments, the pharmaceutical composition comprises (1) abiraterone decanoate; (2) a pharmaceutically acceptable oil (e.g., described herein); (3) a pharmaceutically acceptable solvent (e.g., described herein); and (4) 3-mercapto-1,2-propanediol.

In some specific embodiments, the pharmaceutical composition comprises (1) abiraterone decanoate: (2) benzyl alcohol; (3) benzyl benzoate; (4) corn oil; and (5) 3-mercapto-1,2-propanediol.

Abiraterone decanoate is typically prepared in a high purity form, e.g., suitable for pharmaceutical use. In some embodiments, the present disclosure provides abiraterone decanoate in a substantially pure form, such as having a purity of greater than 80%, preferably greater than 90% (e.g., greater than 95%, greater than 97%, greater than 98%, greater than 99%, greater than 99.5%), by weight, by HPLC area, or both. In some embodiments, the abiraterone decanoate can be characterized by a purity by weight and/or by HPLC area of about 95%, about 97%, about 99%, about 99.5%, about 99.9%, or any ranges between the specified values. For example, in some embodiments, the abiraterone decanoate can be characterized by a purity by weight of about 95%, about 97%, about 99%, about 99.5%, about 99.9%, or any ranges between the specified values. In some embodiments, the abiraterone decanoate can also be characterized as having a low palladium content, such as less than 150 ppm, less than 100 ppm, less than 50 ppm, or less than 10 ppm. In some embodiments, the abiraterone decanoate conforms to the specification shown in Table D herein (see Example 1B). Exemplary procedures for preparing the substantially pure abiraterone decanoate are shown in the Examples section. HPLC methods suitable for measuring the purity of the abiraterone decanoate are also described in the Examples section. The substantially pure abiraterone decanoate can be in a solid form (e.g., a crystalline form described herein, preferably, Form A, amorphous form, or a combination thereof) or in a solution, suspension, or another form. For the avoidance of doubt, an abiraterone prodrug formulation comprising the substantially pure abiraterone decanoate herein and one or more other ingredients should be understood as a mixture of the substantially pure abiraterone decanoate herein and the one or more other ingredients, for example, such formulation can be obtained directly or indirectly from mixing (e.g., dissolving, suspending, or otherwise forming a mixture) the substantially pure abiraterone decanoate with the one or more other ingredients, such as pharmaceutically acceptable oil, solvent, etc.

In some specific embodiments, the pharmaceutical composition comprises abiraterone decanoate, a pharmaceutically acceptable oil (e.g., described herein), benzyl alcohol, and benzyl benzoate. In some embodiments, the pharmaceutically acceptable oil is corn oil. In some embodiments, the benzyl alcohol is present in an amount of about 5-10% by volume, the benzyl benzoate is present in an amount of about 10-20% by volume, and corn oil is present in an amount of about 70-85% by volume, with the combined volume of benzyl alcohol, benzyl benzoate, and corn oil being 100%.

The pharmaceutical composition typically includes abiraterone decanoate at a concentration of about 25 mg/ml to about 500 mg/ml. In some embodiments, the abiraterone decanoate can be present in a concentration of about 50 mg/ml, about 100 mg/ml, about 150 mg/ml, about 200 mg/ml, about 250 mg/ml, about 300 mg/ml, about 350 mg/ml, about 400 mg/ml, about 500 mg/ml, or any ranges between the recited values. In some embodiments, the abiraterone decanoate can be present in a concentration of about 100 mg/ml to about 300 mg/ml, such as about 150 mg/ml to about 250 mg/ml, about 200 mg/ml to about 300 mg/ml, etc.

In some embodiments, the pharmaceutical composition can comprise abiraterone decanoate in its basic form, corn oil, benzyl alcohol, and benzyl benzoate. In some embodiments, the abiraterone decanoate is present in the pharmaceutical composition in an amount of about 50-300 mg/mL. In some embodiments, the benzyl alcohol is in an amount of about 50-150 mg/mL. In some embodiments, the benzyl benzoate is in an amount of about 100-300 mg/mL. In some embodiments, the pharmaceutical composition can comprise, for each milliliter, (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); and (d) corn oil, q.s. to 1 milliliter. In some embodiments, the weight ratio of benzyl alcohol to benzyl benzoate in the pharmaceutical composition ranges from about 2:1 to about 1:5 (e.g., about 1:1 to 1:3, such as about 1:2).

In some embodiments, the pharmaceutical composition comprises an abiraterone decanoate solution, wherein each milliliter of the abiraterone decanoate solution comprises, consists essentially of, or consists of: (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 20 mg (e.g., about 0.5 mg, about 1 mg, about 2 mg, or about 5 mg); and (c) corn oil, q.s. to 1 milliliter, wherein the abiraterone decanoate has the structure of:

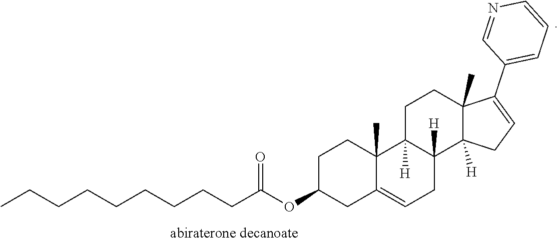

In some embodiments, each milliliter of the abiraterone decanoate solution herein comprises, consists essentially of, or consists of: (a) abiraterone decanoate in its basic form, in an amount of about 180 mg; (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 2 mg (e.g., about 0.5 mg, about 1 mg, or about 2 mg); and (e) corn oil, q.s. to 1 milliliter.

The weight ratio of benzyl alcohol to benzyl benzoate in the abiraterone decanoate solution typically ranges from about 2:1 to about 1:5 (e.g., about 1:1 to 1:3, such as about 1:2).

In some specific embodiments, each milliliter of the abiraterone decanoate solution herein comprises, consists essentially of, or consists of: (a) abiraterone decanoate in its basic form, in an amount of about 180 mg; (b) benzyl alcohol in an amount of about 100 mg; (c) benzyl benzoate in an amount of about 200 mg; (d) 3-mercapto-1,2-propanediol in an amount of about 0.5 mg to about 2 mg (e.g., about 0.5 mg, about 1 mg, or about 2 mg); and (e) corn oil, q.s. to 1 milliliter.

In some embodiments, the pharmaceutical composition can be prepared by mixing (e.g., dissolving) the abiraterone decanoate, such as substantially pure abiraterone decanoate, with the pharmaceutically acceptable carrier and the pharmaceutically acceptable antioxidant. For example, in some embodiments, the pharmaceutical composition can be prepared by mixing (e.g., dissolving) the substantially pure abiraterone decanoate in its basic form with corn oil, benzyl alcohol, benzyl benzoate, and 3-mercapto-1,2-propanediol, wherein the amount of each ingredient can be any of those described herein.

The abiraterone decanoate solution can be typically included in a container having a headspace, e.g., a vial, an ampule, a bottle, etc. In preferred embodiments, the headspace of the container is filled with an inert gas, such as nitrogen gas.

In some embodiments, the abiraterone decanoate solution can also be included in a syringe.

### Exemplary Formulations Comprising Substantially Pure Abiraterone Decanoate

In some specific embodiments, the pharmaceutical composition comprises a substantially pure abiraterone decanoate, which has the following formula:

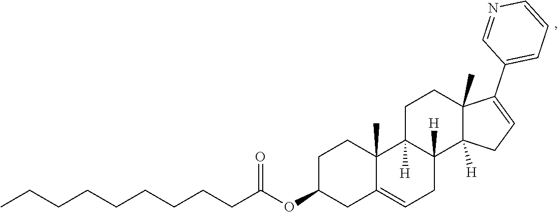

or a pharmaceutically acceptable salt thereof, which is dispersed or dissolved in a pharmaceutically acceptable carrier. In some embodiments, the pharmaceutical composition comprises the substantially pure abiraterone decanoate in its basic form, which is dispersed or dissolved in the pharmaceutically acceptable carrier. In some embodiments, the substantially pure abiraterone decanoate has a purity by weight of at least 95%, preferably, at least 98%, such as about 98.5%, about 99%, about 99.5%, or higher. In some embodiments, the substantially pure abiraterone decanoate can be characterized by a purity by weight and/or by HPLC area of about 95%, about 97%, about 99%, about 99.5%, about 99.9%, or any ranges between the specified values. In some embodiments, the substantially pure abiraterone decanoate can be characterized by a purity by weight of about 95%, about 97%, about 99%, about 99.5%, about 99.9%, or any ranges between the specified values. In some embodiments, the substantially pure abiraterone decanoate can also be characterized as having a low palladium content, such as less than 150 ppm, less than 100 ppm, less than 50 ppm, or less than 10 ppm. Abiraterone is typically synthesized with a step of palladium catalyzed cross-coupling reaction. As such, available abiraterone generally has an undesired level of palladium residue, which may be carried into crude abiraterone decanoate product. As described herein, the present disclosure shows that it is possible to reduce the palladium content of abiraterone decanoate to less than 5 ppm, particularly, 3.7 ppm in Example 1B, by using a process of recrystallization with acetone and water as solvents and activated carbon. In some embodiments, the substantially pure abiraterone decanoate conforms to the specification shown in Table D herein (see Example 1B). In some embodiments, the substantially pure abiraterone decanoate comprises an impurity derived from ethyl prasterone. For example, in some embodiments, the substantially pure abiraterone decanoate comprises ethyl prasterone decanoate having the formula:

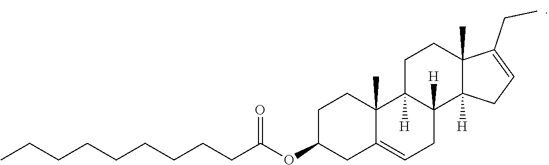

Typically, when present, the substantially pure abiraterone decanoate comprises the ethyl prasterone decanoate in an amount of less than 2% by weight, such as less than 1% by weight, less than 0.5% by weight, such as less than 0.3%, less than 0.2%, or less than 0.1% by weight. The amount of ethyl prasterone decanoate can be readily determined by HPLC methods, such as those descried herein. In some embodiments, the substantially pure abiraterone decanoate can also contain no detectable amount of ethyl prasterone decanoate. Abiraterone starting material is readily available from commercial sources in high purity. Abiraterone starting material obtained from a process using

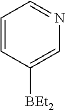

in a cross-coupling reaction to introduce the 3-pyridyl group in abiraterone may contain small amount of impurities which can ultimately be converted into ethyl prasterone. In some embodiments, the substantially pure abiraterone decanoate can be prepared from an abiraterone starting material which has no detectable amount of ethyl prasterone, e.g., those obtained from processes that do not include a cross-coupling with

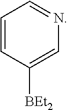

The substantially pure abiraterone decanoate can be in a solid form, such as a crystalline form as described herein. For example, in some embodiments, the substantially pure abiraterone decanoate can be in a crystalline Form A, which can be characterized by an X-Ray Power Diffraction (XRPD) spectrum having one or more (e.g., 1, 2, 3, 4, 5, 6, 7, 8, or 9) of the following peaks: 4.6, 6.9, 8.7, 17.5, 18.3, 18.6, 19.1, 19.6, and 20.8, degrees 2 theta, ±0.2°; a Differential Scanning Calorimetry (DSC) pattern having an endothermic peak with an onset temperature at about 69.0° C.; or a combination thereof. An exemplary procedure is also shown in Example 1A.

In some embodiments, the pharmaceutical composition comprising the substantially pure abiraterone decanoate in its basic form, which is dispersed or dissolved in a pharmaceutically acceptable carrier comprising a pharmaceutically acceptable oil (e.g., described herein) and optionally a further pharmaceutically acceptable solvent (e.g., described herein). In some embodiments, the pharmaceutically acceptable oil comprises a triglyceride (e.g., long and/or medium chain triglycerides), and the further pharmaceutically acceptable solvent, if present, comprises an alcohol, ester, and/or acid solvent. In some embodiments, the pharmaceutically acceptable oil is selected from vegetable oil, castor oil, corn oil, sesame oil, cottonseed oil, peanut oil, poppy seed oil, tea seed oil, and soybean oil, and the further pharmaceutically acceptable solvent, if present, comprises benzyl alcohol, benzyl benzoate, or a combination thereof. In some embodiments, the pharmaceutically acceptable carrier comprises corn oil, benzyl alcohol, and benzyl benzoate.

In some specific embodiments, the pharmaceutical composition comprises the substantially pure abiraterone decanoate in its basic form, which is dissolved in a pharmaceutically acceptable oil (e.g., described herein), benzyl alcohol, and benzyl benzoate. In some embodiments, the pharmaceutical composition comprises the substantially pure abiraterone decanoate in its basic form, which is dissolved in corn oil, benzyl alcohol, and benzyl benzoate. In some embodiments, the pharmaceutical composition comprises: (a) the substantially pure abiraterone decanoate in its basic form, at a concentration of about 25 mg/ml to about 500 mg/ml (e.g., about 25 mg/ml, about 50 mg/ml, about 100 mg/ml, about 120 mg/ml, about 150 mg/ml, about 180 mg/ml, about 200 mg/ml, about 250 mg/ml, about 300 mg/ml, about 400 mg/ml, about 500 mg/ml, or any ranges between the recited values, such as about 100 mg/ml to about 300 mg/ml); (b) benzyl alcohol in an amount of about 50 mg to about 150 mg/mL; (c) benzyl benzoate in an amount of about 100 mg to about 300 mg/mL; and (d) a pharmaceutically acceptable oil (e.g., described herein), for example, corn oil, e.g., q.s. to the volume of the pharmaceutical composition. In some specific embodiments, the pharmaceutical composition comprises, for each milliliter, (a) the substantially pure abiraterone decanoate in its basic form, in an amount of about 100 mg to about 300 mg (e.g., about 100 mg, about 120 mg/ml, about 150 mg, about 180 mg/ml, about 200 mg or about 250 mg, or any ranges between the recited values); (b) benzyl alcohol in an amount of about 50 mg to about 150 mg (e.g., about 75 mg, about 100 mg, or about 125 mg, or any ranges between the recited values); (c) benzyl benzoate in an amount of about 100 mg to about 300 mg (e.g., about 100 mg, about 150 mg, about 200 mg, or about 250 mg, or any ranges between the recited values); and (d) corn oil, q.s. to 1 milliliter. In some embodiments, the weight ratio of benzyl alcohol to benzyl benzoate in the pharmaceutical composition ranges from about 2:1 to about 1:5 (e.g., about 1:1 to 1:3, such as about 1:2). In some embodiments, the pharmaceutical composition can be prepared by mixing (e.g., dissolving) the substantially pure abiraterone decanoate with the pharmaceutically acceptable carrier. For example, in some embodiments, the pharmaceutical composition can be prepared by mixing (e.g., dissolving) the substantially pure abiraterone decanoate in its basic form with corn oil, benzyl alcohol, and benzyl benzoate.

In some embodiments, the pharmaceutical composition comprises the substantially pure abiraterone decanoate in its basic form, which is dissolved in a pharmaceutically acceptable oil (e.g., described herein), benzyl alcohol, and benzyl benzoate, and a pharmaceutically acceptable antioxidant. In some embodiments, the pharmaceutical composition comprises the substantially pure abiraterone decanoate in its basic form, which is dissolved in corn oil, benzyl alcohol, and benzyl benzoate, and 3-mercapto-1,2-propanediol. In some embodiments, the pharmaceutical composition comprises an abiraterone decanoate solution, wherein each milliliter of the abiraterone decanoate solution comprises, consists essentially of, or consists of: (a) the substantially pure abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 20 mg (e.g., about 0.5 mg, about 1 mg, about 2 mg, or about 5 mg); and (c) corn oil, q.s. to 1 milliliter, wherein the abiraterone decanoate has the structure of:

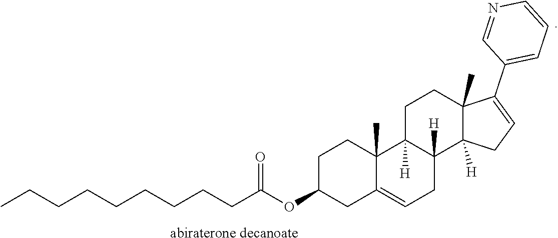

In some embodiments, each milliliter of the abiraterone decanoate solution herein comprises, consists essentially of, or consists of: (a) the substantially pure abiraterone decanoate in its basic form, in an amount of about 180 mg; (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 2 mg (e.g., about 0.5 mg, about 1 mg, or about 2 mg); and (e) corn oil, q.s. to 1 milliliter. The weight ratio of benzyl alcohol to benzyl benzoate in the abiraterone decanoate solution typically ranges from about 2:1 to about 1:5 (e.g., about 1:1 to 1:3, such as about 1:2).

The pharmaceutical composition comprising the substantially pure abiraterone decanoate is typically formulated for parenteral administration. For example, in some embodiments, the pharmaceutical composition is formulated for an intramuscular injection, intradermal injection, or subcutaneous injection, e.g., with a desirable viscosity, glide force, number of particulates, endotoxins, etc. In some embodiments, the pharmaceutical composition is characterized as having (1) a viscosity of less than 0.1 Pa*s, such as about 0.05 Pa*s or lower; (2) a glide force of about 1-10 N when measured using a 21G, 1.5 inch needle, and/or about 2-15 N when measured using a 23 gauge (or 23G), 1.5 inch needle, and/or about 30-150 N when measured using a 27G, 1.5 inch needle; (3) no more than 1000 particles having a size of 10 m or greater, and no more than 300 particles having a size of m or greater, when measured according to USP <788> and/or <789>; and/or (4) less than 100 EU/ml, such as less than 25 EU/ml of bacterial endotoxins measured according to USP <85>. Methods for measuring viscosity and glide force are known in the art, which are also exemplified in Example 2 herein. Glide force measurements can be taken using a 5-mL fill for a 5 mL syringe or 2-mL fill for a 3 mL syringe. The USP methods <788>, <789> and <85> referenced herein should be understood as the current version of such methods, which are also known by those skilled in the art.

In any of the embodiments described herein, unless otherwise specified or contrary from context, the pharmaceutical composition (which may be alternatively referred to as abiraterone prodrug formulation) comprising abiraterone decanoate can be any of the pharmaceutical compositions comprising the substantially pure abiraterone decanoate as described herein.

The abiraterone decanoate in the pharmaceutical composition is typically included in a therapeutically effective amount for treating a disease or disorder described herein, such as prostate cancer. In some embodiments, the abiraterone decanoate can be present in the pharmaceutical composition in an amount sufficient to provide a therapeutically effective blood plasma concentration of abiraterone for a period of at least one week, e.g., at least two weeks, at least four weeks, and up to six or eight weeks or more, such as up to ten weeks or more after a single administration to a human subject having a sex hormone-dependent or androgen receptor driven disease or disorder, such as a sex hormone-dependent benign or malignant disorder, an androgen receptor driven cancer and/or a syndrome due to androgen excess. In some embodiments, the abiraterone decanoate can be present in the pharmaceutical composition in an amount sufficient to provide a therapeutically effective blood plasma concentration of abiraterone at about 1 ng/ml or higher, such as about 2 ng/ml or higher, about 4 ng/ml or higher, about 5 ng/ml or higher, about 8 ng/ml or higher, etc. for a period of at least one week, e.g., at least two weeks, at least four weeks, and up to six or eight weeks or more, such as up to ten weeks or more after a single administration to a human subject having a sex hormone-dependent or androgen receptor driven disease or disorder, such as a sex hormone-dependent benign or malignant disorder, an androgen receptor driven cancer, and/or a syndrome due to androgen excess. In some embodiments, the abiraterone decanoate can be present in the pharmaceutical composition in an amount sufficient to provide a therapeutically effective blood plasma concentration of abiraterone at about 0.5 ng/ml or higher for a period of at least four weeks, e.g., at least six weeks and up to eight weeks or more, such as up to ten weeks or more, after a single administration to a human subject having a sex hormone-dependent or androgen receptor driven disease or disorder, such as a sex hormone-dependent benign or malignant disorder, an androgen receptor driven cancer, and/or a syndrome due to androgen excess. In some embodiments, the abiraterone decanoate can be present in the pharmaceutical composition in an amount sufficient to provide a therapeutically effective blood plasma concentration of abiraterone at about 0.1 ng/ml or higher for a period of at least four weeks, e.g., at least six weeks and up to eight weeks or more, such as up to ten weeks or more, after a single administration to a human subject having a sex hormone-dependent or androgen receptor driven disease or disorder, such as a sex hormone-dependent benign or malignant disorder, an androgen receptor driven cancer, and/or a syndrome due to androgen excess.

The pharmaceutical composition herein is typically in a unit dosage form, which refers to a dosage form in discrete units, such as vials, ampules, pre-filled syringes, etc., with each containing a predetermined amount of the active compound(s). Typically, each unit dosage form herein is for a single use, wherein substantially all the included active compound(s) would be administered to or taken by a subject at one time. For clarity, one or more unit dosage forms can be administered to a subject at each dosing event to satisfy a desired dose of the active compound(s). For example, a pre-filled syringe may be typically used for a single injection to inject substantially all the pre-filled solution or suspension to a subject at one time, and one or more of such pre-filled syringes, which may have the same or different amounts of the active compound(s), may be administered to the subject to satisfy a desired dose of the active compound(s) at each dosing event. For example, for a dosing regimen of 1800 mg abiraterone decanoate every three months, each dosing event requires a total dose of 1800 mg of abiraterone decanoate, which can be satisfied by administering two unit dosage forms each having 900 mg of abiraterone decanoate.

In some embodiments, the present disclosure provides a unit dosage form comprising about 1 milliliter (ml) to about 20 ml of the abiraterone decanoate solution described herein (e.g., those recited in [93]-[103] shown in the Summary section herein). In some embodiments, the unit dosage form can comprise about 1 ml of the abiraterone decanoate solution. In some embodiments, the unit dosage form can comprise about 2 ml of the abiraterone decanoate solution. In some embodiments, the unit dosage form can comprise about 3 ml of the abiraterone decanoate solution. In some embodiments, the unit dosage form can comprise about 4 ml to about 10 ml (e.g., about 4 ml, about 5 ml, about 6 ml, about 7 ml, about 8 ml, about 9 ml, about 10 ml, or any range or value between the recited values) of the abiraterone decanoate solution.

In some specific embodiments, the present disclosure provides a unit dosage form comprising about 1-8 milliliters, such as about 1, 2, 3, 4, 5, 6, 7, or 8 milliliters, of the abiraterone decanoate solution herein, for example, in some embodiments, each milliliter of the abiraterone decanoate solution comprises, consists essentially of, or consists of: (a) abiraterone decanoate in its basic form, in an amount of about 180 mg; (b) benzyl alcohol in an amount of about 100 mg; (c) benzyl benzoate in an amount of about 200 mg; (d) 3-mercapto-1,2-propanediol in an amount of about 0.5 mg to about 2 mg (e.g., about 0.5 mg, about 1 mg, or about 2 mg); and (e) corn oil, q.s. to 1 milliliter. In some embodiments, the abiraterone decanoate solution of the unit dosage form is included in a container having a headspace, such as a vial, ampule, bottle, etc., wherein the headspace is filled with an inert gas, such as nitrogen gas.

In any of the embodiments herein, the pharmaceutical compositions, such as the unit dosage forms herein, can also be characterized as being storage stable at room temperature, for example, being stable when stored at room temperature (25±2° C.) at 60% relative humidity (RH) ±5% RH, for about 1 month, 2 months, 3 months, 6 months, 9 months or longer. By storage stable, it is to be meant that the pharmaceutical compositions would be accepted by those skilled in the art as equivalent to the initial pharmaceutical compositions, i.e., at the beginning of the storage. Storage stable is typically characterized by one or more of the following: (1) substantially same amount of drug related impurities, no significant increased amount of either individual or total impurities; (2) substantially same amount of abiraterone decanoate; and (3) substantially same physical properties such as viscosity, physical states, color, etc. “Substantially same” should be understood as meaning within 80-125% or measurement error margin.

In some specific embodiments, the present disclosure provides a pharmaceutical composition, e.g., unit dosage form, comprising abiraterone decanoate having the formula of:

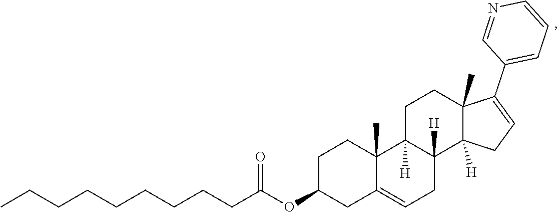

a pharmaceutically acceptable oil, and a pharmaceutically acceptable solvent, wherein the abiraterone decanoate is in its basic form, which is present at a concentration of about 25 mg/ml to about 500 mg/ml, such as about 50 mg/ml, about 100 mg/ml, about 120 mg/ml, about 150 mg/ml, about 180 mg/ml, about 200 mg/ml, about 250 mg/ml, about 300 mg/ml, about 350 mg/ml, about 400 mg/ml, about 500 mg/ml, or any ranges between the recited values, wherein the pharmaceutical composition, e.g., unit dosage form, is formulated for parenteral injection, such as intramuscular injection, intradermal injection, or subcutaneous injection, wherein the pharmaceutical composition, e.g., unit dosage form, comprises the abiraterone decanoate in an amount of about 50 mg to about 5,000 mg, such as about 100 mg, about 350 mg, about 500 mg, about 1000 mg, about 1500 mg, about 2000 mg, about 5000 mg, or any ranges between the recited values. In some embodiments, the pharmaceutical composition can be in a unit dosage form. Typically, depending on the dosing amount, one or more (e.g., 1) of the unit dosage forms can be administered to a human subject in need thereof. For example, in some embodiments, each unit dosage form can include about 180 mg, about 360 mg, about 720 mg, about 1260 mg, or about 1800 mg of abiraterone decanoate, with the concentration of abiraterone decanoate in the formulation being about 180 mg/ml, or about 1 ml, about 2 mL, about 3 mL, about 7 ml, about 10 ml of the formulation, which may be packaged, for example, in a bottle, vial, ampule, or in a pre-filled syringe. The pharmaceutically acceptable oil in the pharmaceutical composition, e.g., unit dosage form, can be any of those described herein. For example, in some embodiments, the pharmaceutically acceptable oil is a pharmaceutically acceptable oil for injection, including oils of vegetable origin or synthetic mono- or diglycerides of fatty acids. In some embodiments, the pharmaceutically acceptable oil can be nature oil, synthetic oil, or semi-synthetic oil, such as fractionated coconut oil and medium-chain triglycerides, such as those sold under the trademark Miglyol. In some embodiments, the pharmaceutically acceptable oil can comprise a triglyceride derived from fatty acids. In some embodiments, the pharmaceutically acceptable oil can comprise a triglyceride derived from long and/or medium chain fatty acids, which can be independently poly-unsaturated, mono-unsaturated, or saturated. In some embodiments, the pharmaceutically acceptable oil can be selected from vegetable oil, castor oil, corn oil, sesame oil, cottonseed oil, peanut oil (arachis oil), poppy seed oil, tea seed oil, and soybean oil. In some specific embodiments, the pharmaceutically acceptable oil can comprise corn oil, which includes a triglyceride, in which the fatty acid constituents are primarily linoleic acid, oleic acid, palmitic acid, and stearic acid. The pharmaceutically acceptable solvent in the pharmaceutical composition, e.g., unit dosage form, also include any of those described herein. In some embodiments, the pharmaceutically acceptable solvent (or co-solvent if the oil is counted as a solvent), such as an alcohol, ester, acid, etc. In some embodiments, the pharmaceutically acceptable solvent can include benzyl alcohol, benzyl benzoate, ethanol, glycerol, polyethylene glycol, polysorbate 80, acetic acid, and/or ethyl acetate. In some embodiments, the pharmaceutically acceptable solvent can be benzyl alcohol and/or benzyl benzoate. In some embodiments, the pharmaceutical composition, e.g., unit dosage form, comprises abiraterone decanoate, a pharmaceutically acceptable oil (e.g., described herein), benzyl alcohol, and benzyl benzoate. In some embodiments, the pharmaceutically acceptable oil is corn oil. In some embodiments, the benzyl alcohol is present in an amount of about 5-10% by volume, the benzyl benzoate is present in an amount of about 10-20% by volume, and corn oil is present in an amount of about 70-85% by volume, with the combined volume of benzyl alcohol, benzyl benzoate, and corn oil being 100%. In some embodiments, the abiraterone decanoate is in a substantially pure form as described herein. In some particular embodiments, the pharmaceutical composition comprises: (a) abiraterone decanoate, such as the substantially pure abiraterone decanoate herein, in its basic form, at a concentration of about 25 mg/ml to about 500 mg/ml (e.g., about 25 mg/ml, about 50 mg/ml, about 100 mg/ml, about 120 mg/ml, about 150 mg/ml, about 180 mg/ml, about 200 mg/ml, about 250 mg/ml, about 300 mg/ml, about 400 mg/ml, about 500 mg/ml, or any ranges between the recited values, such as about 100 mg/ml to about 300 mg/ml); (b) benzyl alcohol in an amount of about 50 mg to about 150 mg/mL; (c) benzyl benzoate in an amount of about 100 mg to about 300 mg/mL; and (d) a pharmaceutically acceptable oil (e.g., described herein), in particular, corn oil, e.g., q.s. to the volume of the pharmaceutical composition. In some specific embodiments, the pharmaceutical composition comprises, for each milliliter, (a) abiraterone decanoate, such as the substantially pure abiraterone decanoate herein, in its basic form, in an amount of about 100 mg to about 300 mg (e.g., about 100 mg, about 150 mg, about 200 mg or about 250 mg, or any ranges between the recited values); (b) benzyl alcohol in an amount of about 50 mg to about 150 mg (e.g., about 75 mg, about 100 mg, or about 125 mg, or any ranges between the recited values); (c) benzyl benzoate in an amount of about 100 mg to about 300 mg (e.g., about 100 mg, about 150 mg, about 200 mg, or about 250 mg, or any ranges between the recited values); and (d) corn oil, q.s. to 1 milliliter. In some embodiments, the weight ratio of benzyl alcohol to benzyl benzoate in the pharmaceutical composition ranges from about 2:1 to about 1:5 (e.g., about 1:1 to 1:3, such as about 1:2). In some specific embodiments, the pharmaceutical composition comprises abiraterone decanoate, benzyl alcohol, benzyl benzoate, and corn oil, each in a respective amount (mg per 1 milliliter) substantially the same as that shown in Example 2 of this disclosure, for example, in some embodiments, the pharmaceutical composition comprises, per 1 milliliter, abiraterone decanoate about 200 mg, benzyl alcohol about 100 mg, benzyl benzoate about 200 mg, and corn oil q.s. to 1 mL; or Abiraterone Decanoate, about 180 mg; Benzyl Alcohol, about 100 mg; Benzyl Benzoate, about 200 mg; and Corn oil, q.s. to 1 mL. In some specific embodiments, the pharmaceutical composition comprises abiraterone decanoate, benzyl alcohol, benzyl benzoate, corn oil, and monothioglycerol, each in a respective amount (mg per 1 milliliter) substantially the same as that shown in Example 4 of this disclosure.

In some embodiments, the present disclosure provides exemplary abiraterone decanoate formulations as shown in Table C. All numeric values in the table should be understood as preceded by the term “about.” The concentration of abiraterone decanoate refers to the amount of abiraterone decanoate in mg per ml of the final formulation, which can be a solution or suspension. The amount of oil (the primary solvent) and co-solvent (benzyl alcohol and/or benzyl benzoate) in the tables is expressed as volume percentage of solvent, which includes both the oil and co-solvent. Suitable oil includes any of the pharmaceutically acceptable oil as described herein, such as corn oil. Optional additional ingredients are not shown in Table C.

TABLE C
Exemplary Abiraterone Decanoate Formulations

| | Amount/Concentration | | |
|---|---|---|---|
| | | | More Exemplary |
| Ingredients | Typical | Exemplary range | Range |
| Abiraterone | 25 mg/ml to 500 | 50 mg/ml to 300 | 75 mg/ml to 300 |
| decanoate | mg/ml | mg/ml; 100 mg/ml to | mg/ml, such as 150 |
| | | 300 mg/ml | mg/ml to about 250 |
| | | | mg/ml |
| Oil (e.g., corn oil, | 30% to 100% of | 50% to 90% of solvent | 60% to 90% of |
| castor oil, sesame oil, | solvent | | solvent, such as 70% |
| peanut oil, cottonseed | | | |
| oil, and/or Miglyol | | | |
| 812) | | | |
| benzyl alcohol | 0% to 20% of | 0% to 15% of solvent | 0% to 10% of solvent, |
| | solvent | | such as 10% |
| benzyl benzoate | 0% to 50% of | 0% to 35% of solvent | 0% to 30% of solvent, |
| | solvent | | such as 20% |

The pharmaceutical composition or unit dosage form herein can be prepared by those skilled in the art in view of the methods disclosed herein. In some embodiments, the present disclosure provides a method for preparing an abiraterone decanoate formulation suitable for parenteral administration to a human subject having a sex hormone-dependent or androgen receptor driven disease or disorder, such as a sex hormone-dependent benign or malignant disorder, an androgen receptor driven cancer, and/or a syndrome due to androgen excess. In some embodiments, the method comprises mixing (such as dissolving or suspending) abiraterone decanoate, which has the formula of:

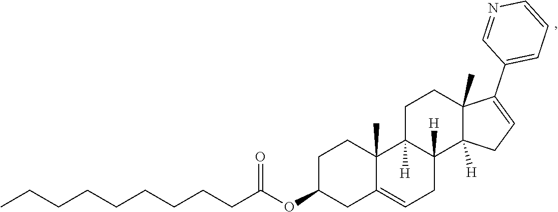

and optionally a pharmaceutically acceptable antioxidant (e.g., monothioglycerol), in a pharmaceutically acceptable carrier to form a mixture (such as a solution or suspension). In some embodiments, the abiraterone decanoate is in a substantially pure form as described herein. In some embodiments, the method further comprises sterilizing the mixture (e.g., solution or suspension), such as by sterilizing filtration. In some embodiments, the method further comprises filling the mixture in a container having a headspace, introducing an inert gas to the headspace of the container, and optionally capping/sealing the container. An exemplary manufacturing process is also described in Example 5 herein. In some embodiments, the container can be a vial, an ampule, or a bottle. In some embodiments, the method further comprises adding the mixture to a syringe. In some embodiments, the dissolving or suspending can comprise mixing (e.g., dissolving or suspending) the crystalline form (e.g., Form A) of abiraterone decanoate described herein in the pharmaceutically acceptable carrier. In some embodiments, the mixing (such as dissolving or suspending) can comprise mixing (e.g., dissolving or suspending) the substantially pure abiraterone decanoate described herein in the pharmaceutically acceptable carrier. Suitable pharmaceutically acceptable carriers and amounts, amount of abiraterone decanoate, concentration of abiraterone decanoate, pharmaceutically acceptable antioxidant (e.g., monothioglycerol), and amounts thereof, include any of those described herein. For example, in some embodiments, the pharmaceutically acceptable carrier comprises a pharmaceutically acceptable oil and a pharmaceutically acceptable solvent, wherein the pharmaceutically acceptable oil comprises a vegetable oil, castor oil, corn oil, sesame oil, cottonseed oil, peanut oil, poppy seed oil, tea seed oil, or soybean oil, the pharmaceutically acceptable solvent comprises benzyl alcohol and/or benzyl benzoate, and wherein the abiraterone decanoate is present at a concentration of about 50 mg/mL to about 300 mg/mL such as about 100 mg/mL to about 300 mg/mL, such as about 180 mg/mL, and wherein the pharmaceutically acceptable antioxidant is monothioglycerol in an amount of about 2 mg/ml or less. In some embodiments, the pharmaceutically acceptable carrier can be a combination of corn oil, benzyl alcohol, and benzyl benzoate, in any amount described herein. In some embodiments, the abiraterone decanoate is present at a concentration of 150 mg/mL to about 250 mg/mL, such as about 180 mg/mL or about 200 mg/mL. In some embodiments, the pharmaceutically acceptable antioxidant is monothioglycerol, which is in an amount of about 0.5 mg/ml, about 1 mg/mL, about 2 mg/mL, or any range between the recited values.

### Abiraterone Prodrug of Formula I

In some embodiments, the pharmaceutical composition can comprise an abiraterone prodrug according to any of those described in U.S. Pat. No. 10,792,292 B2, and PCT Application Nos. PCT/US2021/048607 and PCT/US2022/016278. For example, in some embodiments, the pharmaceutical composition can include an abiraterone prodrug of Formula I, or a pharmaceutically acceptable salt thereof:

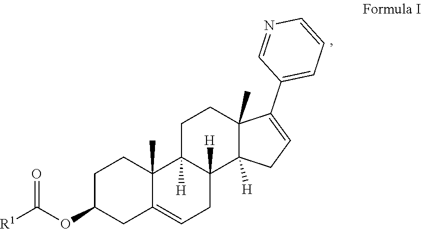

wherein R¹ is R¹⁰, O—R¹⁰, or NHR¹⁰, wherein R¹⁰ is selected from: a C₇₋₃₀ alkyl; C₇₋₃₀ alkenyl; C₇₋₃₀ alkynyl; an alkyl substituted with a cycloalkyl, which typically has a total number of carbons between 5 and 16; an alkyl substituted with a phenyl, which typically has a total number of carbons between 7 and 16; a cycloalkyl optionally substituted with one or more alkyl, which typically has a total number of carbons between 5 and 16; and a branched C5 or C6 alkyl such as

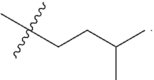 [SMILES: *CCC(C)C]

In some preferred embodiments, R¹⁰ is a C₇₋₃₀ alkyl. As used herein, unless expressly stated to be substituted, an alkyl should be understood as unsubstituted. However, an alkyl can be either linear or branched. In some embodiments, R¹⁰ can be a linear C₇₋₃₀ alkyl. In some embodiments, R¹⁰ can be a branched C₇₋₃₀ alkyl. In some embodiments, R¹⁰ is a linear C₇₋₁₆ alkyl, for example, R¹⁰ can have a formula —(CH₂)ₙ—CH₃, wherein n is an integer between 6 and 15 (e.g., between 6 and 12, such as 6, 7, 8, 9, 10, 11, or 12). In some embodiments, R¹⁰ can be a branched C₇₋₁₆ alkyl.

In some embodiments, R¹⁰ can also be an alkyl substituted with a cycloalkyl. Typically, in such embodiments, R¹⁰ has a total number of carbons between 5 and 16, i.e., the total number of carbons from the alkyl moiety and the cycloalkyl moiety are between 5 and 16. The cycloalkyl typically is unsubstituted. However, in some embodiments, the cycloalkyl can be optionally substituted, e.g., with one or two lower alkyl (e.g, a C₁₋₄ alkyl). In some embodiments, R¹⁰ can be an alkyl substituted with a C₃₋₆ cycloalkyl, which typically has a total number of carbons between 6 and 12. In some embodiments, R¹⁰ can be a linear alkyl substituted with a C₃₋₆ cycloalkyl, for example, R¹⁰ can have a formula —(CH₂)ₙ-Cy, wherein n is an integer of 1-6 (e.g., 1, 2, 3, 4, 5, or 6), and Cy is a C₃₋₆ cycloalkyl (such as cyclopropyl, cyclobutyl, cyclopentyl, or cyclohexyl). In some embodiments, R¹⁰ can have a formula —(CH₂)ₙ-Cy, wherein n is 1 or 2, and Cy is cyclopentyl or cyclohexyl. In some embodiments, R¹⁰ can also be a branched alkyl (e.g., branched C₂-6) substituted with a C₃₋₆ cycloalkyl. As used herein, a branched C2 alkyl should be understood as a 1,1-disubstituted ethyl group, for example, —CH(CH₃)-Cy.

In some embodiments, R¹⁰ can also be an alkyl substituted with a phenyl. Typically, in such embodiments, R¹⁰ has a total number of carbons between 7 and 16, i.e., the total number of carbons from the alkyl moiety and the phenyl moiety are between 5 and 16. In some embodiments, R¹⁰ can be a linear alkyl substituted with a phenyl, for example, R¹⁰ can have a formula —(CH₂)ₙ-Cy, wherein n is an integer of 1-6 (e.g., 1, 2, 3, 4, 5, or 6), and Cy is a phenyl. In some embodiments, R¹⁰ can have a formula —(CH₂)ₙ-Cy, wherein n is 1 or 2, and Cy is phenyl. In some embodiments, R¹⁰ can also be a branched alkyl (e.g., branched C₂-6) substituted with a phenyl. The phenyl typically is unsubstituted. However, in some embodiments, the phenyl can be optionally substituted, e.g., with one or two lower alkyl (e.g, a C₁₋₄ alkyl).

In some embodiments, R¹⁰ can be a cycloalkyl optionally substituted with one or more alkyl. In such embodiments, R¹⁰ typically has a total number of carbons between 5 and 16, i.e., the total number of carbons of the cycloalkyl and its optional substituents are between 5 and 16. In some embodiments, R¹⁰ can be a C₃₋₆ cycloalkyl, either unsubstituted or substituted with a C₁₋₄ alkyl. In some specific embodiments, R¹⁰ can be

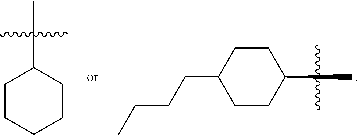

In some embodiments, R¹⁰ can be a branched C5 or C6 alkyl. In some embodiments, R¹⁰ can be

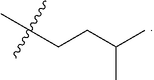 [SMILES: *CCC(C)C]

Other branched C5 or C6 alkyls are also suitable.

In some embodiments, R¹⁰ can be an unsaturated aliphatic group such as a C₇₋₃₀ alkenyl or a C₇₋₃₀ alkynyl.

In some preferred embodiments, the compound of Formula I is an ester of abiraterone, e.g., R¹ is R¹⁰, wherein R¹⁰ is defined herein. In some embodiments, R¹ in Formula I can be a C₇₋₁₆ alkyl, e.g., an alkyl having a formula of —(CH₂)ₙ—CH₃, wherein n is an integer between 6 and 12 (e.g., 6, 7, 8, 9, 10, 11, or 12). In some embodiments, R¹ in Formula I can be represented by the formula —(CH₂)ₙ-Cy, wherein n is an integer of 1-6, and Cy is a C₃₋₆ cycloalkyl or phenyl, for example, in more specific embodiments, n can be 1 or 2, and Cy is cyclopentyl, cyclohexyl, or phenyl. In some specific embodiments, R¹ in Formula I can be

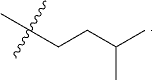 [SMILES: *CCC(C)C]

In some specific embodiments, R¹ in Formula I can be

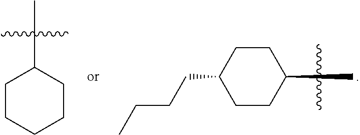

Other suitable groups for R¹ include any of the R¹⁰ defined herein.

In some embodiments, R¹ in Formula I can also be O—R¹⁰ or NHR¹⁰, wherein R¹⁰ is defined herein.

Typically, compounds of Formula I can be present in a formulation in the basic form, for example, in a non-aqueous formulation. However, in some embodiments, pharmaceutically acceptable salts of compounds of Formula I are also useful. Unless specifically referred to as in its salt form or otherwise contradictory from context, the compound of Formula I can be in its basic form in the abiraterone prodrug formulations described herein. In some embodiments, the compound of Formula I can be in a substantially pure form.

### Abiraterone Prodrug Formulations

In some embodiments, the pharmaceutical composition comprising the abiraterone prodrug can be an abiraterone prodrug formulation according to any of those described in U.S. Pat. No. 10,792,292 B2, and PCT Application Nos. PCT/US2021/048607 and PCT/US2022/016278, which can be used for the method herein. Typically, the abiraterone prodrugs can be formulated as a parenteral formulation, such as an intramuscular, intradermal, or subcutaneous formulation, and can in some embodiments be formulated to deliver a therapeutically effective plasma concentration of abiraterone over an extended period of time, e.g., for at least 1 week, at least 2 weeks, at least 3 weeks, at least 4 weeks, and up to six or eight weeks or more, such as up to ten weeks or more, etc.

Various abiraterone prodrugs, such as abiraterone esters, carbamates, or carbonates are suitable for compositions and methods of the present disclosure. In some embodiments, the pharmaceutical composition can comprise a compound of Formula I (e.g., any one or more as defined herein), or a pharmaceutically acceptable salt thereof. In some embodiments, the pharmaceutical composition can be formulated for parenteral administration, such as intramuscular injection, intradermal injection, or subcutaneous injection. The pharmaceutical composition typically includes a pharmaceutically acceptable carrier. Suitable carriers include those known in the art, for example, those described in “Remington: The Science and Practice of Pharmacy” (formerly “Remington's Pharmaceutical Sciences,” University of the Sciences in Philadelphia, Lippincott, Williams & Wilkins, Philadelphia, Pa. (2005)) and the U.S. Food and Drug Administration's Center for Drug Evaluation and Research's database of inactive ingredients present in FDA-approved drugs. In some embodiments, the pharmaceutically acceptable carrier can be a carrier that is approved for use by the FDA for an intramuscular, intradermal, or subcutaneous drug product, e.g., those listed in the FDA's database of inactive ingredients. In some embodiments, the pharmaceutically acceptable carrier can be any suitable nonaqueous vehicle suitable for injection, such as those described in U.S. Pharmacopeia. In some embodiments, the pharmaceutically acceptable carrier can be a pharmaceutically acceptable oil, e.g., a vegetable oil, castor oil, corn oil, sesame oil, cottonseed oil, peanut oil, poppy seed oil, tea seed oil, or soybean oil. In some embodiments, the pharmaceutically acceptable oil can be oils (e.g., described herein) suitable for use as vehicles for injection, e.g., meeting the criteria as described in the corresponding U.S. Pharmacopeia monograph. In some embodiments, the pharmaceutically acceptable oil can be an oil of vegetable origin suitable for use as vehicles for injection. In some embodiments, the pharmaceutically acceptable oil can be a synthetic oil suitable for use as vehicles for injection, such as a synthetic mono- or diglycerides of fatty acids, e.g., those that are liquid and remain clear when cooled to 10° C. and have an Iodine Value of not more than 140. In some embodiments, the pharmaceutically acceptable oil can be nature oil, synthetic oil, or semi-synthetic oil, such as fractionated coconut oil and medium-chain triglycerides, such as those sold under the trademark Miglyol. In some embodiments, the pharmaceutically acceptable carrier comprises a triglyceride derived from fatty acids. In some embodiments, the pharmaceutically acceptable carrier comprises a triglyceride derived from long and/or medium chain fatty acids, which can be independently poly-unsaturated, mono-unsaturated, or saturated. In some embodiments, two or more different pharmaceutically acceptable oil can be used. In some embodiments, the pharmaceutical composition is a non-aqueous solution or suspension. In some embodiments, the pharmaceutical composition further comprises a pharmaceutically acceptable solvent, such as benzyl alcohol, benzyl benzoate, or a combination thereof. In some embodiments, the compound of Formula I or pharmaceutically acceptable salt thereof can be present in the pharmaceutical composition at a concentration of about 25 mg/ml to about 500 mg/ml (e.g., about 25 mg/ml, about 50 mg/ml, about 100 mg/ml, about 150 mg/ml, about 200 mg/ml, about 250 mg/ml, about 300 mg/ml, about 400 mg/ml, about 500 mg/ml, or any ranges between the recited values).

In some embodiments, the pharmaceutical composition suitable for use in the method herein can comprise a compound of Formula II, or a pharmaceutically acceptable salt thereof.

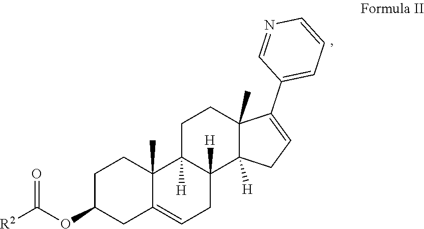

wherein R² is defined herein. In some embodiments, the pharmaceutical composition can be formulated for intramuscular injection, intradermal injection, or subcutaneous injection. In some embodiments, the compound of Formula II or pharmaceutically acceptable salt thereof can be present in the pharmaceutical composition at a concentration of about 25 mg/ml to about 500 mg/ml (e.g., about 25 mg/ml, about 50 mg/ml, about 100 mg/ml, about 150 mg/ml, about 200 mg/ml, about 250 mg/ml, about 300 mg/ml, about 400 mg/ml, about 500 mg/ml, or any ranges between the recited values). In some embodiments, the pharmaceutical composition is a non-aqueous solution or suspension. In some embodiments, the compound of Formula II or pharmaceutically acceptable salt thereof is dissolved or suspended in a pharmaceutically acceptable oil (e.g., described herein), such as a vegetable oil, castor oil, corn oil, sesame oil, cottonseed oil, peanut oil, poppy seed oil, tea seed oil, or soybean oil. In some embodiments, the pharmaceutical composition further comprises a pharmaceutically acceptable solvent, such as benzyl alcohol, benzyl benzoate, or a combination thereof.

Various groups are suitable as R² in Formula II. In some embodiments, R² can be selected such that the compound of Formula II is an ester, a carbamate, or a carbonate of abiraterone. In some embodiments, R² is R²⁰, O—R²⁰, or NHR²⁰, and R²⁰ is selected from: a C₁₋₃₀ alkyl; a C₂₋₃₀ alkenyl; a C₂₋₃₀ alkynyl; an alkyl substituted with a cycloalkyl, which typically has a total number of carbons between 4 and 30; an alkyl substituted with a phenyl, which typically has a total number of carbons between 7 and 30; and a cycloalkyl optionally substituted with one or more alkyl, which typically has a total number of carbons between 3 and 30.

In some preferred embodiments, R²⁰ is a C₁₋₁₆ alkyl. In some embodiments, R²⁰ can be a linear C₁₋₁₆ alkyl. In some embodiments, R²⁰ can be a branched C₃₋₁₆ alkyl. In some embodiments, R²⁰ can be a branched C5 or C6 alkyl. In some embodiments, R²⁰ can be

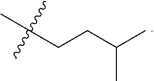 [SMILES: *CCC(C)C]

In some embodiments, R²⁰ can have a formula —(CH₂)ₙ—CH₃, wherein n is an integer between 0 and 12 (e.g., between 6 and 12, such as 6, 7, 8, 9, 10, 11, or 12).

In some embodiments, R²⁰ can also be an alkyl substituted with a cycloalkyl. Typically, in such embodiments, R²⁰ has a total number of carbons between 4 and 30, such as between 5 and 16 (i.e., the total number of carbons from the alkyl moiety and the cycloalkyl moiety are between 5 and 16). The cycloalkyl typically is unsubstituted. However, in some embodiments, the cycloalkyl can be optionally substituted, e.g., with one or two lower alkyl (e.g, a C₁₋₄ alkyl). In some embodiments, R²⁰ can be an alkyl substituted with a C₃₋₆ cycloalkyl, which typically has a total number of carbons between 6 and 12. In some embodiments, R²⁰ can be a linear alkyl substituted with a C₃₋₆ cycloalkyl, for example, R²⁰ can have a formula —(CH₂)ₙ-Cy, wherein n is an integer of 1-6 (e.g., 1, 2, 3, 4, 5, or 6), and Cy is a C₃₋₆ cycloalkyl (such as cyclopropyl, cyclobutyl, cyclopentyl, or cyclohexyl). In some embodiments, R²⁰ can have a formula —(CH₂)ₙ-Cy, wherein n is 1 or 2, and Cy is cyclopentyl or cyclohexyl. In some embodiments, R²⁰ can also be a branched alkyl (e.g., branched C₂-6) substituted with a C₃₋₆ cycloalkyl.

In some embodiments, R²⁰ can also be an alkyl substituted with a phenyl. Typically, in such embodiments, R²⁰ has a total number of carbons between 7 and 30, e.g., between 7 and 16 (i.e., the total number of carbons from the alkyl moiety and the phenyl moiety are between 7 and 16). In some embodiments, R²⁰ can be a linear alkyl substituted with a phenyl, for example, R²⁰ can have a formula —(CH₂)ₙ-Cy, wherein n is an integer of 1-6 (e.g., 1, 2, 3, 4, 5, or 6), and Cy is a phenyl. In some embodiments, R²⁰ can have a formula —(CH₂)ₙ-Cy, wherein n is 1 or 2, and Cy is phenyl. In some embodiments, R²⁰ can also be a branched alkyl (e.g., branched C₂-6) substituted with a phenyl. The phenyl typically is unsubstituted. However, in some embodiments, the phenyl can be optionally substituted, e.g., with one or two lower alkyl (e.g, a C₁₋₄ alkyl).

In some embodiments, R²⁰ can be a cycloalkyl optionally substituted with one or more alkyl. In such embodiments, R²⁰ typically has a total number of carbons between 3 and 30, e.g., 5 and 16 (i.e., the total number of carbons of the cycloalkyl and its optional substituents are between 5 and 16). In some embodiments, R²⁰ can be a C₃₋₆ cycloalkyl, either unsubstituted or substituted with a C₁₋₄ alkyl. In some specific embodiments, R²⁰ can be

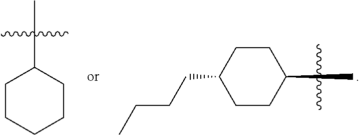

In some embodiments, R²⁰ can be an unsaturated aliphatic group such as a C₂₋₃₀ alkenyl or a C₂₋₃₀ alkynyl.

In some preferred embodiments, the compound of Formula II is an abiraterone ester, e.g., R² is R²⁰, wherein R²⁰ is defined herein. In some embodiments, R² in Formula II can be a C₁₋₁₆ alkyl, e.g., an alkyl having a formula of —(CH₂)ₙ—CH₃, wherein n is an integer between 0 and 12. In some embodiments, R² in Formula II can be represented by the formula —(CH₂)ₙ-Cy, wherein n is an integer of 1-6, and Cy is a C₃₋₆ cycloalkyl or phenyl, for example, in more specific embodiments, n can be 1 or 2, and Cy is cyclopentyl, cyclohexyl, or phenyl. In some specific embodiments, R² in Formula II can be

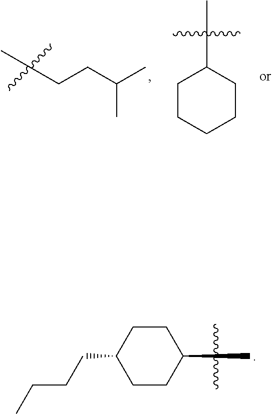

Other suitable groups for R² include any of the R²⁰ defined herein. In some embodiments, the abiraterone ester can be an acetate, a propionate, a butanoate, a (vaterate) pentanoate, an isocaproate, a buciclate, a cyclohexanecarboxylate, a phenyl propionate, caproate (hexanoate), an enanthate (heptanoate), a cypionate, an octanoate, a noncanoate, a decanoate, an undecanoate, a dodecanoate, a tridecanoate, a tetradecanoate, a pentadecanoates, or a hexadecanoate of abiraterone. In some embodiments, the abiraterone ester can be abiraterone acetate, abiraterone propionate, and abiraterone decanoate. In some specific embodiments, the abiraterone ester can be abiraterone pentanoate, abiraterone hexanoate, abiraterone heptanoate, abiraterone decanoate, abiraterone isocaproate, or abiraterone cypionate.

In some embodiments, R² in Formula II can also be O—R²⁰ or NHR²⁰, wherein R²⁰ is defined herein.

Typically, compounds of Formula II can be present in a formulation in the basic form, for example, in a non-aqueous formulation. However, in some embodiments, pharmaceutically acceptable salts of compounds of Formula II are also useful. Unless specifically referred to as in its salt form or otherwise contradictory from context, the compound of Formula II can be in its basic form in the abiraterone prodrug formulations described herein. In some embodiments, the compound of Formula II can be in a substantially pure form.

The pharmaceutical composition comprising the substantially pure compound of Formula I or II (e.g., abiraterone decanoate) is typically formulated for parenteral administration. For example, in some embodiments, the pharmaceutical composition is formulated for an intramuscular injection, intradermal injection, or subcutaneous injection, e.g., with a desirable viscosity, glide force, number of particulates, endotoxins, etc. In some embodiments, the pharmaceutical composition is characterized as having (1) a viscosity of less than 0.1 Pa*s, such as about 0.05 Pa*s or lower; (2) a glide force of about 1-10 N when measured using a 21G, 1.5 inch needle, and/or about 2-15 N when measured using a 23 gauge (or 23G), 1.5 inch needle, and/or about 30-150 N when measured using a 27G, 1.5 inch needle; (3) no more than 1000 particles having a size of 10 m or greater, and no more than 300 particles having a size of 25 m or greater, when measured according to USP <788> and/or <789>; and/or (4) less than 100 EU/ml, such as less than 25 EU/ml of bacterial endotoxins measured according to USP <85>. Methods for measuring viscosity and glide force are known in the art, which are also exemplified in Example 2 herein. The USP methods <788>, <789> and <85> referenced herein should be understood as the current version of such methods, which are also known by those skilled in the art.

Typically, the abiraterone prodrugs of the present disclosure are formulated as a non-aqueous solution or suspension. In some embodiments, the non-aqueous solution or suspension provides higher levels of abiraterone in the plasma for a longer duration, when compared to an aqueous solution or suspension. For example, as detailed herein, IM injections of an aqueous suspension and a vegetable oil solution of the abiraterone acetate prodrug were evaluated in rats. Surprisingly, it was determined that the vegetable oil solution (but not the aqueous suspension) of abiraterone acetate prodrug gave the highest blood plasma levels and the longest duration of exposure of active drug abiraterone. Accordingly, in some embodiments, the abiraterone prodrug formulations herein can include an abiraterone prodrug of the present disclosure (e.g., compound of Formula I or II) dissolved or dispersed in a pharmaceutically acceptable carrier. In some embodiments, the pharmaceutically acceptable carrier can be any suitable nonaqueous vehicle suitable for injection, such as those described in U.S. Pharmacopeia. In some embodiments, the pharmaceutically acceptable carrier can be a pharmaceutically acceptable oil, e.g., a vegetable oil, castor oil, corn oil, sesame oil, cottonseed oil, peanut oil, poppy seed oil, tea seed oil, or soybean oil. In some embodiments, the pharmaceutically acceptable oil can be oils (e.g., described herein), suitable for use as vehicles for injection, e.g., meeting the criteria as described in the corresponding U.S. Pharmacopeia monograph. In some embodiments, the pharmaceutically acceptable oil can be an oil of vegetable origin suitable for use as vehicles for injection. In some embodiments, the pharmaceutically acceptable oil can be a synthetic oil suitable for use as vehicles for injection, such as synthetic mono- or diglycerides of fatty acids, e.g., those that are liquid and remain clear when cooled to 10° C. and have an Iodine Value of not more than 140. In some embodiments, the pharmaceutically acceptable oil can be nature oil, synthetic oil, or semi-synthetic oil, such as fractionated coconut oil and medium-chain triglycerides, such as those sold under the trademark Miglyol. In some embodiments, the pharmaceutically acceptable carrier comprises a triglyceride derived from fatty acids. In some embodiments, the pharmaceutically acceptable carrier comprises a triglyceride derived from long and/or medium chain fatty acids, which can be independently poly-unsaturated, mono-unsaturated, or saturated. In some embodiments, the pharmaceutically acceptable oil can be any of those that are approved for use by the FDA for an intramuscular, intradermal, or subcutaneous drug product, e.g., those listed in the FDA's database of inactive ingredients. In some specific embodiments, the pharmaceutically acceptable oil is castor oil or corn oil. In some embodiments, two or more different pharmaceutically acceptable oil can be used.

Other ingredients can also be optionally included in the abiraterone prodrug formulations herein. In some embodiments, the abiraterone prodrug formulation can further comprise a pharmaceutically acceptable solvent, such as benzyl alcohol, benzyl benzoate, ethanol, glycerol, polyethylene glycol, polysorbate 80, acetic acid, and ethyl acetate. It was determined that the additives/co-solvents benzyl alcohol and benzyl benzoate had the advantage of increasing the solubility of the prodrugs as well as reducing the viscosity and/or glide force of the solution, see e.g., U.S. Pat. No. 10,792,292 B2, and PCT Application Nos. PCT/US2021/048607 and PCT/US2022/016278, which provided a more concentrated solution that was easier to inject through an acceptable gauge needle for IM injection (e.g., 20-27 gauge such as 22-25 gauge). The co-solvent can be selected based on its ability to reduce the viscosity of the vehicle to allow injection through suitable injection needles or cannula. Benzyl alcohol as an additive in IM or subcutaneous injections also has the advantage that it can act as a local anesthetic at the injection site (Wilson et al. Ann. Emer. Med. 33(5), 495, 1999). In some embodiments, the abiraterone prodrug formulation further comprises benzyl alcohol. In some embodiments, the cosolvent, if present, can be included at a level (e.g., about 0-50% of the solvent, such as about 10%) such that it does not cause irritation (or only minimal or tolerable irritation) at the injection site.

In some embodiments, the abiraterone prodrug formulation can comprise benzyl benzoate as a cosolvent, for example, about 0-50% of the solvent, typically 0-35% or 0-30%, or about 20%. In some embodiments, the abiraterone prodrug formulation can comprise a combination of benzyl alcohol and benzyl benzoate as cosolvents. In some embodiments, the benzyl alcohol can be present in an amount of about 0-20% (e.g., 0-15% or 0-10%, such as about 10%) of the solvent, and benzyl benzoate can be present in an amount of about 0-50% (e.g., 0-35% or 0-30%, such as about 20%) of the solvent, wherein the balance of the solvent can be any one or more of the pharmaceutically acceptable oil described herein, such as corn oil, castor oil, sesame oil, peanut oil, cottonseed oil, and/or Miglyol 812, etc. The combination of benzyl alcohol and benzyl benzoate were shown to achieve a lower viscosity and glide force, when compared with using just benzyl alcohol or benzyl benzoate. Further, it was unexpectedly found that a representative abiraterone prodrug (abiraterone decanoate) formulation comprising an oil (corn oil, 70%) and benzyl alcohol (10%) and benzyl benzoate (20%) achieved a much higher abiraterone plasma exposure in monkeys when compared with a formulation comprising the same oil vehicle without benzyl benzoate, i.e., corn oil, at 90%, and benzyl alcohol at 10%, which has substantially the same concentration of abiraterone decanoate, and dosed at the same amount.

The solubility of the abiraterone esters can be affected upon adding a co-solvent to the vegetable oil vehicle. In some embodiments, the abiraterone ester is completely dissolved in the composition, and in other embodiments the abiraterone ester is partly dispersed in the composition. In one embodiment, the abiraterone esters are fully dissolved in the vehicle.

The abiraterone prodrug formulations can also contain pharmaceutically acceptable preservatives, polymers, antioxidants, antimicrobials, chelating agents, and other excipients such as citric acid, dextrose, ascorbic acid, benzalkonium chloride, benzoic acid, sodium betadex sulfobutyl ether, calcium chloride, sodium carbomethoxycellulose, chlorobutanol, creatine, croscarmellose, dibasic potassium phosphate, sodium docusate, sodium edetate, glycerin, sodium hyaluronate, hydroxypropyl betadex, lactic acid, lactose, lecithin, maleic acid, mannitol, meglumine, methylcellulose, methylparaben, microcrystalline cellulose, miripitium chloride, momothioglycerol, phenol, poloxamer 188, polyglactin, polysorbate 20, polysorbate 40, polysorbate 80, propylparaben, sodium acetate, sodium benzoate, sodium citrate, sorbitan monolaurate, sorbitol, sucrose, tartaric acid, trisodium citrate, tromantadine, tromethamine, and urea. For example, in some embodiments, the abiraterone prodrug formulations can contain a pharmaceutically acceptable antioxidant, such as monothioglycerol.

The abiraterone prodrug formulations can be sterilized by methods known by persons skilled in the art (for example, gamma irradiation, micron filtration, and autoclaving).

### Long-Acting Release of Abiraterone

The abiraterone prodrugs and abiraterone prodrug formulations (e.g., those containing compounds of Formula I or II as described herein) of the present disclosure are typically formulated to provide a long-acting release of abiraterone to a human subject in need thereof, such as those having a sex hormone-dependent or androgen receptor driven disease or disorder, such as a sex hormone-dependent benign or malignant disorder, an androgen receptor driven cancer, and/or a syndrome due to androgen excess, preferably as a parenteral formulation such as intramuscular, intradermal, or subcutaneous formulation. In some embodiments, the abiraterone prodrugs and abiraterone prodrug formulations (e.g., those containing compounds of Formula I or II as described herein) of the present disclosure can be formulated to deliver therapeutic blood plasma levels of abiraterone over an extended period of time (e.g., at least 1 week, e.g., at least two weeks, at least 3 weeks, at least 4 weeks, and up to six or eight weeks or more, such as up to ten weeks or more, etc.) to subjects having a hormone-dependent benign or malignant disorder, an androgen receptor driven cancer, and/or a syndrome due to androgen excess, following a single administration. In some embodiments, the therapeutic blood plasma concentration of abiraterone can be a concentration of at least 1 ng/ml, e.g., at least 2 ng/ml, at least 4 ng/ml, at least 8 ng/ml. In some embodiments, the therapeutic blood plasma concentration of abiraterone can also be about 0.5 ng/ml or higher. In some embodiments, the therapeutic blood plasma concentration of abiraterone can also be about 0.1 ng/ml or higher.

### Unit Dosage Forms

In some embodiments, the abiraterone prodrugs and abiraterone prodrug formulations (e.g., those containing compounds of Formula I or II as described herein) of the present disclosure can be formulated as a unit dosage form. In some embodiments, the unit dosage form can include a sufficient amount of the respective prodrug such that after a single administration (e.g., intramuscular injection) to a human subject, e.g., a human subject having a sex hormone-dependent or androgen receptor driven disease or disorder, such as a sex hormone-dependent benign or malignant disorder (e.g., metastatic CRPC or metastatic CSPC), an androgen receptor driven cancer, and/or a syndrome due to androgen excess, the unit dosage form provides a therapeutically effective blood plasma concentration of abiraterone in the human subject for a period of at least two weeks, such as at least 3 weeks, at least 4 weeks, at least 5 weeks, and up to six or eight weeks or more, such as up to ten weeks or more, etc. In some embodiments, the therapeutic blood plasma concentration of abiraterone can be a concentration of at least 1 ng/ml, e.g., at least 2 ng/ml, at least 4 ng/ml, at least 8 ng/ml. In some embodiments, the therapeutic blood plasma concentration of abiraterone can also be about 0.5 ng/ml or higher. In some embodiments, the therapeutic blood plasma concentration of abiraterone can also be about 0.1 ng/ml or higher. In some embodiments, the unit dosage form is a parenteral formulation such as intramuscular, intradermal, or subcutaneous formulation. In some embodiments, the unit dosage form is a non-aqueous solution or suspension. In some embodiments, the unit dosage form comprises the abiraterone prodrug (e.g., compound of Formula I or II) dissolved or suspended in a pharmaceutically acceptable oil, e.g., a vegetable oil such as castor oil, corn oil, sesame oil, cottonseed oil, peanut oil, poppy seed oil, tea seed oil, or soybean oil. In some embodiments, two or more different pharmaceutically acceptable oil can be used in the unit dosage forms. In some embodiments, the unit dosage form can further comprise a pharmaceutically acceptable solvent, e.g., an alcohol, an ester, and/or an acid, such as benzyl alcohol, benzyl benzoate, or a combination thereof. Other suitable ingredients for the unit dosage forms include those described herein.

The abiraterone prodrug (e.g., compound of Formula I or II) is typically present in the unit dosage form at a concentration of about 25 mg/ml to about 500 mg/ml (e.g., about 25 mg/ml, about 50 mg/ml, about 100 mg/ml, about 150 mg/ml, about 200 mg/ml, about 250 mg/ml, about 300 mg/ml, about 400 mg/ml, about 500 mg/ml, or any ranges between the recited values). The amount of abiraterone prodrug in the unit dosage forms can vary, depending on various factors such as the clearance rate of the respective abiraterone prodrug, the intended dosing frequency and the desired plasma levels, etc. Typically, the amount of the abiraterone prodrug can be in the range of about 50 mg to about 2000 mg, which if expressed as equivalent of abiraterone, can typically range from about 25 mg to about 1750 mg. In some embodiments, the amount of the abiraterone prodrug can also be higher, such as in the range of about 50 mg to about 5000 mg. In some embodiments, to achieve a less frequent dosing frequency, such as a once a month, once every two months, or once every three months dosing frequency, the prodrug can be included in the unit dosage form at an amount and/or concentration as high as safely tolerable to a human subject user. Typically, the unit dosage form is formulated to have a viscosity suitable for parenteral injection, such as suitable for intramuscular, intradermal, or subcutaneous injection.

In some embodiments, the unit dosage form can be formulated to achieve certain pharmacokinetic (PK) profiles, e.g., a PK profile with a substantially flat curve after an initial rising period. Typically, after the unit dosage form is administered to a human subject, during the initial few hours and up to a few days (e.g., 5 days or a week) post administration, the plasma concentration of abiraterone in the human subject can be increased, which is then gradually plateaued. In some embodiments, after this initial rising period, the plasma concentration of abiraterone in the human subject can be plateaued and can be substantially constant for an extended period of time, for example, for at least a few days (e.g., 2, 3, 4, 5, or 6 days), or for at least 1 week, at least 2 weeks, at least 4 weeks, at least 6 weeks, at least 8 weeks, at least 10 weeks, etc.

In some embodiments, the unit dosage form is suitable for once a month (or once in more than a month, such as once every two months, or once every three months) dosing, and upon a single administration (e.g., intramuscularly) to a human subject in need thereof, the unit dosage form achieves a PK profile characterized by one or more of the following: (a) the unit dosage form provides a therapeutically effective blood plasma concentration of abiraterone in the human subject for at least 4 weeks, such as up to 6 weeks or 8 weeks, or up to 10 weeks or more; (b) a single dose Cₘₐₓ of abiraterone of between about 1 ng/ml and about 300 ng/ml, such as between about 1 ng/ml and about 10 ng/ml; (c) no food effect; (d) a single dose Cₘₐₓ of abiraterone reduced by at least 30% compared to the Cₘₐₓ of abiraterone observed at steady state for a once daily oral dose of Zytiga® at 1000 mg without food; (e) a single dose Cₘᵢₙ of abiraterone at day 28 post administration between about 0.1 ng/ml and about 8 ng/ml; (f) the blood plasma concentration of abiraterone remains substantially constant, e.g., for at least 1 week, e.g., between 1 week and 3 weeks, between 1 week and 10 weeks, or between 2 weeks and 8 weeks post administration. In some embodiments, substantially constant for a period of time can mean that the highest concentration observed for any day (i.e., 24 hours) during that time period is no greater than 4-fold, for example, no greater than 2-fold, of the lowest concentration observed for the same day. No food effect should be generally understood as that no significant differences in PK are observed when the unit dosage form is administered to subjects with food or without food, for example, in some embodiments, no food effect can mean that the Cₘₐₓ and AUC of abiraterone are substantially the same (e.g., between 80% to 125%) between subjects dosed at a fed state or fasted state. A single dose Cₘₐₓ as used herein should be understood as the Cₘₐₓ achieved following a single administration to a treatment naïve subject (generally refers to a human subject who has not received any abiraterone medication within at least 3 days, such as at least 1 week, prior to the administration and with no observable plasma abiraterone prior to the administration). A single dose Cₘᵢₙ used herein refers to the minimum concentration observed for a given day following a single administration to a treatment naïve subject, e.g., at day 28 post administration.

In some embodiments, the unit dosage form is suitable for once a month (or once in more than a month, such as once every two months, or once every three months) dosing, and upon administration (e.g., intramuscularly) of the unit dosage form once in a month (or once in more than a month, such as once every two months, or once every three months) to a human subject in need thereof, the unit dosage form achieves (a) a steady state Cₘₐₓ of abiraterone of between about 1 ng/ml and about 300 ng/ml, such as between about 1 ng/ml and about 10 ng/ml; (b) no food effect: (c) a steady state Cₘₐₓ of abiraterone reduced by at least 30% compared to the Cₘₐₓ of abiraterone observed at steady state for a once daily oral dose of Zytiga® at 1000 mg without food; (d) a steady state Cₘᵢₙ of abiraterone between about 0.1 ng/ml and about 8 ng/ml; and (g) the blood plasma concentration of abiraterone remains substantially constant, e.g., for at least 1 week, e.g., between 1 week and 3 weeks, between 1 week and 10 weeks, or between 2 weeks and 8 weeks post each administration. A steady state Cₘₐₓ or Cₘᵢₙ as used herein should be understood as the Cₘₐₓ or Cₘᵢₙ observed after a steady state is reached, typically following several administrations to a human subject.

In some embodiments, the unit dosage form can be packaged in a container such as a vial or ampule. In some embodiments, the unit dosage form can be included in a pre-filled syringe or in a kit with a syringe, such as a disposable syringe. Other packaging and/or containers are also useful, which are known to those skilled in the art. In some embodiments, a kit comprising multiple unit dosage forms described herein is also provided. In some embodiments, the kit can further comprise a syringe. Typically, one or more (such as 1) unit dosage forms are used to satisfy a desired single dosing amount. In some embodiments, the present disclosure provides abiraterone prodrug formulations that allow multiple single uses. In some embodiments, the present disclosure provides abiraterone prodrug formulations that can be subdivided into multiple unit dosage forms. In some embodiments, the unit dosage form can include any of the pharmaceutical composition described herein, such as those shown in [18]-[30] or [93]-[107] of the Summary Section. In some embodiments, the unit dosage form includes about 180 mg, about 360 mg, about 720 mg, about 1260 mg, about 1800 mg, or any range or value between the recited value, of abiraterone decanoate.

### Exemplary Specific Formulations

In some embodiments, the present disclosure also provides some specific abiraterone prodrug formulations, which can in some embodiments be in a unit dosage form or a multiple unit dosage form. For example, the tables below (Table A and B) show some representative abiraterone ester prodrug formulation in an oil vehicle. All numeric values in the tables should be understood as preceded by the term “about.” The concentration of abiraterone prodrug refers to the amount of abiraterone prodrug in mg per ml of the final formulation, which can be a solution or suspension. The amount of oil (the primary solvent) and co-solvent in the tables is expressed as volume percentage of solvent, which includes both the oil and co-solvent. Suitable oil includes any of the pharmaceutically acceptable oil as described herein. Suitable co-solvents also include any of those described herein, e.g., an alcohol, an ester, and/or an acid, such as benzyl alcohol, benzyl benzoate, or a combination thereof, see e.g., Table B. One example of suitable co-solvents is benzyl alcohol. One example of suitable co-solvents is a combination of benzyl alcohol and benzyl benzoate. In some embodiments, no co-solvent is included in the formulation. In some embodiments, the co-solvent does not include benzyl benzoate. Other optional ingredients are described herein.

TABLE A
Exemplary Formulations

| | Amount/Concentration | | |
|---|---|---|---|
| | | Exemplary | More Exemplary |
| Ingredients | Typical | range | Range |
| Abiraterone prodrug | 25 mg/ml | 50 mg/ml to | 75 mg/ml to 300 |
| (e.g., abiraterone | to 500 mg/ | 300 mg/ml; | mg/ml |
| acetate, abiraterone | ml | 100 mg/ml to | |
| decanoate, abiraterone | | 300 mg/ml | |
| pentanoate, abiraterone | | | |
| hexanoate, abiraterone | | | |
| heptanoate, abiraterone | | | |
| isocaproate, or | | | |
| abiraterone cypionate) | | | |
| Oil (e.g., castor oil, | 50% to | 70% to 100% | 80% to 100% of |
| corn oil) | 100% of | of solvent | solvent, such as |
| | solvent | | 90% |
| Co-solvent (e.g., | 0% to 50% | 0% to 40% or | 0% to 30% or 0% |
| benzyl alcohol, benzyl | of solvent | 0% to 30% of | to 20% of solvent, |
| benzoate, or | | solvent | such as 10%, or |
| combination thereof) | | | 30% |
| Other optional | | | |
| ingredients | | | |

In some embodiments, the present disclosure provides an abiraterone prodrug formulation comprising the abiraterone prodrug and a pharmaceutically acceptable carrier, wherein the pharmaceutically acceptable carrier comprises a pharmaceutically acceptable oil (e.g., described herein), benzyl alcohol, and benzyl benzoate. In some embodiments, the abiraterone prodrug can be abiraterone decanoate. In some embodiments, the abiraterone prodrug can be abiraterone isocaproate. The pharmaceutically acceptable oil typically comprises a triglyceride derived from fatty acids. In some embodiments, the pharmaceutically acceptable oil can be nature oil, synthetic oil, or semi-synthetic oil, such as fractionated coconut oil and medium-chain triglycerides, such as those sold under the trademark Miglyol. In some embodiments, the pharmaceutically acceptable oil can be selected from vegetable oil, castor oil, corn oil, sesame oil, cottonseed oil, peanut oil (arachis oil), poppy seed oil, tea seed oil, and soybean oil. In some embodiments, the present disclosure provides certain exemplary formulations shown in Table B.

TABLE B
Further Exemplary Formulations

| | Amount/Concentration | | |
|---|---|---|---|
| | | | More Exemplary |
| Ingredients | Typical | Exemplary range | Range |
| Abiraterone prodrug | 25 mg/ml to 500 | 50 mg/ml to 300 | 75 mg/ml to 300 |
| (e.g., abiraterone | mg/ml | mg/ml; 100 mg/ml to | mg/ml, such as 150 |
| decanoate or | | 300 mg/ml | mg/ml to about 250 |
| abiraterone | | | mg/ml |
| isocaproate) | | | |
| Oil (e.g., corn oil, | 30% to 100% of | 50% to 90% of solvent | 60% to 90% of |
| sesame oil, peanut oil, | solvent | | solvent, such as 70% |
| cottonseed oil, and/or | | | |
| Miglyol 812) | | | |
| Co-solvent 1 (e.g., | 0% to 20% of | 0% to 15% of solvent | 0% to 10% of solvent, |
| benzyl alcohol) | solvent | | such as 10% |
| benzyl benzoate | 0% to 50% of | 0% to 35% of solvent | 0% to 30% of solvent, |
| | solvent | | such as 20% |
| Other optional | | | |
| ingredients | | | |

As used herein, when the solvent system of an abiraterone prodrug formulation comprises two or more solvents (including oil), the abiraterone prodrug formulation may be expressed as an abiraterone prodrug solution in the solvent system having x % of an oil and y % of a co-solvent (e.g., 90% corn oil and 10% benzyl alcohol) at a specified concentration. In such expressions, whether or not followed by “v/v,” the x % and y % should be understood as based on volume percentages, unless otherwise specified or obviously contrary from context.

### Combination Treatment

In some embodiments, the method herein can comprise administering one or more other drug or agent (for example, another cancer chemotherapeutic drug, hormone replacement drug, or hormone ablation drug) to the human subject, either concurrently or sequentially with the administration of the abiraterone prodrug, through the same route or a different route of administration. In some embodiments, the other drug or agent can be a steroid, such as prednisone, prednisolone, and/or methylprednisolone. In some embodiments, the other drug or agent can be a chemotherapy drug, such as paclitaxel, mitoxantrone, and/or docetaxel. In some embodiments of the method herein, the other agent or drug can be a GnRH agonist, such as Leuprolide, deslorelin, goserelin, or triptorelin, e.g., leuprolide acetate (e.g., a long-acting IM injectable formulation). In some embodiments, the other agent or drug can be seocalcitol, bicalutamide, flutamide, a glucocorticoid including, but not limited to, hydrocortisone, prednisone, prednisolone, or dexamethasone. The amount of the other drugs or agents to be administered can vary, typically can be an amount that is effective in treating the respective disease or disorder (e.g., prostate cancer) either alone or in combination with the abiraterone prodrug or abiraterone prodrug formulation of the present disclosure.

Additional suitable other drugs or agents include those described herein. For example, useful other drugs or agents include, but are not limited to, anticancer agents, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, and anti-androgens.

For example, suitable anti-cancer agents, including but not limited to, acemannan, aclarubicin, aldesleukin, alemtuzumab, alitretinoin, altretamine, amifostine, amsacrine, anagrelide, anastrozole, ancestim, bexarotene, broxuridine, capecitabine, celmoleukin, cetrorelix, cladribine, clotrimazole, daclizumab, dexrazoxane, dilazep, docosanol, doxifluridine, bromocriptine, carmustine, cytarabine, diclofenac, edelfosine, edrecolomab, eflornithine, emitefur, exemestane, exisulind, fadrozole, filgrastim, finasteride, fludarabine phosphate, formestane, fotemustine, gallium nitrate, gemcitabine, glycopine, heptaplatin, ibandronic acid, imiquimod, iobenguane, irinotecan, irsogladine, lanreotide, leflunomide, lenograstim, lentinan sulfate, letrozole, liarozole, lobaplatin, lonidamine, masoprocol, melarsoprol, metoclopramide, mifepristone, miltefosine, mirimostim, mitoguazone, mitolactol, molgramostim, nafarelin, nartograstim, nedaplatin, nilutamide, noscapine, oprelvekin, osaterone, oxaliplatin, pamidronic acid, pegaspargase, pentosan polysulfate sodium, pentostatin, picibanil, pirarubicin, porfimer sodium, raloxifene, raltitrexed, rasburicase, rituximab, romurtide, sargramostim, sizofiran, sobuzoxane, sonermin, suramin, tasonermin, tazarotene, tegafur, temoporfin, temozolomide, teniposide, tetrachlorodecaoxide, thalidomide, thymalfasin, thyrotropin alfa, topotecan, toremifene, trastuzumab, treosulfan, tretinoin, trilostane, trimetrexate, ubenimex, valrubicin, verteporfin, vinorelbine. Suitable anti-androgen agents include but are not limited to bicalutamide, flutamide and nilutamide. Suitable differentiating agents include, but are not limited to, polyamine inhibitors; vitamin D and its analogs, such as, calcitriol, doxercalciferol and seocalcitol; metabolites of vitamin A, such as, ATRA, retinoic acid, retinoids; short-chain fatty acids; phenylbutyrate; and nonsteroidal anti-inflammatory agents, anti-neoplastic agent, including, but not limited to, tubulin interacting agents, topoisomerase inhibitors and agents, acitretin, alstonine, amonafide, amphethinile, amsacrine, ankinomycin, anti-neoplaston, aphidicolin glycinate, asparaginase, baccharin, batracylin, benfluron, benzotript, bromofosfamide, caracemide, carmethizole hydrochloride, chlorsulfaquinoxalone, clanfenur, claviridenone, crisnatol, curaderm, cytarabine, cytocytin, dacarbazine, datelliptinium, dihaematoporphyrin ether, dihydrolenperone, dinaline, distamycin, docetaxel, elliprabin, elliptinium acetate, epothilones, ergotamine, etoposide, etretinate, fenretinide, gallium nitrate, genkwadaphnin, hexadecylphosphocholine, homoharringtonine, hydroxyurea, ilmofosine, isoglutamine, isotretinoin, leukoregulin, lonidamine, merbarone, merocyanlne derivatives, methylanilinoacridine, minactivin, mitonafide, mitoquidone, mitoxantrone, mopidamol, motretinide, N-(retinoyl)amino acids, N-acylated-dehydroalanines, nafazatrom, nocodazole derivative, ocreotide, oquizanocine, paclitaxel, pancratistatin, pazelliptine, piroxantrone, polyhaematoporphyrin, polypreic acid, probimane, procarbazine, proglumide, razoxane, retelliptine, spatol, spirocyclopropane derivatives, spirogermanium, strypoldinone, superoxide dismutase, teniposide, thaliblastine, tocotrienol, topotecan, ukrain, vinblastine sulfate, vincristine, vindesine, vinestramide, vinorelbine, vintriptol, vinzolidine, and withanolides, a kinase inhibitor including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, SOD mimics or αᵥβ₃ inhibitors. Suitable anti-metabolite agents may be selected from, but not limited to, 5-FU-fibrinogen, acanthifolic acid, aminothiadiazole, brequinar sodium, carmofur, cyclopentyl cytosine, cytarabine phosphate stearate, cytarabine conjugates, dezaguanine, dideoxycytidine, dideoxyguanosine, didox, doxifluridine, fazarabine, floxuridine, fludarabine phosphate, 5-fluorouracil, N-(2′-furanidyl)-5-fluorouracil, isopropyl pyrrolizine, methobenzaprim, methotrexate, norspermidine, pentostatin, piritrexim, plicamycin, thioguanine, tiazofurin, trimetrexate, tyrosine kinase inhibitors, and uricytin. Suitable alkylating agents may be selected from, but not limited to, aldo-phosphamide analogues, altretamine, anaxirone, bestrabucil, budotitane, carboplatin, carmustine, chlorambucil, cisplatin, cyclophosphamide, cyplatate, diphenylspiromustine, diplatinum cytostatic, elmustine, estramustine phosphate sodium, fotemustine, hepsul-fam, ifosfamide, iproplatin, lomustine, mafosfamide, mitolactol, oxaliplatin, prednimustine, ranimustine, semustine, spiromustine, tauromustine, temozolomide, teroxirone, tetraplatin and trimelamol. Suitable antibiotic agents may be selected from, but not limited to, aclarubicin, actinomycin D, actinoplanone, adriamycin, aeroplysinin derivative, amrubicin, anthracycline, azino-mycin-A, bisucaberin, bleomycin sulfate, bryostatin-1, calichemycin, chromoximycin, dactinomycin, daunorubicin, ditrisarubicin B, dexamethasone, doxorubicin, doxorubicin-fibrinogen, elsamicin-A, epirubicin, erbstatin, esorubicin, esperamicin-A1, esperamicin-Alb, fostriecin, glidobactin, gregatin-A, grincamycin, herbimycin, corticosteroids such as hydrocortisone, idarubicin, illudins, kazusamycin, kesarirhodins, menogaril, mitomycin, neoenactin, oxalysine, oxaunomycin, peplomycin, pilatin, pirarubicin, porothramycin, prednisone, prednisolone, pyrindanycin A, rapamycin, rhizoxin, rodorubicin, sibanomicin, siwenmycin, sorangicin-A, sparsomycin, talisomycin, terpentecin, thrazine, tricrozarin A, and zorubicin. Non-limiting examples of suitable steroids include hydrocortisone, prednisone, prednisolone, or dexamethasone.

### Combination Treatment for Prostate Cancer

Prostate cancer treatments often involve multiple therapies, including for example, radiotherapy, surgery, androgen deprivation therapy, hormone therapy, chemotherapy, immunotherapy, and various drug combinations. A search in the website clinicaltrials.gov identified more than 250 clinical trials with abiraterone/abiraterone acetate listed as an intervention agent, and many of such clinical trials include a combination therapy for treating prostate cancer. As discussed herein, compared to oral abiraterone acetate formulation, the abiraterone prodrugs herein can provide increased bioavailability, elimination of the food effect, reduced pill burden, less frequent dosing frequency, and sustained effective blood plasma levels of abiraterone, and prolonged CYP17A1 inhibition, with reduction of dihydrotestosterone, and testosterone levels up to 70 days or more following administration of the abiraterone prodrug formulation without the undesired increase of steroids upstream of CYP17A1 hydroxylase such as progesterone. Considering their superior pharmacokinetic and/or pharmacodynamics profiles, the abiraterone prodrugs herein can also be advantageously used in various combination therapies to replace or supplement the oral administration of abiraterone acetate.

In some embodiments, the present disclosure provides a method of treating prostate cancer (e.g., any of those described herein) in a human subject in need thereof with a combination therapy, which comprises administering to the human subject a therapeutically effective amount of the abiraterone prodrug (e.g., abiraterone decanoate) or the abiraterone prodrug formulation herein, and one or more additional therapies. The one or more additional therapies can be administered to the human subject concurrently or sequentially in any order with administering the abiraterone prodrug or abiraterone prodrug formulation herein, which can be via the same or different route of administration. In some embodiments, the method herein comprises treating the human subject with a radiotherapy or surgery. In some embodiments, the method comprises administering to the human subject one or more other agents selected from anticancer agents, hormone ablation agents, anti-androgen agents, differentiating agents, anti-neoplastic agents, kinase inhibitors, anti-metabolite agents, alkylating agents, antibiotic agents, immunological agents, interferon-type agents, intercalating agents, growth factor inhibitors, cell cycle inhibitors, enzymes, topoisomerase inhibitors, biological response modifiers, mitotic inhibitors, matrix metalloprotease inhibitors, genetic therapeutics, or combinations thereof. In some embodiments, the method comprises administering to the human subject one or more other agents selected from a chemotherapeutic drug, hormone replacement drug, or hormone ablation drug. In some embodiments, the method comprises treating the human subject with an androgen deprivation therapy. While many of the combination therapies below are described as in connection with various treatments for prostate cancer, the present disclosure is not so limited. And in some embodiments, the combination therapies described below can also be used in the treatment of other diseases or disorders described herein, such as other cancers described herein.

In more particular embodiments, the combination therapy typically includes administering to the human subject a glucocorticoid. For example, in some embodiments, the method comprises administering to the human subject one or more agents selected from hydrocortisone, prednisone, prednisolone, methylprednisolone, and dexamethasone, preferably, dexamethasone. However, in some embodiments, a glucocorticoid replacement therapy (e.g., administering a glucocorticoid, such as hydrocortisone, prednisone, prednisolone, methylprednisolone, or dexamethasone) is not desired. For example, a glucocorticoid may be contraindicated for the human subject, who may have an underlying condition, such as diabetics. In some embodiments, the method can also be characterized in that the human subject is not treated with a glucocorticoid replacement therapy. In some embodiments, the human subject is not treated with an agent selected from hydrocortisone, prednisone, prednisolone, methylprednisolone, and dexamethasone. In some embodiments, the method can comprise administering to the human subject a mineralocorticoid receptor antagonist, such as eplerenone. For example, in any of the embodiments herein when glucocorticoid replacement therapy is not desired and/or not administered, the method can comprise administering to the human subject a mineralocorticoid receptor antagonist, such as eplerenone. In some embodiments, an agent for treating a mineralocorticoid toxicity is not needed and not administered to the human subject. In some embodiments, the method can also be characterized in that the human subject is not treated with an agent effective for treating a mineralocorticoid toxicity. In some embodiments, the method can be characterized in that the human subject is not treated with an agent that is a glucocorticoid or a mineralocorticoid receptor antagonist.

The combination therapy for the method herein can also include an androgen deprivation therapy, such as through administering to the human subject a gonadotropin-releasing hormone (GnRH) analog. When included, suitable GnRH analogs for the combination therapy are not particularly limited and include both GnRH agonists and GnRH antagonists. For example, in some embodiments, the method can comprise administering to the human subject a gonadotropin-releasing hormone (GnRH) agonist, such as buserelin, leuprolide, deslorelin, fertirelin, histrelin, gonadorelin, lecirelin, goserelin, nafarelin, peforelin or triptorelin, and/or a GnRH antagonist, such as abarelix, cetrorelix, degarelix, ganirelix, elagolix, linzagolixa, or relugolix. In some embodiments, the human subject is not administered any of the GnRH agonists and GnRH antagonists described herein.

### Inhibition of Androgen Receptor Activities

In some embodiments, the combination therapy includes treating the human subject to reduce androgen receptor (AR) activities, such as an AR antagonist or an agent otherwise downregulating or inhibiting AR activities.

In some embodiments, the method can include administering to the human subject an androgen receptor (AR) antagonist. Various AR antagonists are known in the art, which include without limitation 1^{st} and 2^{nd}-generations AR antagonists, see e.g., Rice, M. A., et al. Front Oncol. 9:801 (2019), and third-generation AR antagonists, such as an N-terminal domain inhibitor. In some embodiments, the method comprises administering to the human subject a 1^{st}-generation androgen receptor antagonist, which includes without limitation, proxalutamide, bicalutamide, flutamide, nilutamide, topilutamide, etc. In some embodiments, the method comprises administering to the human subject a 2^{nd}-generation androgen receptor antagonist, which includes without limitation, for example, apalutamide, darolutamide or enzalutamide. In some embodiments, the method comprises administering to the human subject apalutamide. In some preferred embodiments, the method comprises administering to the human subject enzalutamide. In some embodiments, the method comprises administering to the human subject a 3^{rd}-generation androgen receptor antagonist, such as an N-terminal domain inhibitor. N-terminal domain inhibitors are known in the art. Non-limiting useful examples include any of those described in U.S. Application Publication No. 2020/0123117, the content of which is herein incorporated by reference. It should be noted that in embodiments where an AR antagonist is administered, one or more such antagonists can be administered, which can be selected from 1^{st}, 2^{nd} or 3^{rd} AR antagonists alone, or in any combination.

In addition to agents directly targeting androgen receptor, other methods and/or agents that modulate androgen receptor activities, including for example, modulation of upstream kinase activities and/or androgen receptor transcriptional activities, can also be used in the combination therapy herein. For example, in some embodiments, the combination therapy can include administering to the human subject one or more upstream kinase modulators, the activation or inhibition of which can reduce AR activities. Such upstream kinases are known in the art, for example, as described in Shah, K. and Bradbury, N. A., Cancer cell microenviron. 2(4):doi:10.14800/ccm.1023 (2015), and Koul H. K. et al. Genes & Cancer 4(9-10):342-359 (2013). In some embodiments, the method comprises administering to the human subject one or more kinase modulators selected from FLT-3 (FMS-like tyrosine kinase) inhibitors, AXL (anexclckto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof. Suitable kinase modulators/inhibitors are not particularly limited, which include any of those known, for example, small molecule drugs, polypeptides including antibodies such as monoclonal antibodies or antigen binding fragments thereof, RNA or DNA based agents.

In some embodiments, the combination therapy can include administering to the human subject an agent that downregulates AR or otherwise inhibits AR activities. Without wishing to be bound by theories, AR activities can be affected on the genomic and/or the transcription level of AR itself, or the genomic and/or the transcription level of those upstream targets of AR that play a role in regulating AR activities and those downstream targets that are regulated by AR, using a variety of molecules which interfere with transcription and/or translation (e.g., RNA silencing agents (e.g., antisense, siRNA, shRNA, micro-RNA), Ribozyme and DNAzyme), or on the protein level using e.g., antagonists, enzymes that cleave the polypeptide, small molecules that interfere with the protein's activity (e.g., competitive ligands) and the like.

In some embodiments, downregulation of AR or inhibition of AR activities can be achieved through RNA silencing of a target gene (e.g., AR or suitable upstream and downstream targets of AR as described herein, etc.). As used herein, the phrase “RNA silencing” refers to a group of regulatory mechanisms (e.g., RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In some embodiments, the RNA silencing agent is capable of preventing complete processing (e.g., the full translation and/or expression) of an mRNA molecule through a post-transcriptional silencing mechanism. RNA silencing agents include noncoding RNA molecules, for example, RNA duplexes comprising paired strands, as well as precursor RNAs from which such small non-coding RNAs can be generated. Exemplary RNA silencing agents include double-stranded RNAs (dsRNAs) such as short interfering RNAs (siRNAs), miRNAs and shRNAs. In one embodiment, the RNA silencing agent is capable of inducing RNA interference. In another embodiment, the RNA silencing agent is capable of mediating translational repression. The strands of a double-stranded interfering RNA (e.g., an siRNA) may be connected to form a hairpin or stem-loop structure (e.g., an shRNA or sh-RNA). Thus, as mentioned, the RNA silencing agent of some embodiments of the disclosure may also be a short hairpin RNA (shRNA).

It will be appreciated that the RNA silencing agent of some embodiments of the present disclosure need not be limited to those molecules containing only RNA, but further encompasses chemically modified nucleotides and non-nucleotides.

In some embodiments, the RNA silencing agent provided herein can be functionally associated with a cell-penetrating peptide. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell.

According to another embodiment, the RNA silencing agent may be a miRNA or a mimic thereof. The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology. The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs).

Downregulation of AR or inhibition of AR activities can also be achieved by gene editing of a target gene (e.g., AR or suitable upstream and downstream targets of AR as described herein, etc.). Gene editing can be performed, for example, with a clustered regularly interspaced short palindromic repeats CRISPR-CAS9 system. CRISPR-CAS9 systems have been described in the literature and can include, for example, CAS9 and a guide RNA. Other gene editing techniques have also been described in the literature and can also be used.

Another agent capable of downregulating a target (e.g., AR or suitable upstream and downstream targets of AR as described herein, etc.) is a DNAzyme molecule capable of specifically cleaving an mRNA transcript or DNA sequence of the target. DNAzymes are single-stranded polynucleotides which are capable of cleaving both single and double stranded target sequences. (Breaker et al., Chemistry and Biology 1995; 2:655; Santoro et al., Proc. Natl. Acad. Sci. USA 1997; 943:4262.) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions. (Santoro et al., Khachigian, Curr. Opin. Mol. Ther. 2002; 4:119-121.)

Downregulation of a target (e.g., AR or suitable upstream and downstream targets of AR as described herein, etc.) can also be affected by using an antisense polynucleotide capable of specifically hybridizing with an mRNA transcript encoding the target.

Another agent capable of downregulating a target (e.g., AR or suitable upstream and downstream targets of AR as described herein, etc.) is a ribozyme molecule capable of specifically cleaving an mRNA transcript encoding a target. Ribozymes are being increasingly used for the sequence-specific inhibition of gene expression by the cleavage of mRNAs encoding proteins of interest. (Welch et al., Curr. Opin. Biotechnol. 1998; 9:486-96.)

Another agent capable of downregulating a target (e.g., AR or suitable upstream and downstream targets of AR as described herein, etc.) is any molecule which binds to and/or cleaves the target. Such molecules can be antagonists of the target, or inhibitory peptides of the target.

Another agent which can be used along with some embodiments of the present disclosure to downregulate a target (e.g., AR or suitable upstream and downstream targets of AR as described herein, etc.) is a molecule which prevents target activation and/or substrate binding.

Another agent which can be used along with some embodiments of the present disclosure to downregulate AR or inhibit AR's activities is an androgen receptor degrader, such as those based on PROteolysis TArgeting Chimeric (PROTAC) technology. See, e.g., Kregel, S. et al. Neoplasia 22(2):111-119 (2020).

Another agent which can be used along with some embodiments of the present disclosure to downregulate a target (e.g., AR or suitable upstream and downstream targets of AR as described herein, etc.) is to repress or downregulate the activation of the target's transcriptional activity, more particularly, AR's transcriptional activities. For example, such agent can interfere with the nuclear translocation of AR, downregulate the protein level of AR, decrease hormone binding to AR, interfere with recruitment of transcriptional cofactors (e.g., steroid receptor coactivator 1 (SRC1) and transcriptional intermediary factor 2 (TIF2)), interfere with AR-DNA-binding, e.g., the binding to specific DNA response elements (AREs or, androgen response elements), inhibit AR recruitment to an AR target gene enhancer, and/or inhibit AR-chromatin binding etc. or otherwise inhibit the DNA-binding-dependent or non-DNA-binding-dependent AR signaling pathways. Suitable agents that can inhibit or interfere with AR transcriptional activities include any of those known in the art and any of those agents exemplified herein that are capable of inhibiting or interfering with such activities. For example, certain AR antagonists such as the 1^{st} generation AR antagonists (e.g., bicalutamide) are known to inhibit AR transcriptional activities by inhibiting nuclear translocation of AR. Other agents, such as arsenic compounds (e.g., arsenic trioxide), were also known to inhibit AR transcriptional activity. See e.g., Rosenblatt A. E., et al, Mol. Endocrinol. 23(3):412-421 (2009).

In some embodiments, the combination therapy can include administering to the human subject one or more chemotherapeutic agents. Suitable chemotherapeutic agents include any of those known in the art. In some embodiments, the method comprises administering to the human subject a taxane based chemotherapeutic agent (e.g., docetaxel, cabazitaxel, paclitaxel, etc.) and/or platinum based chemotherapeutic agent (e.g., cisplatin, carboplatin, oxaliplatin, etc.).

In some embodiments, the combination therapy can include treating the human subject with a radiotherapy. Suitable radiotherapy includes any of those known in the art. In some embodiments, the method comprises treating the human subject with stereotactic body radiotherapy or neutron radiation.

In some embodiments, the combination therapy can include treating the human subject with Radium-223, e.g., Xofigo (Radium-223 dichloride) injection.

In some embodiments, the combination therapy can include administering to the human subject one or more immunotherapies. Suitable immunotherapies include any of those known in the art. In some embodiments, the method comprises administering to the human subject Sipuleucel-T. In some embodiments, the method comprises administering to the human subject an immune checkpoint inhibitor. For example, in some embodiments, the method comprises administering to the human subject an anti-PD-1 antibody, such as pembrolizumab or nivolumab, and/or an anti-PD-L1 antibody, such as avelumab or atezolizumab. In some embodiments, the method comprises administering to the human subject an anti-CTLA-4 antibody, such as ipilimumab.

In some embodiments, the combination therapy can include administering to the human subject a bispecific T-cell engager (BiTE) therapy, such as blinatumomab or solitomab.

In some embodiments, the combination therapy can include administering to the human subject one or more poly ADP ribose polymerase (PARP) inhibitors. In some embodiments, the human subject having prostate cancer also has DNA repair defects. In some embodiments, the human subject having prostate cancer does not have DNA repair defects. Suitable PARP inhibitors include any of those known in the art. For example, in some embodiments, the method comprises administering to the human subject a PARP inhibitor selected from niraparib, rucaparib, olaparib, talazoparib, veliparib, and fluzoparib.

In some embodiments, the combination therapy can include administering to the human subject one or more kinase inhibitors. In some embodiments, the human subject is characterized as having an abnormal level of the respective kinase. In some embodiments, the kinase inhibitor can reduce the activity of androgen receptor or otherwise beneficial to cancer treatment. Suitable kinase inhibitors include any of those known in the art. For example, in some embodiments, the method comprises administering to the human subject a kinase inhibitor selected from sunitinib, dasatinib, cabozantinib, erdafitinib, dovitinib, capivasertib, onvansertib, ipatasertib, afuresertib, alisertib, apitolisib, and opaganib.

In some embodiments, the combination therapy can include administering to the human subject one or more bone protecting agents. In such embodiments, typically, the human subject is characterized as having prostate cancer (e.g., CRPC) with bone metastasis. Suitable bone protecting agents include any of those known in the art. For example, in some embodiments, the method comprises administering to the human subject a bone protecting agent selected from denosumab and zolendronic acid.

In some embodiments, the combination therapy can include administering to the human subject one or more additional agents that are useful for treating prostate cancer, by itself or in combination with an abiraterone medication such as the abiraterone prodrugs herein. Such additional agents are not particularly limited. For example, in some embodiments, the method comprises administering to the human subject a therapeutic agent selected from 1) an anti-IL23 targeting monoclonal antibody, e.g., tildrakizumab; 2) a selenium, such as sodium selenite; 3) an EZH2 inhibitor, e.g., CPI-1205, GSK2816126, or tazemetostat; 4) a CDK4/6 inhibitor, e.g., palbociclib, ribociclib, abemaciclib; 6) a bromodomain and extra-terminal domain (BET) inhibitor, e.g., CCS1477, INCB057643, alobresib, ZEN-3694, or molibresib (GSK525762); 7) an anti-CD105 antibody, e.g., TRC105 or carotuximab; 8) niclosamide; 9) an A2A receptor antagonist, e.g., AZD4635; 10) a phosphoinositide 3-kinase (PI3K) inhibitor, e.g., AZD-8186, buparlisib, or dactolisib; 11) a further non-steroidal CYP17A1 inhibitor, e.g. seviteronel; 12) an antiprogestogen, e.g., onapristone; 13) navitoclax; 14) an HSP90 inhibitor, e.g., onalespib (AT13387); 15) an HSP27 inhibitor, e.g., OGX-427; 16) a 5-alpha-reductase inhibitor, e.g., dutasteride; 17) metformin; 18) AMG-386; 19) dextromethorphan; 20) theophylline; 21) hydroxychloroquine; and 22) lenalidomide. In some embodiments, the combination therapy can include administering to the human subject one or more one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof. In some embodiments, a cell therapy, such as a T cell mediated cell therapy including central memory T cells, can also be part of the combination therapy.

In some embodiments, the combination therapy can include administering to the human subject one or more agents selected from 1) a poly (ADP-ribose) polymerase (PARP) inhibitor including but not limited to olaparib, niraparib, rucaparib, talazoparib; 2) an androgen receptor ligand binding domain inhibitor including but not limited to enzalutamide, apalutamide, darolutamide, bicalutamide, nilutamide, flutamide, ODM-204, TAS3681; 3) an additional inhibitor of CYP17A1 including but not limited to galeterone, abiraterone, abiraterone acetate; 4) a microtubule inhibitor including but not limited to docetaxel, paclitaxel, cabazitaxel (XRP-6258); 5) a modulator of PD-1 or PD-L1 including but not limited to pembrolizumab, durvalumab, nivolumab, atezolizumab; 6) a gonadotropin releasing hormone agonist including but not limited to cyproterone acetate, leuprolide; 7) a 5-alpha reductase inhibitor including but not limited to finasteride, dutasteride, turosteride, bexlosteride, izonsteride, FCE 28260, SKF105,111; 8) a vascular endothelial growth factor inhibitor including but not limited to bevacizumab (Avastin); 9) a histone deacetylase inhibitor including but not limited to OSU-HDAC42; 10) an integrin alpha-v-beta-3 inhibitor including but not limited to VITAXIN; 11) a receptor tyrosine kinase inhibitor including but not limited to sunitumib; 12) a phosphoinositide 3-kinase inhibitor including but not limited to alpelisib, buparlisib, idealisib; 13) an anaplastic lymphoma kinase (ALK) inhibitor including but not limited to crizotinib, alectinib; 14) an endothelin receptor A antagonist including but not limited to ZD-4054; 15) an anti-CTLA4 inhibitor including but not limited to MDX-010 (ipilimumab); 16) an heat shock protein 27 (HSP27) inhibitor including but not limited to OGX 427; 17) an androgen receptor degrader including but not limited to ARV-330, ARV-110; 18) an androgen receptor DNA-binding domain inhibitor including but not limited to VPC-14449; 19) a bromodomain and extra-terminal motif (BET) inhibitor including but not limited to BI-894999, GSK525762, GS-5829; 20) an androgen receptor N-terminal domain inhibitor including but not limited to a sintokamide; 21) an alpha-particle emitting radioactive therapeutic agent including but not limited to radium 233 or a salt thereof; 22) niclosamide; or related compounds thereof; 23) a selective estrogen receptor modulator (SERM) including but not limited to tamoxifen, raloxifene, toremifene, arzoxifene, bazedoxifene, pipindoxifene, lasofoxifene, enclomiphene; 24) a selective estrogen receptor degrader (SERD) including but not limited to fulvestrant, ZB716, OP-1074, elacestrant, AZD9496, GDC0810, GDC0927, GW5638, GW7604; 25) an aromitase inhibitor including but not limited to anastrazole, exemestane, letrozole; 26) selective progesterone receptor modulators (SPRM) including but not limited to mifepristone, lonaprison, onapristone, asoprisnil, lonaprisnil, ulipristal, telapristone; 27) a glucocorticoid receptor inhibitor including but not limited to mifepristone, COR108297, COR125281, ORIC-101, PT150; 28) CDK4/6 inhibitors including palbociclib, abemaciclib, ribociclib; 29) HER2 receptor antagonist including but not limited to trastuzumab, neratinib; and 30) a mammalian target of rapamycin (mTOR) inhibitor including but not limited to everolimus, temsirolimus.

The combination therapy herein is not particularly limited to any specific numbers of additional therapies. For example, in addition to administering the abiraterone prodrug or abiraterone prodrug formulation herein and an optional glucocorticoid such as hydrocortisone, prednisone, prednisolone, methylprednisolone, and dexamethasone, the combination therapy typically can include additional 1, 2, 3, 4, 5, 6, or more therapies described herein. For example, in some embodiments, the combination therapy can include one additional therapy, e.g., any one of those described herein, for example, a GnRH agonist, a GnRH antagonist, an androgen receptor antagonist, a chemotherapy, a PARP inhibitor, a kinase inhibitor, an immunotherapy, a radiation therapy, surgery, an androgen deprivation therapy, etc. In some embodiments, the combination therapy can include two or more additional therapies described herein. For example, in some particular embodiments, the combination therapy can include administering to the human subject a PARP inhibitor and an androgen deprivation therapy. In some embodiments, the combination therapy can include administering to the human subject a GnRH agonist and a radiation therapy. In some embodiments, the combination therapy can include administering to the human subject a GnRH agonist, a chemotherapeutic agent, and a radiation therapy. In some embodiments, the combination therapy can include administering to the human subject an androgen receptor antagonist (e.g., 1^{st}, 2^{nd} and/or 3^{rd} generation AR antagonist), a GnRH agonist, and optionally a radiation therapy, a chemotherapeutic agent, indomethacin, or 5-alpha reductase inhibitor. In some embodiments, the combination therapy can include administering to the human subject an androgen receptor antagonist (e.g., 1^{st}, 2^{nd} and/or 3^{rd} generation AR antagonist) and a radiation therapy. In some embodiments, the combination therapy can include administering to the human subject an androgen receptor antagonist (e.g., 1^{st}, 2^{nd}, and/or 3^{rd} generation AR antagonist) and a chemotherapeutic agent. In some embodiments, the combination therapy can include administering to the human subject an androgen receptor antagonist (e.g., 1^{st}, 2^{nd} and/or 3^{rd} generation AR antagonist) and an anti-CTLA4 antibody. It should be understood that these combinations discussed are examples of useful combinations, which are in no way limiting, and other combinations of the additional therapies described herein are allowed. However, in some embodiments, the combination therapy does not include administering to the human subject a GnRH agonist, a GnRH antagonist, an androgen deprivation therapy, and/or does not include castration of the human subject.

In any of the combination therapies described herein, unless otherwise specified or contrary from context, the method can comprise administering abiraterone decanoate as described herein or the pharmaceutical composition comprising the abiraterone decanoate as described herein, in combination with the one or more additional therapies.

It should be noted that in some embodiments, the method of treating prostate cancer (e.g., any of those described herein) herein is not in conjunction with a combination therapy. For example, the method comprises administering to the human subject a therapeutically effective amount of the abiraterone prodrug (e.g., abiraterone decanoate) or the abiraterone prodrug formulation herein, without the one or more additional therapies described herein.

### Dosing Regimen

The abiraterone prodrugs and formulations of the present disclosure can generally provide a long-acting release of abiraterone to a human subject user. This long-acting release profile allows administering abiraterone to a human subject user at a low dosing frequency, such as once a week, once a month, once every two months, once every three months, or even less frequently, which can improve patient compliance and reduce pill burdens.

In some embodiments, the method herein can have a dosing regimen of once a week or once in more than a week. Typically, the dosing frequency can range from once a week to once every few months, such as from once a week to once every eight weeks, or from once a week to once every three months, e.g., once a month, once every two months, or once every three months. In some embodiments, the dosing amount for each dose is about 50 mg to about 5000 mg (e.g., about 500 mg, about 1000 mg, about 1500 mg, about 2000 mg, about 5000 mg, or any ranges between the recited values) of abiraterone prodrug. In some embodiments, the dosing amount of abiraterone prodrug for each dose is about 0.5 mg/kg to about 200 mg/kg (e.g., about 0.5 mg/kg, about 1 mg/kg, about 5 mg/kg, about 10 mg/kg, about 20 mg/kg, about 30 mg/kg, about 50 mg/kg, about 90 mg/kg, about 100 mg/kg, about 200 mg/kg, or any ranges between the recited values) of body weight of a human subject.

Abiraterone prodrugs suitable for use for a once a week or once in more than a week dosing methods above include those described herein. In some embodiments, the abiraterone prodrug can be a lipophilic ester of abiraterone described herein, for example, an acetate, a propionate, a butanoate, a (vaterate) pentanoate, an isocaproate, a buciclate, a cyclohexanecarboxylate, a phenyl propionate, caproate (hexanoate), a enanthate (heptanoate), a cypionate, an octanoate, a noncanoate, a decanoate, an undecanoate, a dodecanoate, a tridecanoate, a tetradecanoate, a pentadecanoates, and a hexadecanoate. In some preferred embodiments, the abiraterone prodrug can be a compound of Formula I, for example, a compound of Formula I, wherein R¹ is a C₇₋₁₆ alkyl, e.g., an alkyl having a formula of —(CH₂)ₙ—CH₃, wherein n is an integer between 6 and 12 (e.g., n is 6, 7, 8, 9, 10); or R¹ is represented by the formula —(CH₂)ₙ-Cy, wherein n is an integer of 1-6, and Cy is a C₃₋₆ cycloalkyl or phenyl, for example, in more specific embodiments, n can be 1 or 2, and Cy is cyclopentyl, cyclohexyl, or phenyl; or R¹ is

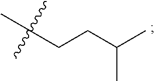 [SMILES: *CCC(C)C]

### or R¹ is

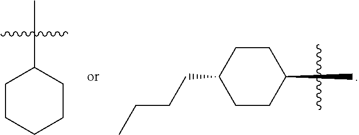

In some embodiments, the abiraterone prodrug can be a compound of Formula II, wherein R² in Formula II can be a C₁₋₁₆ alkyl, e.g., an alkyl having a formula of —(CH₂)ₙ—CH₃, wherein n is an integer between 0 and 12; or R² in Formula TT can be represented by the formula —(CH₂)ₙ-Cy, wherein n is an integer of 1-6, and Cy is a C₃₋₆ cycloalkyl or phenyl, for example, in more specific embodiments, n can be 1 or 2, and Cy is cyclopentyl, cyclohexyl, or phenyl; or R² in Formula II can be

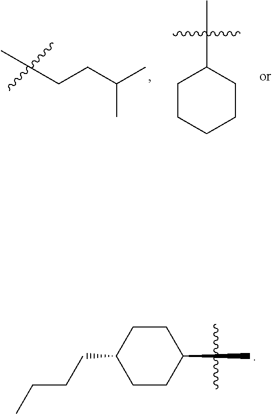

In any of the embodiments described herein, unless otherwise specified or directly contradictory from context, the abiraterone prodrug can be abiraterone decanoate.

In some embodiments, a once a month or once in more than a month dosing is desired, e.g., the dosing frequency ranges from once a month to once every few months, such as from once a month to once every two months, or from once a month to once every three months. In such embodiments, the abiraterone prodrug needs to not only release abiraterone slowly but also to release abiraterone in a sufficient plasma concentration such that it can be beneficial to the human subject user. The once a month or once in more than a month dosing is typically a parenteral administration, such as intramuscularly, intradermally, or subcutaneously. In any of the embodiments herein, unless directly contradictory, the administration can be an intramuscular administration.

The abiraterone prodrugs and abiraterone prodrug formulations of the present disclosure can be administered to a human subject in need thereof as the only source of abiraterone. However, in some embodiments, other abiraterone medications/formulations are not excluded. For example, in some embodiments, the administering herein can be combined, either concurrently or sequentially in any order, with an oral administration of abiraterone acetate, such as the Zytiga® formulation. In some embodiments, the human subject can use the abiraterone prodrugs and abiraterone prodrug formulations as a supplement to an existing abiraterone therapy. Moreover, the administering herein is not limited to administering a single abiraterone prodrug or abiraterone prodrug formulation of the present disclosure. In some embodiments, two or more abiraterone prodrugs and abiraterone prodrug formulations of the present disclosure can be administered to the human subject.

In some embodiments, prior to a once a month or once in more than a month dosing, the method herein can include an initial treatment period with a higher dosing frequency, such as a once a week or once in two weeks dosing. The initial treatment period can include administering the same abiraterone prodrug or a different abiraterone medication such as a different abiraterone prodrug. In some embodiments, the method herein does not include such initial treatment period.

As discussed herein, the abiraterone prodrugs and abiraterone prodrug formulations of the present disclosure have many advantages over the currently marketed Zytiga® product. For example, administering the abiraterone prodrugs and abiraterone prodrug formulations of the present disclosure to a human subject typically results in reduced Cₘₐₓ of abiraterone (e.g., reduced by at least 30% compared to the Cₘₐₓ of abiraterone observed at steady state for a once daily oral dose of Zytiga® at 1000 mg without food).

Thus, in some embodiments, the present disclosure provides a method of treating subjects having side effects related to high abiraterone exposure, such as having abiraterone Cₘₐₓ related side effects, the method comprising administering abiraterone prodrugs and abiraterone prodrug formulations of the present disclosure to the human subject, wherein the administering reduces the side effects when compared to administering of a once daily oral dose of Zytiga® at 1000 mg without food. Suitable routes of administration, dosing amounts, frequencies include those described herein. Various side effects or adverse effects are described in the Zytiga® prescribing information approved by the FDA, see e.g., the February 2018 or June 2019 version. In some embodiments, the present disclosure provides a method of treating subjects who are also administered a drug, the metabolism of which is inhibited by abiraterone, for example, drugs that are CYP2D6 and/or CYP2C8 substrates, the method comprising administering to the human subject the abiraterone prodrugs and abiraterone prodrug formulations of the present disclosure, wherein the administering reduces the inhibition of the metabolism of the drug when compared to administering of a once daily oral dose of Zytiga® at 1000 mg without food. In some embodiments, the present disclosure provides a method of treating a human subject who has, or is at risk of having, hypertension, hypokalemia, or fluid retention due to mineralocorticoid excess, the method comprising administering to the human subject the abiraterone prodrugs and abiraterone prodrug formulations of the present disclosure, wherein the administering reduces hypertension, hypokalemia, and fluid retention or the risk of hypertension, hypokalemia, and fluid retention when compared to administering of a once daily oral dose of Zytiga® at 1000 mg without food. In some embodiments, the present disclosure provides a method of treating a human subject who has, or is at risk of having, adrenocortical insufficiency, the method comprising administering to the human subject the abiraterone prodrugs and abiraterone prodrug formulations of the present disclosure, wherein the administering reduces adrenocortical insufficiency or the risk of having adrenocortical insufficiency when compared to administering of a once daily oral dose of Zytiga® at 1000 mg without food. In some embodiments, the present disclosure provides a method of treating a human subject who has severe or fatal hepatotoxicity after taking Zytiga®, the method comprising administering to the human subject the abiraterone prodrugs and abiraterone prodrug formulations of the present disclosure, wherein the administering reduces hepatotoxicity. Without wishing to be bound by theories, it is believed that administering the abiraterone prodrugs and abiraterone prodrug formulations of the present disclosure typically results in a reduced, yet efficacious abiraterone exposure and therefore is beneficial for subjects who need a lower dose of abiraterone, e.g., as described above. Suitable dosing regimens, routes of administrations include those described herein.

### Exemplary Methods Using Abiraterone Decanoate

In some specific embodiments, the present disclosure also provides a method of treating a sex hormone-dependent or androgen receptor driven disease or disorder, such as a sex hormone-dependent benign or malignant disorder, an androgen receptor driven cancer, and/or a syndrome due to androgen excess, comprising administering to a human subject in need thereof a therapeutically effective amount of the pharmaceutical composition comprising abiraterone decanoate described herein (e.g., any of those described in the Summary section herein, such as [18]-[30] or [93]-[107] of the Summary section herein, or any of those described in the Examples section). The administering is not limited to any particular route. However, the abiraterone decanoate is typically administered parenterally, for example, via an intramuscular injection, intradermal injection, or subcutaneous injection. In some embodiments, the administering is through intramuscular injection. Unlike oral administration of abiraterone acetate, the pharmaceutical composition comprising abiraterone decanoate described herein (e.g., the unit dosage form described herein) can be administered to the human subject in need with or without food. In some embodiments, the human subject is a non-castrated human subject. In some embodiments, the human subject is a castrated human subject. In some embodiments, the method herein can also administer the pharmaceutical composition comprising abiraterone decanoate to the human subject without regard to whether the human subject is castrated or not. In some embodiments, the method herein can also advantageously treat human subjects suffering from hepatic impairment, such as moderate to severe hepatic impairment (Child-Pugh Class B or C), prior to the administering of the abiraterone decanoate. In some embodiments, the method herein can also advantageously treat a human subject having prostate cancer, and the method herein does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition. In some embodiments, the method herein is for treating a human subject having prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition herein.

Sex hormone-dependent benign or malignant disorder that can be treated with the methods include any of those described herein such as a sex hormone dependent cancer. In some embodiments, the sex hormone-dependent benign or malignant disorders can be selected from androgen-dependent disorders and estrogen-dependent disorders such as androgen-dependent or estrogen-dependent cancers. In some embodiments, the sex hormone-dependent benign or malignant disorders can be selected from prostate cancer, breast cancer, ovarian cancer, bladder cancer, hepatocellular carcinoma, and lung cancer, etc. In some embodiments, the sex hormone-dependent benign or malignant disorder can be prostate cancer or breast cancer. In some embodiments, the sex hormone-dependent benign or malignant disorder is CRPC or CSPC. In some embodiments, the sex hormone-dependent benign or malignant disorder can be metastatic CRPC or metastatic CSPC. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. Syndromes due to androgen excess that can be treated with the methods include any of those described herein. In some embodiments, the method herein can be a method for treating a non-oncologic syndrome in the human subject due to androgen excess, such as endometriosis, polycystic ovary syndrome, congenital adrenal hyperplasia (e.g., classical or nonclassical congenital adrenal hyperplasia), precocious puberty, hirsutism, etc.

In some specific embodiments, the method is for treating a sex hormone dependent or androgen receptor driven cancer, such as prostate cancer (e.g., described herein), androgen receptor positive salivary duct carcinoma, or androgen receptor positive glioblastoma multiforme.

The method herein can be used in conjunction with one or more additional therapies for the respective disease or disorder. For example, the method can comprise administering one or more other drug or agent (for example, as described herein, such as another cancer chemotherapeutic drug, hormone replacement drug, or hormone ablation drug) to the human subject, either concurrently or sequentially, through the same route or a different route of administration. Non-limiting examples of useful additional therapies also include any of those described in [37]-[49] in the Summary section herein.

In some embodiments, the method herein (e.g., treating a prostate cancer, or treating classical or nonclassical congenital adrenal hyperplasia) can comprise administering to the human subject an agent that offsets the reduction of glucocorticoid(s). In some embodiments, the method herein can comprise administering to the human subject in need an agent effective in treating one or more symptoms associated with adrenal insufficiency, such as acute stress, fatigue, etc. In some specific embodiments, the method herein can comprise administering to the human subject a steroid, such as a corticosteroid. In some embodiments, the method can comprise administering to the human subject a glucocorticoid. In some specific embodiments, the method also comprises administering to the human subject prednisone, prednisolone, and/or methylprednisolone. In some specific embodiments, the method comprises administering to the human subject hydrocortisone, prednisone, prednisolone, methylprednisolone, and/or dexamethasone. In some embodiments, the method also comprises administering to the human subject an agent effective in treating cortisol deficiency, for example, hydrocortisone, prednisone, prednisolone, methylprednisolone, and/or dexamethasone. In any such embodiments, the agent can be administered to the human subject either concurrently or sequentially in any order, via a same or different route of administration, in reference to the administration of abiraterone decanoate.

In some embodiments according to the method herein, the abiraterone prodrug, in particular, abiraterone decanoate, is administered to the human subject in combination with dexamethasone. For example, in any of the methods described herein, unless specified or otherwise contrary from context, the method can comprise administering abiraterone decanoate and dexamethasone to the human subject. In some embodiments, the method is for treating prostate cancer, such as for treating a human subject who is a chemotherapy naïve CRPC patient, including a chemotherapy naïve mCRPC patient. In some embodiments, the method can comprise administering to the human subject (i) abiraterone decanoate intramuscularly once every one to three months, preferably, about 1260 mg of abiraterone decanoate once every three months; and (ii) dexamethasone orally once daily. In some embodiments, the dexamethasone is orally administered to the human subject at a dose of about 0.1 to 1 mg/day, such as about 0.5 mg/day. Additional therapies can also be used in combination with the abiraterone decanoate and dexamethasone treatment, which without limitation include any of such other therapies described herein, such as any of those applicable therapies described in [37]-[49] in the Summary section herein.

However, in some embodiments, a glucocorticoid replacement therapy (e.g., administering a glucocorticoid, such as hydrocortisone, prednisone, prednisolone, methylprednisolone, or dexamethasone) is not desired. For example, a glucocorticoid may be contraindicated for the human subject, who may have an underlying condition, such as diabetics. In some embodiments, the method can also be characterized in that the human subject is not treated with a glucocorticoid replacement therapy. In some embodiments, the human subject is not treated with an agent selected from hydrocortisone, prednisone, prednisolone, methylprednisolone, and dexamethasone. In some embodiments, the method can comprise administering to the human subject a mineralocorticoid receptor antagonist, such as eplerenone. For example, in any of the embodiments herein when glucocorticoid replacement therapy is not desired and/or not administered, the method can comprise administering to the human subject a mineralocorticoid receptor antagonist, such as eplerenone.

In some embodiments, the method herein does not cause a reduction of glucocorticoid levels such that a glucocorticoid replacement therapy is not required. In such embodiments, the human subject can avoid using glucocorticoid replacement therapy and/or mineralocorticoid receptor antagonist, even if glucocorticoid is not contraindicated for the human subject.

In some embodiments, the method herein does not cause a significant increase in mineralocorticoid levels and/or does not cause a toxicity due to mineralocorticoid excess. In some embodiments, the method herein is characterized in that an agent for treating a mineralocorticoid toxicity is not needed and not administered to the human subject. In some embodiments, the method herein is characterized in that the human subject is not treated with an agent effective for treating a mineralocorticoid toxicity. In some embodiments, the method herein is characterized in that the human subject is not treated with an agent that is a glucocorticoid or a mineralocorticoid receptor antagonist.

In some embodiments, the method is for treating prostate cancer and includes a combination therapy, which further comprising administering to the human subject one or more additional therapies, e.g., as described herein under the section titled Combination Treatment for Prostate Cancer. Non-limiting examples of useful additional therapies also include any of those described in [37]-[49] in the Summary section herein.

In some embodiments, the method herein can be characterized by a dosing frequency of once a week or even less frequent. Typically, the dosing frequency can range from once a week to once every few months, such as from once a week to once every three months, or from once a week to once every eight weeks, such as once a month, once every two months, or once every three months. In some embodiments, the method comprises administering to the human subject the pharmaceutical composition comprising abiraterone decanoate (e.g., the unit dosage form described herein) once a week, once in two weeks, once in three weeks, once a month, or once in more than a month such as once every two months, or once every three months. In some embodiments, the method comprises administering to the human subject the pharmaceutical composition comprising abiraterone decanoate (e.g., the unit dosage form described herein) once in two weeks, once a month, or once in more than a month, e.g., once every two months, or once every three months. In some embodiments, the dosing amount for each dose is about 50 mg to about 5000 mg (e.g., about 100 mg, about 350 mg, about 500 mg, about 1000 mg, about 1500 mg, about 2000 mg, about 5000 mg, or any ranges between the recited values) of abiraterone decanoate. In some embodiments, the dosing amount of abiraterone decanoate for each dose is about 0.5 mg/kg to about 200 mg/kg (e.g., about 0.5 mg/kg, about 1 mg/kg, about 5 mg/kg, about 10 mg/kg, about 20 mg/kg, about 30 mg/kg, about 50 mg/kg, about 90 mg/kg, about 100 mg/kg, about 200 mg/kg, or any ranges between the recited values) of body weight of a human subject. In some embodiments, the administering is via intramuscular injection. In some embodiments, the administering of a single dose provides a therapeutically effective blood plasma concentration of abiraterone a period of at least one week, e.g., at least two weeks, such as at least three weeks, at least four weeks, and up to six or eight weeks or more, such as up to ten weeks or more, etc. In some embodiments, the administering of a single dose provides a blood plasma concentration of abiraterone above 1.0 ng/ml (e.g., between about 1 ng/ml and about 8 ng/ml, or about 2 ng/ml or higher, about 4 ng/ml or higher, about 5 ng/ml or higher, or about 8 ng/ml or higher) for a period of at least one week, e.g., at least two weeks, such as at least 3 weeks, at least four weeks, and up to six or eight weeks or more, such as up to ten weeks or more, etc. In some embodiments, the administering provides a single dose or steady state Cₘₐₓ of abiraterone between about 1 ng/ml and about 300 ng/ml, for example, between about 1 ng/ml and about 10 ng/ml. In some embodiments, the administering can also provide a concentration of abiraterone in a tissue of the human subject at least 10 times higher than the blood plasma concentration of abiraterone at 7 days post administration (i.e., at 168 hours from the time of administration), wherein the tissue is selected from liver, lung, testes, inguinal lymph, iliac lymph, adrenal, and prostate. In some embodiments, the abiraterone decanoate formulation can be administered to the human subject in need thereof as the only source of abiraterone. However, in some embodiments, the abiraterone decanoate formulation can also be administered to the human subject in need thereof as a supplement to another abiraterone therapy.

In some embodiments, in particular in the methods of treating prostate cancer herein, the dosing amount and frequency of abiraterone decanoate can be adjusted such that the administering provides an effective amount of abiraterone to reduce the serum testosterone level to about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, within 15 days (e.g., within 7 days, between 7-15 days, etc.) of the first administration of the abiraterone decanoate. For example, in some embodiments, the administering provides an effective amount of abiraterone to reduce the serum testosterone level to about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, when measured on day 15 after the first administration of the abiraterone decanoate. In some embodiments, the administering provides an effective amount of abiraterone to achieve a sustained reduction of serum testosterone level, such as achieving and maintaining the serum testosterone level at about 50 ng/dL or below (e.g., about 40 ng/dL or below, about 30 ng/dL or below, about 20 ng/dL or below, about 10 ng/dL or below, etc.) within 15 days (e.g., within 7 days, between 7-15 days, etc.) in a non-castrated human subject or about 1 ng/dL or below in a castrated human subject, of the first administration of the abiraterone decanoate. In some embodiments, the dosing amount and frequency of abiraterone decanoate can be adjusted such that the administering provides an effective amount of abiraterone to reduce 50% or more, preferably, 75% or more of serum testosterone level from baseline within 15 days (e.g., within 7 days, between 7-15 days, etc.) of the first administration of the abiraterone decanoate. For example, in some embodiments, the administering provides an effective amount of abiraterone to reduce 50% or more, preferably, 75% or more of serum testosterone level from baseline when measured on day 15 after the first administration of the abiraterone decanoate. In some embodiments, the administering provides an effective amount of abiraterone to achieve a sustained reduction of serum testosterone level, such as by 50% or more, 75% or more, from baseline within 15 days (e.g., within 7 days, between 7-15 days, etc.) of the first administration of the abiraterone decanoate.

In some specific embodiments, the present disclosure provides a method of treating a sex hormone-dependent or androgen receptor driven disease or disorder, such as a sex hormone-dependent benign or malignant disorder or a syndrome due to androgen excess (e.g., any of those described herein), in a human subject in need thereof, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values. In some embodiments, the administering of the abiraterone decanoate selectively inhibits CYP17A1 lyase activity over CYP17A1 hydroxylase activity in the human subject. In some embodiments, the sex hormone-dependent or androgen receptor driven disease or disorder, such as a sex hormone-dependent benign or malignant disorder or syndrome due to androgen excess, is a disease or disorder in human subjects where lyase selectivity is desired or deemed beneficial, such as endometrial cancer, endometriosis, ovarian cancer, and prostate cancer (such as localized prostate cancer, etc.).

In some specific embodiments, the present disclosure provides a method of treating prostate cancer (e.g., any of those described herein) in a human subject in need thereof, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values. In some embodiments, the prostate cancer is CRPC or CSPC. In some embodiments, the prostate cancer is metastatic CRPC or metastatic CSPC. In some embodiments, the prostate cancer is a localized prostate cancer. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. Non-limiting examples of prostate cancer also include any of those described in [8]-[17] in the Summary section herein. In some embodiments, the method of treating prostate cancer includes a combination therapy, which further comprises administering to the human subject one or more additional therapies, e.g., as described herein under the section titled Combination Treatment for Prostate Cancer. For example, in some embodiments, the human subject is treated with an androgen receptor antagonist, e.g., enzalutamide, such as administering enzalutamide to the human subject orally once a day at a daily dose of about 160 mg. In some embodiments, the method comprises administering abiraterone decanoate and dexamethasone to the human subject, such as a human subject who is a chemotherapy naïve CRPC patient, including a chemotherapy naïve mCRPC patient. In some embodiments, the method can comprise administering to the human subject (i) abiraterone decanoate intramuscularly once every one to three months, preferably, about 1260 mg of abiraterone decanoate once every three months; and (ii) dexamethasone orally once daily. In some embodiments, the dexamethasone is orally administered to the human subject at a dose of about 0.1 to 1 mg/day, such as about 0.5 mg/day. Non-limiting examples of useful additional therapies also include any of those described in [37]-[49] in the Summary section herein. However, in some embodiments, an agent for treating a mineralocorticoid toxicity is not needed and not administered to the human subject. In some embodiments, the human subject is not treated with an agent effective for treating a mineralocorticoid toxicity. In some embodiments, the human subject is not treated with an agent that is a glucocorticoid or a mineralocorticoid receptor antagonist.

In some specific embodiments, the present disclosure provides a method of treating a cancer in a human subject in need thereof, wherein the cancer is a sex hormone dependent or androgen receptor driven cancer, which has metastasized to one or more lymph nodes, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values. In some embodiments, the administering of the abiraterone decanoate is effective in inhibiting growth of the cancer in the one or more lymph nodes. In some embodiments, the cancer is prostate cancer (e.g., any of those described herein). For example, in some embodiments, the prostate cancer is CRPC or CSPC. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. Non-limiting examples of prostate cancer also include any of those described in [8]-[17] in the Summary section herein. In some embodiments, the method includes a combination therapy, which further comprises administering to the human subject one or more additional therapies, e.g., as described herein under the section titled Combination Treatment for Prostate Cancer. For example, in some embodiments, the human subject is treated with an androgen receptor antagonist, e.g., enzalutamide, such as administering enzalutamide to the human subject orally once a day at a daily dose of about 160 mg. In some embodiments, the method comprises administering abiraterone decanoate and dexamethasone to the human subject, such as a human subject who is a chemotherapy naïve CRPC patient, including a chemotherapy naïve mCRPC patient. In some embodiments, the method can comprise administering to the human subject (i) abiraterone decanoate intramuscularly once every one to three months, preferably, about 1260 mg of abiraterone decanoate once every three months; and (ii) dexamethasone orally once daily. In some embodiments, the dexamethasone is orally administered to the human subject at a dose of about 0.1 to 1 mg/day, such as about 0.5 mg/day. Non-limiting examples of useful additional therapies also include any of those described in [37]-[49] in the Summary section herein. However, in some embodiments, an agent for treating a mineralocorticoid toxicity is not needed and not administered to the human subject. In some embodiments, the human subject is not treated with an agent effective for treating a mineralocorticoid toxicity. In some embodiments, the human subject is not treated with an agent that is a glucocorticoid or a mineralocorticoid receptor antagonist.

In some specific embodiments, the present disclosure provides a method of treating a cancer in a human subject in need thereof, wherein the cancer is a sex hormone dependent or androgen receptor driven cancer, wherein the human subject's disease has progressed on or after an androgen receptor antagonist based treatment, such as enzalutamide based treatment, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values. In some embodiments, the cancer is prostate cancer (e.g., any of those described herein). For example, in some embodiments, the prostate cancer is CRPC or CSPC. In some embodiments, the prostate cancer is metastatic CRPC or metastatic CSPC. In some embodiments, the prostate cancer is a localized prostate cancer. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. Non-limiting examples of prostate cancer also include any of those described in [8]-[17] in the Summary section herein. In some embodiments, the method includes a combination therapy, which further comprises administering to the human subject one or more additional therapies, e.g., as described herein under the section titled Combination Treatment for Prostate Cancer. For example, in some embodiments, the human subject is treated with an androgen receptor antagonist, e.g., enzalutamide, such as administering enzalutamide to the human subject orally once a day at a daily dose of about 160 mg. In some embodiments, the method comprises administering abiraterone decanoate and dexamethasone to the human subject, such as a human subject who is a chemotherapy naïve CRPC patient, including a chemotherapy naïve mCRPC patient. In some embodiments, the method can comprise administering to the human subject (i) abiraterone decanoate intramuscularly once every one to three months, preferably, about 1260 mg of abiraterone decanoate once every three months; and (ii) dexamethasone orally once daily. In some embodiments, the dexamethasone is orally administered to the human subject at a dose of about 0.1 to 1 mg/day, such as about 0.5 mg/day. Non-limiting examples of useful additional therapies also include any of those described in [37]-[49] in the Summary section herein. However, in some embodiments, an agent for treating a mineralocorticoid toxicity is not needed and not administered to the human subject. In some embodiments, the human subject is not treated with an agent effective for treating a mineralocorticoid toxicity. In some embodiments, the human subject is not treated with an agent that is a glucocorticoid or a mineralocorticoid receptor antagonist.

In some specific embodiments, the present disclosure provides a method of treating a cancer in a human subject in need thereof, wherein the cancer is a sex hormone dependent or androgen receptor driven cancer, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values, wherein the human subject is further treated with an androgen receptor antagonist, such as enzalutamide, prior to, concurrent, or subsequent to the administration of the abiraterone decanoate. In some embodiments, the human subject is further treated with enzalutamide. In some embodiments, the enzalutamide can be administered to the human subject orally once a day at a daily dose of about 160 mg. In some embodiments, the enzalutamide treatment can start at least one day prior to the first administration of the abiraterone decanoate. In some embodiments, the enzalutamide treatment can start on the same day of the first administration of the abiraterone decanoate. In some embodiments, the enzalutamide treatment can start at least one day after the first parenteral administration of the abiraterone decanoate. In some embodiments, the androgen receptor antagonist(s) can be administered to the human subject in combination with the parenteral administration of the pharmaceutical composition to achieve a synergistic effect on cancer treatment. In some embodiments, the cancer is prostate cancer (e.g., any of those described herein). For example, in some embodiments, the prostate cancer is CRPC or CSPC. In some embodiments, the prostate cancer is metastatic CRPC or metastatic CSPC. In some embodiments, the prostate cancer is a localized prostate cancer. In some embodiments, the prostate cancer is CRPC, in some embodiments, the patient having CRPC is chemotherapy naïve. In some embodiments, the prostate cancer is a metastatic CRPC (mCRPC), in some embodiments, the patient having mCRPC is chemotherapy naïve. Non-limiting examples of prostate cancer also include any of those described in [8]-[17] in the Summary section herein. In some embodiments, the method includes a combination therapy, which further comprises administering to the human subject one or more additional therapies, e.g., as described herein under the section titled Combination Treatment for Prostate Cancer. For example, in some embodiments, the human subject is treated with an androgen receptor antagonist, e.g., enzalutamide, such as administering enzalutamide to the human subject orally once a day at a daily dose of about 160 mg. In some embodiments, the method comprises administering abiraterone decanoate and dexamethasone to the human subject, such as a human subject who is a chemotherapy naïve CRPC patient, including a chemotherapy naïve mCRPC patient. In some embodiments, the method can comprise administering to the human subject (i) abiraterone decanoate intramuscularly once every one to three months, preferably, about 1260 mg of abiraterone decanoate once every three months; and (ii) dexamethasone orally once daily. In some embodiments, the dexamethasone is orally administered to the human subject at a dose of about 0.1 to 1 mg/day, such as about 0.5 mg/day. Non-limiting examples of useful additional therapies also include any of those described in [37]-[49] in the Summary section herein. However, in some embodiments, an agent for treating a mineralocorticoid toxicity is not needed and not administered to the human subject. In some embodiments, the human subject is not treated with an agent effective for treating a mineralocorticoid toxicity. In some embodiments, the human subject is not treated with an agent that is a glucocorticoid or a mineralocorticoid receptor antagonist.

In some specific embodiments, the present disclosure provides a method of reducing serum testosterone level in a human subject in need thereof, the method comprising the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values, wherein the administering of the abiraterone decanoate does not enhance serum progesterone level in the human subject (i) by more than 40% above baseline at 4 weeks following the first administration of the abiraterone decanoate; and/or (ii) by more than 40% above baseline from 2 weeks to 12 weeks following the second administration of the abiraterone decanoate, preferably, does not enhance serum progesterone level in the human subject (i) by more than 20% above baseline at 4 weeks following the first administration of the abiraterone decanoate, (ii) by more than 20% above baseline at 6 weeks, 8 weeks, 10 weeks, and/or 12 weeks following the first administration of the abiraterone decanoate; and/or (iii) by more than 20% above baseline from 2 weeks to 12 weeks following the second administration of the abiraterone decanoate. In some embodiments, the abiraterone decanoate is administered in an effective amount to achieve a sustained reduction of serum testosterone level in the human subject to 50% below baseline or lower within 15 days of the first administration of the abiraterone decanoate. In some embodiments, the abiraterone decanoate is administered in an effective amount to reduce the serum testosterone level to about 50 ng/dL or below (e.g., about 40 ng/dL or below, about 30 ng/dL or below, about 20 ng/dL or below, about 10 ng/dL or below, etc.), when the human subject is a non-castrated human subject or about 1 ng/dL or below, when the human subject is a castrated human subject, when measured on day 15 after the first administration of the abiraterone decanoate. In some embodiments, the abiraterone decanoate is administered in an effective amount to reduce the serum testosterone level in the human subject to 80% below baseline or lower, such as 85% below baseline or lower, or 90% below baseline or lower, when measured at 24 weeks after the first administration of the abiraterone decanoate. In some embodiments, the abiraterone decanoate is administered in an effective amount to reduce the serum testosterone level to about 50 ng/dL or below (e.g., about 40 ng/dL or below, about 30 ng/dL or below, about 20 ng/dL or below, about 10 ng/dL or below, etc.), when the human subject is a non-castrated human subject, or about 1 ng/dL or below, when the human subject is a castrated human subject, when measured at 24 weeks after the first administration of the abiraterone decanoate. In some embodiments, the abiraterone decanoate is administered in an effective amount to achieve a sustained reduction of serum testosterone level, such as by 50% or more, 75% or more, from baseline within 15 days (e.g., within 7 days, between 7-15 days, etc.) of the first administration of the abiraterone decanoate. In some embodiments, the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the abiraterone decanoate. In some embodiments, the sustained reduction of serum testosterone level is characterized in that once the serum testosterone level in the human subject about 50 ng/dL or below (e.g., about 40 ng/dL or below, about 30 ng/dL or below, about 20 ng/dL or below, about 10 ng/dL or below, etc.) in a non-castrated human subject or about 1 ng/dL or below in a castrated human subject, the serum testosterone level remains at about 50 ng/dL or below in a non-castrated human subject or about 1 ng/dL or below in a castrated human subject up to 8 weeks or longer following the first administration of the the abiraterone decanoate. In some embodiments, the human subject suffers from prostate cancer (e.g., any of those described herein).

In some specific embodiments, the present disclosure also provides a method of inhibiting CYP17A1 lyase activity in a human subject in need thereof, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values, wherein the administering of the abiraterone decanoate does not enhance serum progesterone level in the human subject (i) by more than 40% above baseline at 4 weeks following the first administration of the abiraterone decanoate; and/or (ii) by more than 40% above baseline from 2 weeks to 12 weeks following the second administration of the abiraterone decanoate, preferably, does not enhance serum progesterone level in the human subject (i) by more than 20% above baseline at 4 weeks following the first administration of the abiraterone decanoate, (ii) by more than 20% above baseline at 6 weeks, 8 weeks, 10 weeks, and/or 12 weeks following the first administration of the abiraterone decanoate; and/or (iii) by more than 20% above baseline from 2 weeks to 12 weeks following the second administration of the abiraterone decanoate. In some embodiments, the human subject suffers from a sex hormone-dependent or androgen receptor driven disease or disorder. In some embodiments, the human subject suffers from a sex hormone-dependent benign or malignant disorder, e.g., as described herein. In some embodiments, the human subject suffers from a syndrome due to androgen excess, e.g., as described herein.

In some specific embodiments, the present disclosure also provides a method of reducing the level of androgens (e.g., testosterone and/or dihydrotestosterone) and/or estrogens in a human subject in need thereof in a human subject in need thereof, the method comprising administering abiraterone decanoate to the human subject intramuscularly, at a dosing frequency of once every 1-3 months, such as once every three months, wherein each administration comprises administering to the human subject about 50 mg to about 2000 mg of abiraterone decanoate, such as about 180 mg, about 360 mg, about 720 mg, about 1260 mg, about 1800 mg, or any range between the recited values, wherein the administering of the abiraterone decanoate does not enhance serum progesterone level in the human subject (i) by more than 40% above baseline at 4 weeks following the first administration of the abiraterone decanoate; and/or (ii) by more than 40% above baseline from 2 weeks to 12 weeks following the second administration of the abiraterone decanoate, preferably, does not enhance serum progesterone level in the human subject (i) by more than 20% above baseline at 4 weeks following the first administration of the abiraterone decanoate, (ii) by more than 20% above baseline at 6 weeks, 8 weeks, 10 weeks, and/or 12 weeks following the first administration of the abiraterone decanoate; and/or (iii) by more than 20% above baseline from 2 weeks to 12 weeks following the second administration of the abiraterone decanoate. In some embodiments, the human subject suffers from a syndrome due to androgen excess, such as congenital adrenal hyperplasia (e.g., classical or nonclassical congenital adrenal hyperplasia), endometriosis, polycystic ovary syndrome precocious puberty, hirsutism, etc. In some embodiments, the human subject suffers from an androgen and/or estrogen associated cancer, such as prostate cancer or breast cancer. In some embodiments, the human subject suffers from an androgen receptor driven cancer, such as those described herein.

Human subjects suitable to be treated with the method herein comprising administering abiraterone decanoate are not particularly limited. In some embodiments, the human subject can be a non-castrated human subject. In some embodiments, the human subject can also be castrated. For example, in some embodiments, the human subject can be chemically castrated, such as treated with a gonadotropin-releasing hormone agonist and/or antagonist. In some embodiments, the human subject can be characterized as suffering from hepatic impairment, such as moderate to severe hepatic impairment (Child-Pugh Class B or C), prior to the administering of the abiraterone decanoate. In some embodiments, the method herein can also advantageously treat a human subject having prostate cancer, and the method herein does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition. In some embodiments, the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition herein. In some embodiments, the human subject can be characterized as being sensitive to or otherwise intolerant with a gonadotropin-releasing hormone antagonist and/or agonist. In some embodiments, the human subject can be characterized as chemotherapy naïve or hormone therapy naïve prior to being administered the abiraterone decanoate. However, in some embodiments, the human subject can also be treated with chemotherapy or hormone therapy prior to being administered abiraterone decanoate. For example, in some embodiments, the human subject can have a disease or disorder (e.g., prostate cancer) that has progressed on or after the chemotherapy and/or hormone therapy, such as a taxane-based chemotherapy regimen, for example, docetaxel-based or cabazitaxel-based chemotherapy. In some embodiments, the human subject's disease has progressed on or after an androgen receptor antagonist based treatment, such as enzalutamide based treatment. In some embodiments, the human subject's disease has progressed on or after an oral abiraterone acetate based treatment, such as oral abiraterone acetate and prednisone based treatment. For example, in some embodiments, the human subject has developed resistance to the treatment of abiraterone acetate in combination with prednisone, including resistance due to increased levels of progesterone. In some embodiments, the human subject can be characterized as suffering from one or more side effects associated with inhibition of CYP17A1 hydroxylase activity.

In some preferred embodiments, the method herein can comprise administering abiraterone decanoate to the human subject intramuscularly, at a dosing frequency of once every three months, wherein each administration comprises administering to the human subject about 1260 mg of abiraterone decanoate.

In any of the embodiments described herein, unless specified or otherwise contrary, the abiraterone decanoate can be formulated in a pharmaceutical composition, which comprises, for each milliliter, (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); and (d) corn oil, q.s. to 1 milliliter. In any of the embodiments described herein, unless specified or otherwise contrary, the abiraterone decanoate can be formulated in a pharmaceutical composition comprises an abiraterone decanoate solution, wherein each milliliter of the abiraterone decanoate solution comprises, consists essentially of, or consists of: (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 20 mg (e.g., about 0.5 mg, about 1 mg, about 2 mg, or about 5 mg); and (e) corn oil, q.s. to 1 milliliter. In some embodiments, the weight ratio of benzyl alcohol to benzyl benzoate in the pharmaceutical composition ranges from about 2:1 to about 1:5 (e.g., about 1:1 to 1:3, such as about 1:2). In some embodiments, the pharmaceutical composition is characterized as having (1) a viscosity of less than 0.1 Pa*s, such as about 0.05 Ps*s or lower; (2) a glide force of about 1-10 N when measured using a 21G, 1.5 inch needle, and/or about 2-15 N when measured using a 23 gauge (or 23G), 1.5 inch needle, and/or about 30-150 N when measured using a 27G, 1.5 inch needle; (3) no more than 1000 particles having a size of 10 m or greater, and no more than 300 particles having a size of 25 m or greater, when measured according to USP <788> and/or <789>; and/or (4) less than 100 EU/ml, such as less than 25 EU/ml of bacterial endotoxins measured according to USP <85>. Methods for measuring viscosity and glide force are known in the art, which are also exemplified in Example 2 herein. The USP methods <788>, <789> and <85> referenced herein should be understood as the current version of such methods, which are also known by those skilled in the art.

In any of the embodiments described herein, unless specified or otherwise contrary, the abiraterone decanoate can be formulated in a pharmaceutical composition according to any of [18]-[30] or [93]-[107] of the Summary section herein.

In any of the embodiments described herein, unless specified or otherwise contrary, the abiraterone decanoate can be formulated in a pharmaceutical composition with ingredients on a per milliliter basis according to those shown in the examples, such as Example 2 or Example 4 herein. For example, in some embodiments, the pharmaceutical composition comprises, per 1 milliliter, abiraterone decanoate about 200 mg, benzyl alcohol about 100 mg, benzyl benzoate about 200 mg, and corn oil q.s. to 1 mL; or Abiraterone Decanoate, about 180 mg; Benzyl Alcohol, about 100 mg; Benzyl Benzoate, about 200 mg; and Corn oil, q.s. to 1 mL. In some embodiments, the pharmaceutical composition comprises, per 1 milliliter, (a) abiraterone decanoate in its basic form, in an amount of about 180 mg; (b) benzyl alcohol in an amount of about 100 mg; (c) benzyl benzoate in an amount of about 200 mg; (d) 3-mercapto-1,2-propanediol in an amount of about 0.5 mg to about 2 mg (e.g., about 0.5 mg, about 1 mg, or about 2 mg); and (e) corn oil, q.s. to 1 milliliter.

Provided herein are formulations, methods, and kits for treating a human subject with a sex hormone-dependent or androgen receptor driven disease or disorder, such as a sex hormone-dependent benign or malignant disorder such as prostate cancer. Also provided are methods for preparing the formulations useful for treating a human subject with a sex hormone-dependent or androgen receptor driven disease or disorder, such as a sex hormone-dependent benign or malignant disorder (such as prostate cancer), an androgen receptor driven cancer, and/or a syndrome due to androgen excess. Reference will now be made in detail to representative embodiments, examples of which are illustrated in the accompanying drawings.

The term “human subject” as used herein means, but is not limited to, a human in need of or capable of receiving chemotherapy for a sex hormone-dependent or androgen receptor driven disease or disorder, such as a sex hormone-dependent benign or malignant disorder such as, for example, an androgen-dependent disorder or an estrogen-dependent disorder (including prostate cancer and breast cancer), an androgen receptor driven cancer, a human in need of or capable of receiving therapy for non-oncologic syndromes due to androgen excess, such as endometriosis, polycystic ovary syndrome, congenital adrenal hyperplasia (e.g., classical or nonclassical congenital adrenal hyperplasia), precocious puberty, hirsutism, etc.

The term “other drug or agent” as used herein (when, for example, referring to prior, simultaneous, and post-administration of at least one other drug or agent with at least one abiraterone prodrug formulation) means at least one other compound, formulation, molecule, biologic, or the like, capable of enhancing the efficacy of the formulation(s), decreasing an undesirable side effect(s) of the formulation(s), or improving the treatment of the particular disorder. Any suitable routes of administration of such “other drug or agent” can be used, for example, oral administration, parenteral administration, etc. A person skilled in the art of treating a human subject having a sex hormone-dependent or androgen receptor driven disease or disorder, such as a sex hormone-dependent benign or malignant disorder (such as an androgen-dependent disorder or an estrogen-dependent disorder), an androgen receptor driven cancer, and/or syndromes due to androgen excess syndrome would know and understand how to choose and use such “other drug or agent” for the intended purpose(s).

The formulations can optionally be administered via a modified-release device or method. The term “modified-release” as used herein should be understood as encompassing delayed release, prolonged or extended release, sustained release, or a targeted release, etc. For example, in some embodiments, the modified release device or method can further prolong the release of abiraterone of the prodrugs and formulations of the present disclosure. In some embodiments, the modified release device or method can also include any device or method capable of releasing an agent or product (for example, a drug or a biologic) at a time later than immediately following its administration (and can include, for example, implants). Various modified release devices have been described (Stubbe et al., Pharm. Res. 21:1732, 2004) and could be applicable to the representative embodiments. Modified-release devices and methods can be identified and employed without undue experimentation by a person skilled in the art after consideration of all criteria and use of best judgment on the human subject's behalf.

The formulations and agents of the embodiments are administered in a pharmacologically or physiologically acceptable and effective amount to reduce or eliminate the presence, for example, of prostate tumor tissue and abnormal or malignant prostate cells in a human subject presenting with prostate cancer. Similarly, the formulations and agents of the embodiments are administered alone or in combination with other therapeutic agents or therapeutic modalities (for example, radiotherapy and surgery) in prophylactically or therapeutically effective amounts, which are to be understood as amounts meeting the intended prophylactic or therapeutic objectives and providing the benefits available from administration of such formulations and agents.

The terms “effective amount,” “effective dose,” and “therapeutic blood plasma concentration” as used herein mean, but are not limited to, an amount, dose, or concentration capable of treating, delaying, slowing, inhibiting, or eliminating the onset, existence or progression of a disorder, disease or condition. For example, an “effective amount,” “effective dose,” or “therapeutic blood plasma concentration” is capable of reducing or eliminating the presence of prostate tumor tissue and abnormal or malignant prostate cells in a human subject presenting with prostate cancer, which is sufficient to cure (partly or completely) illness or prevent the onset or further spread of disorder, disease or condition. For further example, an effective amount of formulation refers to the amount administered alone or in combination with other therapeutic agents or therapeutic modalities (for example, radiotherapy and surgery) to achieve clinically significant reduction in tumor burden. A person skilled in the art would understand when a clinically significant reduction in tumor burden (or improvement of a sex hormone-dependent or androgen receptor driven disease or disorder, such as a sex hormone-dependent benign or malignant disorder or another disorder or syndrome described herein) has occurred following administration of a formulation. An “effective amount,” “effective dose,” or “therapeutic blood plasma concentration” is understood to be an amount, dose, or concentration not critically harmful to the human subject and, in any case, where any harmful side effects are outweighed by benefits. By way of example only, an effective amount or dose of an abiraterone prodrug formulation means an amount capable of attaining blood plasma concentrations of at least 0.1 ng/ml, e.g., at least 0.5 ng/ml, at least 1 ng/ml, at least 2 ng/ml, at least 4 ng/ml, or at least 8 ng/ml, of abiraterone in the human subject following parenteral administration of the prodrug formulation, and the efficacious blood plasma concentrations are attained for at least one week, e.g., at least two weeks (for example, four, six, eight or more weeks) following administration.

In general, the dosage ranges for administration of the formulation according to the present disclosure are those that produce the desired effect(s). The useful dosage to be administered will vary depending on the age, weight, and health of the human subject treated, the mode, route, and schedule of administration, the response of the individual subject, and the type or staging of prostate cancer (or severity of a sex hormone-dependent or androgen receptor driven disease or disorder, such as a sex hormone-dependent benign or malignant disorder or another syndrome or disorder described herein) against which treatment with the formulation is sought. The dosage will also vary with the nature or the severity of the primary tumor and other underlying conditions, with epidemiologic conditions, with the concomitant use of other active compounds, and the route of administration. In addition, the dosage will be determined by the existence of any adverse side effects such as local hypersensitivity, systemic adverse effects, and immune tolerance.

An effective dose of the formulations (and other agent(s)) can be determined without undue experimentation (for example, by pharmacokinetic studies) by a person skilled in the art after consideration of all criteria and use of best judgment on the patient's behalf (and will most often be contingent upon the particular formulation utilized). The dosage to be administered will depend upon the particular case, but in any event, it is the amount sufficient to induce clinical benefit against, or improvement of, a sex hormone-dependent or androgen receptor driven disease or disorder, such as a sex hormone-dependent benign or malignant disorder (such as prostate cancer), an androgen receptor driven cancer, and/or a syndrome due to androgen excess.

The formulations and agents of the embodiments can, optionally, be administered in combination with (or can include) one or more pharmaceutically acceptable carriers, diluents, or excipients. Formulations, administration techniques, pharmaceutical compositions, methods of preparing pharmaceutical compositions, and pharmaceutically acceptable carriers, diluents, and excipients are known in the art and are described, for example, in “Remington: The Science and Practice of Pharmacy” (formerly “Remington's Pharmaceutical Sciences,” University of the Sciences in Philadelphia, Lippincott, Williams & Wilkins, Philadelphia, Pa. (2005)), the disclosure of which is hereby incorporated by reference. A person skilled in the art can use known injectable, physiologically acceptable sterile solutions. For preparing a ready-to-use solution for parenteral injection or infusion, aqueous isotonic solutions, for example, saline, phosphate buffered saline (PBS) or corresponding plasma protein solutions, are readily available. The formulations can be present as lyophylisates or dry preparations, which can be reconstituted with a known injectable solution directly before use under sterile conditions, for example, as a kit of parts. In addition, the formulations can include one or more acceptable carriers (which can include, for example, solvents, dispersion media, coatings, adjuvants, stabilizing agents, diluents, preservatives, antibacterial and antifungal agents, isotonic agents, absorption-modifying agents, and the like. “Diluents” can include water, saline, phosphate-buffered saline (PBS), dextrose, ethanol, glycerol, and the like. Isotonic agents can include sodium chloride, dextrose, mannitol, sorbitol, and lactose, among others. Stabilizers include albumin and alkali salts of ethylenediaminetetraacetic acid, among others.

Any suitable route of administration can be employed for providing a human subject with an effective amount/dosage of formulation and agents according to the representative embodiments. A suitable route of administration can be determined readily by a person skilled in the art of pharmacology, immunology, medicine, oncology, or the like without undue experimentation. However, it is anticipated that the formulations are primarily suitable for parenteral administration such as via TM injection, intradermal injection, or subcutaneous injection.

The abbreviations used herein have their conventional meaning within the chemical and biological arts.

As used herein, the singular form “a”, “an”, and “the”, includes plural references unless it is expressly stated or is unambiguously clear from the context that such is not intended. For example, unless specified or otherwise contrary from context, when referring to “the method herein”, it should be understood as including any of the applicable methods described herein.

Headings and subheadings are used for convenience and/or formal compliance only, do not limit the human subject technology, and are not referred to in connection with the interpretation of the description of the human subject technology. Features described under one heading or one subheading of the human subject disclosure may be combined, in various embodiments, with features described under other headings or subheadings. Further it is not necessarily the case that all features under a single heading or a single subheading are used together in embodiments.

As used herein, the term “about” modifying an amount related to the disclosure refers to variation in the numerical quantity that can occur, for example, through routine testing and handling; through error in such testing and handling; through differences in the manufacture, source, or purity of ingredients/materials employed in the disclosure; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

It is also meant to be understood that a specific embodiment of a variable moiety herein may be the same or different as another specific embodiment having the same identifier.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5′ Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

As used herein, the term “alkyl” as used by itself or as part of another group refers to a straight- or branched-chain saturated aliphatic hydrocarbon. In some embodiments, the alkyl can include one to thirty carbon atoms (i.e., C₁₋₃₀ alkyl or alternatively expressed as C₁-C₃₀ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₆ alkyl group. In another embodiment, the alkyl group is a branched chain C₃₋₁₆ alkyl group. To be clear, when a range of carbon numbers is listed, it encompasses each individual integer within the range and sub-ranges between such integers as would be understood by those skilled in the art. For example, “C₇₋₁₆” herein encompasses, C₇, C₈, C₉, C₁₀, C₁₁, C₁₂, C₁₃, C₁₄, C₁₅, C₁₆, C₇₋₁₆, C₇₋₁₅, C₇₋₁₄, C₇₋₁₃, C₇₋₁₂, C₇₋₁₁, C₇₋₁₀, C₇₋₉, C₇₋₈, C₈₋₁₆, C₈₋₁₅, C₈₋₁₄, C₈₋₁₃, C₈₋₁₂, C₈₋₁₁, C₈₋₁₀, C₈₋₉, C₉₋₁₆, C₉₋₁₅, C₉₋₁₄, C₉₋₁₃, C₉₋₁₂, C₉₋₁₁, C₉₋₁₀, C₁₀₋₁₆, C₁₀₋₁₅, C₁₀₋₁₄, C₁₀₋₁₃, C₁₀₋₁₂, C₁₀₋₁₁, C₁₁₋₁₆, C₁₁₋₁₅, C₁₁₋₁₄, C₁₁₋₁₃, C₁₁₋₁₂, C₁₂₋₁₆, C₁₂₋₁₅, C₁₂₋₁₄, C₁₂₋₁₃, C₁₃₋₁₆, C₁₃₋₁₅, C₁₃₋₁₄, C₁₄₋₁₆, C₁₄₋₁₅, and C₁₅₋₁₆. Other ranges as described herein such as “number of carbons between 5 and 16” etc. should be understood similarly.

As used herein, the term “cycloalkyl” as used by itself or as part of another group refers to saturated and partially unsaturated (e.g., containing one or two double bonds) cyclic aliphatic hydrocarbons containing one to three rings having from three to twelve carbon atoms (i.e., C₃₋₁₂ cycloalkyl) or the number of carbons designated. In one embodiment, the cycloalkyl group has two rings. In one embodiment, the cycloalkyl group has one ring. In another embodiment, the cycloalkyl group is a C₃₋₈ cycloalkyl group. In another embodiment, the cycloalkyl group is a C₃₋₆ cycloalkyl group. “Cycloalkyl” also includes ring systems wherein the cycloalkyl ring, as defined above, is fused with one or more aryl or heteroaryl groups wherein the point of attachment is on the cycloalkyl ring, and in such instances, the number of carbons continue to designate the number of carbons in the cycloalkyl ring system. Non-limiting exemplary cycloalkyl groups include cyclopropyl, cyclobutyl, cyclopentyl, cyclohexyl, cycloheptyl, cyclooctyl, norbornyl, decalin, adamantyl, cyclopentenyl, and cyclohexenyl.

As used herein, the term “alkenyl” as used by itself or as part of another group refers to a straight- or branched-chain aliphatic hydrocarbon containing one or more (e.g., 1, 2, or 3) carbon-to-carbon double bonds. In one embodiment, the alkenyl group is a C₂₋₁₆ alkenyl group.

As used herein, the term “alkynyl” as used by itself or as part of another group refers to a straight- or branched-chain aliphatic hydrocarbon containing one or more (e.g., 1, 2, or 3) carbon-to-carbon triple bonds. In one embodiment, the alkynyl has one carbon-carbon triple bond. In one embodiment, the alkynyl group is a C₂₋₁₆ alkynyl group.

As used herein, the term “abiraterone prodrug(s) of the present disclosure” refers to any of the compounds described herein according to Formula I or II, a lipophilic ester of abiraterone prodrug, isotopically labeled compound(s) thereof (e.g., deuterium enriched compounds), possible stereoisomers thereof (including diastereoisomers, enantiomers, and racemic mixtures), tautomers thereof, conformational isomers thereof, and/or pharmaceutically acceptable salts thereof (e.g., acid addition salt such as HCl salt). Hydrates and solvates of the prodrugs of the present disclosure are considered compositions of the present disclosure, wherein the prodrug(s) is in association with water or solvent, respectively. Some of the prodrugs of the present disclosure can also exist in various polymorphic forms or amorphous forms. The prodrugs described herein include those compounds that readily undergo chemical changes under physiological conditions to provide active abiraterone. Additionally, prodrugs can be converted by chemical or biochemical methods in an ex vivo environment. As used herein, the term “abiraterone prodrug formulation(s) of the present disclosure” refers to any of the pharmaceutical composition or formulation comprising any one or more of the abiraterone prodrugs of the present disclosure, for example, any of the formulations prepared in Example 2 or 4. In any of the embodiments described herein, unless directly contradictory from context, the abiraterone prodrug of the present disclosure can be abiraterone decanoate. In any of the embodiments described herein, unless directly contradictory from context, the abiraterone prodrug formulation of the present disclosure can be any of the pharmaceutical composition comprising abiraterone decanoate as described herein.

The abiraterone prodrugs of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, oxygen, and nitrogen, include, but are not limited to ²H, ³H, ¹³C ¹⁴C, ¹⁵N, and ¹⁸O. Compounds that contain other isotopes of these and/or other atoms are within the scope of this disclosure.

Solid and dashed wedge bonds indicate stereochemistry as customary in the art.

The following examples are provided for illustration purposes only and are in no way intended to limit the scope of the claimed subject matter.

### Example 1a. Large Scale Preparation of Abiraterone Decanoate from Decanoic Acid

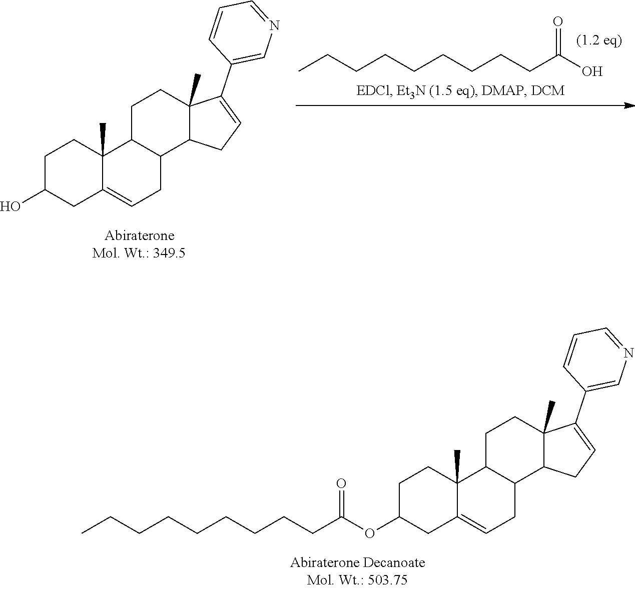

To a suspension of Abiraterone (381.9 g, 1.09 mol) in dichloromethane (3500 mL) was added triethylamine (165 g, 1.64 mol) and a catalytic amount of DMAP (13.35 g, 0.109 mol). Decanoic acid (225 g, 1.31 mol) as a solution in dichloromethane (500 mL) was added to the suspension, followed by EDCI (293 g, 1.53 mol) and the reaction then agitated for 19 h at 20-25° C.

10 wt % aq NaH₂PO₄ (4000 mL) was then added and the reaction was agitated for 20 min. The organic layer was separated and extracted with 10 wt % aq NaH₂PO₄ (2000 mL) and brine (2000 mL). The organic layer was solvent exchanged with acetonitrile (4750 mL) and concentrated to 3100 g keeping temperature of bath <40° C. The suspension was diluted with acetonitrile (900 g). The solids were isolated by filtration to afford 510 g of crude abiraterone decanoate.

510 g of the crude abiraterone decanoate was dissolved in acetone (4000 mL) at 40° C. The solution was filtered through a filter paper. The filtrate was transferred to a 12 L 3-neck flask, diluted to 5100 g and reheated to 40° C. to form a solution. The solution was cooled slowly to 20° C. to form a suspension. This was diluted with water (1020 mL) and agitated at RT overnight. The solid was filtered and the flask was rinsed with the filtrate and transferred to filter funnel. The wet cake was transferred to drying tray and dried at 40-45° C. in vacuum oven overnight to obtain 457.1 g (90% yield) as white solid, the crystalline form of this solid is designated as Form A. ¹H NMR (CDCl₃, 400 MHz): d_{H} 8.62 (d, 1H, J=1.9 Hz), 8.31 (dd, 1H, J=4.9, 1.6 Hz), 7.64 (dt, 1H, J=7.9, 1.9 Hz), 7.21 (ddd, 1H, J=8.0, 4.9, 0.8 Hz), 6.01-5.97 (m, 1H), 5.44-5.40 (m, 1H), 4.68-4.58 (m, 1H), 2.39-2.23 (m, 3H), 2.27 (t, 2H, J=7.6 Hz), 2.12-2.00 (m, 3H), 1.91-1.54 (m, 10H), 1.49 (dt, 1H, J=11.9, 5.1 Hz), 1.35-1.23 (m, 12H), 1.20-1.07 (m, 2H), 1.08 (s, 3H), 1.05 (s, 3H), 0.88 (t, 3H, J=6.8 Hz). Elemental Analysis, theoretical (corrected for 0.055% moisture level): C, 81.0%, H, 9.8%, N, 2.8%; found: C, 81.1%, H, 10.2%, N, 2.8%. Differential Scanning Calorimetry (DSC) pattern of this solid shows an endothermic peak with an onset temperature at about 69.0° C.

The abiraterone decanoate obtained in this example was determined to have a purity of 99.7% by weight using a HPLC method. For HPLC analysis, abiraterone decanoate samples were prepared in methanol at a concentration of 0.05 mg/mL (for assay analysis) or 5 mg/mL (for impurity analysis). The HPLC conditions are the following: HPLC column: Halo C8 (2.7 um, 100×3.0 mm); injection volume: 5 uL; Column Temperature: 40° C.; Sample Temperature: ambient; Detection: 210 nm; Mobile Phase: 25 mM Ammonium Acetate, pH 8.0 (MPA) and 95/5 acetonitrile/tetrahydrofuran (MPB); Flow Rate: 0.6 ml/min; Gradient: starting with 65/35 MPA/MPB, in 35 minutes, reaching to 100% MPB, hold at 100% MPB until 40 minutes, at 40.10 minute, back to 65/35 MPA/MPB, and hold at 65/35 MPA/MPB until end at 45 minutes.

The white solid obtained in this example was also characterized by X-Ray Powder Diffraction (XRPD) and Differential Scanning Calorimetry (DSC). XRPD was conducted with Bruker's D8 Discover X-rat diffractometer, with Theta\theta vertical goniometer, using Vantec-500 as detector. Standard conditions: voltage 40 kV, current 40 mA, radiation, Cu, temperature, ambient, X-ray source exit slit size, 0.5 mm pinhole, snout collimator, 0.5 mm, sample holder, ground quartz plate. Operating conditions: detector distance, 30 cm, Chi integration range, 4- to 40-degree 20, count time, 120 seconds/frame, # of frames: 3, Theta 1 position, 4 degree, Theta 2 position, 4 degree, Frame width, 12, scan axis, coupled. Software used include GADDs software, General Area Detector Diffraction System, version 4.1.50; and DIFFRAC.EVA, version 4.0. DSC was performed with TA Instruments Q2000 (Thermal Advantage V 5.0.0—qualified), with a sample size of 2-10 mg, heating range from 25° C. to 250° C. at a heating rate of 10° C./min. A thermogravimetric analysis (TGA) was also performed on this sample. TGA was performed with TA Instruments TGA Q500 (Thermal Advantage V5.2.5—qualified), with a sample size of 5-20 mg, heating range from 25° C. to 150° C. at a heating rate of 10° C./min. Representative XRPD, DSC, and TGA spectra of Form A of abiraterone decanoate are shown in U.S. Pat. No. 10,792,292.

### Example 1B. Preparation of High Purity Abiraterone Decanoate

This example shows a process of purifying abiraterone decanoate to remove residue palladium. Abiraterone decanoate used for this Example was prepared using similar procedures as shown in Example 1A.

Crude abiraterone decanoate (7.17 kg) was dissolved in acetone (142 kg) at room temperature. Activated carbon (1.43 kg) was added and the resulting slurry stirred at room temperature for 4 hours. The mixture was filtered to remove the activated carbon and the solids were washed with acetone (142 kg). The combined acetone filtrates were concentrated by vacuum distillation at 40° C. The concentrated filtrates, which contained about 72 L acetone, were then cooled to 20° C. and water (4.3 kg) was slowly added. The mixture was stirred at 20° C. for 12 hours and the abiraterone decanoate was collected by filtration. The product was washed with 1:1 acetone/water (7.2 kg) and dried under vacuum at 40° C. to yield 5.524 kg of pure abiraterone decanoate (Form A). Analytical data are consistent with those described in Example 1A. A representative certificate of analysis of the obtained abiraterone decanoate is shown in Table D below. Purity of the obtained abiraterone decanoate was analyzed using HPLC Method 1, see details in Example 2 below.

TABLE D
Specification and Analysis of Abiraterone Decanoate

| Test Method | Specifications | Result |
|---|---|---|
| Appearance | White to yellow solid | White Solid |
| Identification by IR-ATR | Sample spectrum is | Conforms |
| | consistent with | |
| | Abiraterone Decanoate | |
| | reference standard | |
| | spectrum | |
| Abiraterone Decanoate Content by HPLC (% w/w) | 98-102 (dried basis) | 99.6 |
| Related Substances by HPLC (% w/w) | | |
| Abiraterone | NMT 0.5 | nd |
| Ethylprasterone Decanoate | NMT 0.5 | 0.4 |
| Any Unspecified Impurity | NMT 0.1 | nd |
| Total Impurities | NMT 1.0 | 0.4 |
| Residual Solvents by Gas Chromatography (ppm) | | |
| Dichloromethane | NMT 600 | 181 |
| Acetonitrile | NMT 410 | nd |
| Acetone | NMT 5000 | nd |
| Residual EDU (EDCI coupling reagent by- | NMT 0.15 | <0.05 |
| product) by LC-MS (% w/w) | | |
| Residual Decanoic Acid by Gas Chromatography | NMT 5000 | NT |
| (ppm) | | |
| Triethylamine by Ion Chromatography (% w/w) | NMT 0.5 | <0.1 |
| Palladium by ICP (ppm) | NMT 10 | 3.7 |
| Water by Karl Fischer (% w/w) | NMT 1.0 | 0.12 |
| Residue on Ignition (% w/w) | NMT 0.5 | <0.05 |
| Physical Form by XRPD | Report Results | Crystalline |
| Microbial Enumeration by USP <61> | | |
| Total Aerobic Microbial Count (TAMC) CFU/g | ≤100 | <50 |
| Total Combined Yeasts and Molds (TYMC) | ≤10 | <50 |
| CFU/g | | |
| Bacterial Endotoxins by USP <85> EU/g | ≤350 | <51.4 |

Notes:
nd = not detected.
NT = Not Tested.
NMT = No More Than

The crude abiraterone decanoate contained 130 ppm Pd. Recrystallization from just acetone/water lowered the Pd level to 120 ppm. However, by using the process described in this example, the final abiraterone decanoate can be purified to have a Pd content of only 3.7 ppm.

### Example 2. Preparation of Abiraterone Decanoate Formulations

Approximately a half of the required amount of corn oil was added to a suitable container (˜1,750 ml). The required amount of benzyl alcohol (360 g) was weighed and added to the corn oil. The required amount of benzyl benzoate (720 g) was weighed and added to the corn oil. This mixture was mixed using an appropriate mixer (e.g., shaft mixer) for a minimum of 10 minutes or until all the benzyl alcohol and benzyl benzoate was in solution. The appropriate amount of the abiraterone decanoate (720 g) was weighed out and added to the solution of corn oil/benzyl alcohol/benzyl benzoate and mixed using an appropriate mixer (e.g., shaft mixer) for a minimum of 30 minutes or until all the abiraterone decanoate was in solution. The resulting solution was than diluted to its final volume (3,600 ml) with corn oil to make a solution with the composition given below:

TABLE 1A
Ingredients of Abiraterone Decanoate Formulation

| | | Amount (grams) for a |
|---|---|---|
| Ingredient | Amount (mg) per mL | 3.6 L batch* |
| Abiraterone Decanoate | 200 | 720 |
| Benzyl Alcohol | 100 | 360 |
| Benzyl Benzoate | 200 | 720 |
| Corn Oil | QS to 1 mL | QS to 3,600 mL |

*This is a representative batch size and the batch size can vary based on the amount of drug product needed.

Drug Substance Analysis: The abiraterone decanoate used for preparing the formulations above was obtained from a process similar to those described in Example 1A, except without the recrystallization step. The abiraterone decanoate typically has a purity of 99% by weight (as measured by HPLC) or higher. A typical batch of abiraterone decanoate has a quality as shown in FIG. 3, using HPLC Method 1. Based on such, the assigned purity of such abiraterone decanoate batch is about 99.4% by weight, calculated by the following method: 100%−(% HPLC impurities+% Karl Fischer Moisture+% residue solvents). The HPLC method used for measuring the purity of abiraterone decanoate can be HPLC Method 1: Separation is performed with an Advanced Materials Technology Halo C8 reversed phase column using dimensions of 3.0×100 mm and a particle size of 2.7 μm. A linear gradient program (20 minutes) is used with mobile phases consisting of a 25 mM aqueous ammonium acetate buffer and a mixture of methanol and acetonitrile (see gradient profile below in Table 1B). Working standard and sample solutions are prepared in a methanol diluent. The typical injection volume is 5 μL and the detection wavelength is 210 nm.

TABLE 1B
Gradient Profile of HPLC Method 1
Mobile Phase A:
25 mM Ammonium Acetate in 90% water, 10% Methanol
Mobile Phase B:
25 mM Ammonium Acetate in 90% acetonitrile, 10% Methanol

| Time (min) | Mobile Phase A (%) | Mobile Phase B (%) |
|---|---|---|
| 0 | 50 | 50 |
| 20 | 5 | 95 |
| 25 | 5 | 95 |
| 25.1 | 50 | 50 |
| 30 | 50 | 50 |

Under sterile conditions the final solution is then sterilized by passing the solution through a 0.22-micron PVDF filter using a standard pump system (e.g., peristaltic pump) and placed into to sterile vials (219 vials, 15 ml fill volume). The filled vials are sealed with a rubber stopper and then capped to ensure the integrity of the final product. The fill volume and size of the vial can vary based on the dose to be manufactured.

### Analytical Methods of Abiraterone Decanoate Formulation

Assay, related substances, and identification by retention time of the abiraterone decanoate formulation were conducted using a reversed phase high performance liquid chromatographic analytical method, HPLC Method 2. HPLC Method 2: separation is performed with an XBridge Shield RP18 reversed phase column using dimensions of 4.6×100 mm and a particle size of 3.5 μm. A linear gradient program (25 minutes) is used with mobile phases consisting of a 40 mM aqueous ammonium bicarbonate buffer and a mixture of methanol and acetonitrile (see gradient profile below in Table 1C). Working standard and sample solutions are prepared in isopropyl alcohol diluent. The typical injection volume is 10 μL and the detection wavelength is 254 nm.

TABLE 1C
Gradient Profile of HPLC Method 2
Mobile Phase A:
50:25:25 40 mM Ammonium Bicarbonate Buffer:McOH:ACN
Mobile Phase B:
50:50 MeOH:ACN

| Time (min) | Mobile Phase A (%) | Mobile Phase B (%) |
|---|---|---|
| 0 | 100 | 0 |
| 25 | 0 | 100 |
| 35 | 0 | 100 |
| 35.1 | 100 | 0 |
| 40 | 100 | 0 |

Glide Force Determination: The analytical method is performed using a tensile and compression testing instrument (eg. Lloyd press or equivalent), with a 250N load cell and Nexygen Plus materials testing software. Two separate syringe/needle configurations were used for the analytical measurements (5-mL Luer-Lok syringe configured with a 23 gauge (23G) 1.5-inch thin wall precision glide needle and a 5-mL Luer-Lok syringe configured with a 27 gauge (27G) 1.5 inch regular wall precision glide needle. The glide force measurements are taken using a 5-mL sample size and a constant compression rate.

Viscosity: The analytical method is performed using a Malvern Kinexus Lab+ viscometer instrument with rSpace Rheometry software. The following parameters were developed for the viscosity measurements of the drug product:
Bob Geometry: C25 DIN Splined
Cup Geometry: C25 DIN AL
Analysis Temp: 20.0° C.
Manual Gap: 1.0 mm
Shear Rate: 50s⁻¹
Test time: 1 minute
Sampling Interval: 15 seconds

Particulates: The number of particles in the drug products was measured according to the current version of USP<788> and/or <789>.

Bacterial Endotoxins: The bacterial endotoxins test was performed according to the current version of USP<85>.

### Analytical Results

The table below shows the analytical results using the methods above.

TABLE 2
Representative Analytical Results of Abiraterone Decanoate Formulation

| Test Description | Analytical Results |
|---|---|
| Appearance | Clear glass vial with red flip-off cap, metal |
| | overseal and rubber stopper containing a clear |
| | yellow solution free of visible particulates |
| Identification by HPLC | Conforms |
| Chromatographic Purity by HPLC (% of | 98.9 |
| Label Claim, 200 mg/ml)) | |
| Related Substances by HPLC (% w/w) | |
| RRT 1.19 | 0.11 |
| RRT 1.21 | 0.13 |
| RRT1.39 | 0.12 |
| Total Impurities | 0.4 |
| Viscosity (Pa*s) | 0.05420 |
| Glide Force (N) | |
| 23 G 1.5-inch needle | 7.6962 |
| 27 G 1.5-inch needle | 70.326 |
| Particulates (HIAC) | |
| Particles ≥10 μm | 352 |
| Particles ≥25 μm | 68 |
| Bacterial Endotoxins by USP <85> EU/mL | <25 |

The impurity having a relative retention time of 1.19 was determined to be

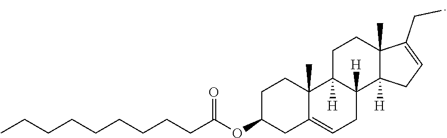

Following similar procedures discussed above, abiraterone decanoate formulation having Abiraterone Decanoate at a concentration of about 180 mg/mL was also prepared using the abiraterone decanoate prepared according to Example 1B. The ingredients of the 180 mg/mL formulation include the following, for each milliliter: Abiraterone Decanoate, about 180 mg/mL; Benzyl Alcohol, about 100 mg/mL; Benzyl Benzoate, about 200 mg/mL; and Corn oil, q.s. to 1 mL.

The glide force of the 180 mg/mL abiraterone decanoate formulation (see formulation details above) was also tested using two separate syringe/needle configurations: 5-mL Luer-Lok syringe configured with a 23 gauge (23G) 1.5-inch thin wall precision glide needle and a 5-mL Luer-Lok syringe configured with a 21 gauge (21G) 1.5-inch regular wall precision glide needle. The glide force measurements are taken using a 5-mL sample size and a constant compression rate. The mean glide forces observed for the 180 mg/mL abiraterone decanoate formulation are the following: 3.2835 (for 21G, 1.5-inch needle) and 6.7863 (for 23G, 1.5-inch needle). The glide force measurements were also taken using a 2-mL fill for a 3 mL syringe. Under these settings, the mean glide forces observed for the 180 mg/mL abiraterone decanoate formulation are the following: 1.0957 (for 21G, 1.5-inch needle) and 2.1481 (for 23G, 1.5-inch needle).

Both the 200 mg/mL and 180 mg/mL abiraterone decanoate formulations were found to be storage stable at 25° C./60% RH and 40° C./75% RH for at least 3 months.

### Example 3. Open Label Study of Intramuscular Abiraterone Decanoate in Patients with Advanced Prostate Cancer

This is a Phase 1/2a, Open-label, Multicenter Study of Intramuscular Abiraterone Decanoate Depot in Patients with Advanced Prostate Cancer.

Formulations and dosing regimen: The formulation used in the study (abiraterone decanoate for intramuscular [i.m.] injection) is a solution formulation of abiraterone decanoate at a concentration of 180 mg/mL and will be provided as a 10 mL vial containing 990 mg of abiraterone decanoate in 5.5 mL of solution. See further details of the formulations in Example 2 (for each milliliter: Abiraterone Decanoate, about 180 mg/mL; Benzyl Alcohol, about 100 mg/mL; Benzyl Benzoate, about 200 mg/mL; and Corn oil, q.s. to 1 mL). Abiraterone Decanoate will be administered as an i.m. injection every 84 days (±3 days). Dose escalation will proceed with a modified Fibonacci sequence. Based on emerging data, intermediate doses may be evaluated.

| Cohort | Dose | Volume |
|---|---|---|
| 1 | 180 mg | 1.0 mL |
| 2 | 360 mg | 2.0 mL |
| 3 | 720 mg | 4.0 mL |
| 4 | 1260 mg | 7.0 mL |
| 5 | 1800 mg | 10.0 mL |

The recommended Phase 2 dose (RP2D) for Phase 2a investigation will be a dose from Phase 1 that does not exceed the maximum tolerated dose (MTD) and adequately suppresses serum testosterone (i.e., to ≤1 ng/dL or greater than 90% reduction from baseline at 24 weeks).

The primary objectives of the studies are to evaluate the safety and tolerability of Abiraterone Decanoate, and to determine a preliminary recommended Phase 2 dose (RP2D) of Abiraterone Decanoate depot that does not exceed the MTD and provides adequate testosterone suppression over the course of treatment. For Phase 2a, the primary objectives are to characterize the proportion of patients with testosterone ≤1 ng/dL or a ≥90% reduction from baseline at 24 weeks. In both phases, the pharmacokinetic profile and pharmacodynamic effects of abiraterone decanoate are also evaluated. Secondary objectives for phase 2a also include: (1) To evaluate the preliminary efficacy of Arm A (PRL-02+dexamethasone) and Arm B (abiraterone acetate+prednisone) by comparing the following: (i) Achievement of serum testosterone levels ≤1 ng/dL or a ≥90% reduction from baseline by day 28; (ii) The time course and change in serum testosterone levels to ≤1 ng/dL or a ≥90% reduction from baseline; (iii) Duration of serum testosterone levels ≤1 ng/dL or a 90% reduction from baseline; (2) Response as defined by any of the outcomes listed below. If any of these occur, the subject will be considered to have responded: (i) Objective response per RECIST v1.1 with a minimum interval for confirmation of CR and PR of 4 weeks; (ii) PSA decline of ≥50% from baseline, confirmed by a second consecutive PSA assessment at least 3 weeks later; (iii) Conversion of circulating tumor cell count (CTC) to <5 cells/7.5 mL blood nadir confirmed by an additional assessment at least 3 weeks later (for subjects with a CTC count of ≥5 cells/7.5 mL blood at baseline); and (iv) Radiographic progression-free survival (rPFS); (3) Overall survival (OS) defined as the time from the first dose of study drug to the date of death due to any cause; (4) Best Overall Response (BOR) per RECIST v1.1, defined as the best radiographic response across all time-point responses; and (5) DOR (defined as the length of time from date of first documented, confirmed response) using CTC and/or PSA and/or RECIST v1.1 and PCWG3 until date of documented progression or death from any cause.

Study Design: This is a Phase 1/2a, Open-label, Multicenter Study of Intramuscular Abiraterone Decanoate Depot in Patients with Advanced Prostate Cancer. In Phase 1, all patients will receive i.m. Abiraterone Decanoate depot in 84-day treatment cycles combined with daily oral prednisone or dexamethasone. In Phase 2a, patients will be randomized 1:1 and treated with i.m. Abiraterone Decanoate depot in 84-day treatment cycles in combination with dexamethasone (Arm A) or abiraterone acetate daily+prednisone (Arm B). In both Phases, patients will undergo scheduled periodic assessments of serum testosterone levels. Participants may continue treatment until they meet any discontinuation criteria outlined in the protocol.

Phase 1 will follow a 3+3 design that is intended to identify the RP2D that does not exceed the MTD and adequately suppresses serum testosterone over the course of treatment. Phase 2a will confirm the safety, tolerability and pharmacodynamic effects of the RP2D selected from Phase 1. In Phase 1 and Phase 2a, patients who meet the criteria for study discontinuation will be contacted 30 days after the end of treatment and every 12 weeks thereafter to collect information on patient disease status, survival status and any subsequent prostate cancer treatment. Survival follow up will continue until death, withdrawal of consent, or the end of the study.

Phase 1—Dose Escalation: Eligible patients with metastatic castration-sensitive prostate cancer (mCSPC) and patients with metastatic castration-resistant prostate cancer (mCRPC) will be enrolled. The initial dose of Abiraterone Decanoate to be administered is based on findings from completed non-human primate toxicology and pharmacology studies; dose escalation will proceed in a modified Fibonacci sequence. A Safety Review Committee (SRC) will be established to review available cohort safety data, and pharmacokinetic and pharmacodynamic findings (particularly testosterone suppression) to recommend a decision on dose escalation including whether intermediate doses should be explored.

At each dose cohort, 3 to 4 patients will be enrolled. If none of the patients experience a dose-limiting toxicity (DLT) in the first 28 days (the DLT period) the dose will be escalated in the next cohort. A DLT will be defined as any of the following using on the Common Terminology Criteria for Adverse Events (CTCAE) criteria v5.0 regardless of attribution to the study drug unless it is clearly due to the patient's underlying disease or extraneous causes:

### Hematologic

≥Grade 3 hematologic toxicity (exception: Grade 3 lymphopenia) Non-hematologic
≥Grade 3 elevations in AST, ALT or bilirubin
Meets drug-induced liver injury (DILI) criteria
Any other ≥Grade 3 non-hematologic toxicity except:
   Grade 3 nausea, vomiting, or diarrhea that persists ≤72 hours with adequate antiemetic and other supportive care
   Grade 3 fatigue that improves to ≤Grade 2 within 5 days
   Grade 3 electrolyte imbalance that corrects within 48 hours to ≤Grade 2
   Other non-hematologic laboratory Grade 3 AEs that are asymptomatic and/or rapidly reversible (returned to baseline or to Grade ≤1 within 7 days) unless identified as clinically relevant by the Investigator.

Patients who prematurely discontinue from a cohort for any reason other than a DLT in the first 28 days will be replaced.

If one of the first 3 to 4 patients in a cohort experiences a DLT, up to 6 patients will be enrolled into that same dose cohort. If no further patients experience a DLT, the dose may be escalated to the next cohort. If more than 1 of 6 patients experience a DLT in a specific dose cohort, the maximum tolerated dose (MTD) will have been exceeded, dose escalation will cease, and the prior dose may be declared the MTD. Alternatively, additional intermediate doses may be explored.

Additional cohort(s) may be added to explore Abiraterone Decanoate in combination with dexamethasone (0.5 mg/day) or in patients not receiving LHRH agonists or antagonists. The additional cohort(s) will operate independently and follow the same rules and schedule of events. Dexamethasone cohort(s) and the cohort of patients not receiving LHRH agonists or antagonists will open at the discretion of the Sponsor. The dose of Abiraterone Decanoate evaluated in the first cohort with dexamethasone or in the cohort the cohort of patients not receiving LHRH agonists or antagonist will be no higher than the dose selected for study in a prednisone cohort, unless a DLT has already been observed in that prednisone cohort in which case the next lowest dose of Abiraterone Decanoate will be selected for initial evaluation of the combination with dexamethasone.

The recommended Phase 2 dose (RP2D) for Phase 2a investigation will be a dose from Phase 1 that does not exceed the maximum tolerated dose (MTD) and adequately suppresses serum testosterone (i.e., to ≤1 ng/dL or ≥90% reduction from baseline) over the course of treatment.

Phase 2a: Eligible patients with mCRPC will be entered into the Phase 2a of the study, an open-label randomized parallel study with up to 48 patients. Patients will be randomized in a 1:1 ratio to receive either Abiraterone Decanoate+dexamethasone (Arm A) or abiraterone acetate+prednisone (Arm B).

Selected Inclusion and Exclusion criteria: Inclusion Criteria include, for example, (1) Written informed consent obtained prior to any study-related procedure being performed. (2) Male patients at least 18 years of age or older at time of consent. (3) Histological evidence of adenocarcinoma of the prostate. (4) For phase 1, patients must have one of the following documented conditions: (a) mCSPC; (b) CSPC with biochemical relapse (using the Prostate Cancer Working Group 3 [PCWG3] definition of PSA progression) of prostate cancer; (c) CSPC with oligometastatic prostate cancer (e.g., Positron Emission Tomography (PET) positive); and (d) mCRPC; and for phase 2a, patients with mCRPC; (5) Undergone orchiectomy or ongoing gonadotropin-releasing hormone (GnRH) agonist or antagonist therapy for at least 1 month prior to the Screening Visit. Exception: for patients being studied in the no concomitant GnRH agonist or antagonist cohort; (6) a serum testosterone level <50 ng/dL but ≥2 ng/dL at screening; (7) Adequate muscle mass for an i.m. injection; (8) An Eastern Cooperative Oncology Group Performance Status (ECOG PS) of 0 or 1; (9) Adequate bone marrow reserve defined as: (a) absolute neutrophil count (ANC) ≥1,500/μL; (b) platelet (PLT) ≥100,000/p L; and (c) hemoglobin (HGB) ≥9 gm/dL; (10) Adequate renal function defined as a serum creatinine ≤1.5×the upper limit of normal (ULN) for the reference lab or a calculated creatinine clearance ≥50 mL/min as determined by a validated algorithm for calculating creatinine clearance; (11) Adequate hepatic function, defined as alanine aminotransferase (ALT) and aspartate aminotransferase (AST)≤2.5×the ULN and total bilirubin ≤1.5×the ULN. Exception for elevated bilirubin secondary to Gilbert's disease. Confirmation of Gilbert's diagnosis requires: elevated unconjugated (indirect) bilirubin values; normal complete blood count in previous 12 months, blood smear, and reticulocyte count; normal aminotransferases and alkaline phosphatase in previous 12 months; (12) Serum albumin ≥3 gm/dL and serum potassium ≥3.5 mEq/L; (13) Patients who are non-sterile and who are heterosexually active with a female partner of childbearing potential must be willing to use a highly effective means of contraception, such as a male condom plus spermicide, from the time of screening, throughout the total duration of the drug treatment, and until 90 days after the final dose of Abiraterone Decanoate; and (14) For Phase 2a only, the patients must have one or more of the following: (a) CTC count of ≥5 cells/7.5 mls blood at screening confirmed by the central laboratory (b) Measurable disease according to RECIST1.1 and a target lesion ≥1 cms in size at screening and (c) PSA value ≥2 μg/L (2 ng/ml) at screening. Exclusion criteria include for example known hypersensitivity to Abiraterone Decanoate, abiraterone, abiraterone decanoate, prednisone or dexamethasone or any of their excipients or components.

Statistical Methods: Descriptive statistics will be used to summarize baseline characteristics, study treatment exposure, safety variables, and preliminary efficacy. Categorical or nominal variables will be summarized by frequency and percentage. Continuous variables will be summarized using standard summary statistics (N, mean, standard deviation, median, minimum, and maximum). For all efficacy endpoints, 95% confidence intervals around point estimates will be presented.

Safety Analysis: Adverse events will be summarized by severity, seriousness, and relationship to study treatments. Laboratory data, vital signs, PFTs and ECGs will be compared to baseline values.

Pharmacokinetic Analysis: Determination of plasma PK parameters will include but may not be limited to maximum (Cₘₐₓ) and minimum (Cₘᵢₙ) observed concentrations, the time to Cₘₐₓ (Tₘₐₓ), the apparent volume of distribution (Vd/F), the apparent systemic clearance (CL/F), various AUC metrics (AUCₗₐₛₜ, AUC_{inf}, AUCₜₐᵤ) and half-life, based on plasma levels of abiraterone, abiraterone decanoate and certain known abiraterone metabolites (i.e., abiraterone sulfate, N-oxide abiraterone, N-oxide abiraterone sulfate, D4 abiraterone, and 3-keto-5-alpha abiraterone.

Pharmacodynamic Analysis: Determination of Prostate Specific Antigen (PSA), serum testosterone, dehydroepiandrosterone (DHEA), dehydroepiandrosterone sulfate (DHEA-S), androstenedione, dihydrotestosterone (DHT), progesterone, cortisol, adrenocorticotropic hormone (ACTH), corticosterone, 11-dexoycorticosterone (11-DOC) and 11-deoxycortisol.

Efficacy Analysis (Phase 2a only): Testosterone responses by treatment arm will be tabulated for achievement of serum testosterone levels ≤1 ng/dL at week 24 or a reduction in testosterone of ≤90% at 24 weeks; the time course and change in serum testosterone levels to ≤1 ng/dL; and duration of serum testosterone levels ≤1 ng/dL. Clinical responses will be tabulated by cohort using the PCWG3 response criteria, and/or RECIST 1.1 and/or PSA response and/or CTC criteria. Summary statistics will be prepared for ORR, DOR, PFS, and OS for patients treated at the RP2D.

Partial Results: while the study is ongoing, the following results show preliminary pharmacokinetic and pharmacodynamic outcomes of a few cohorts and certain patients. Based on the available data, the recommended dose for Phase 2 is about 1260 mg abiraterone decanoate once every three months (12 weeks). All cohorts 1 and 2 in this clinical study were treated with prednisone, 5 mg orally once or twice daily; and all cohorts 3-5 were treated with dexamethasone, 0.5 mg orally once daily. The results shown herein for these cohorts should be understood accordingly.

Serum Steroid Levels: the partial results show that for cohorts 1-5, serum testosterone levels are reduced from baseline, up to 95% in cohort 5 observed on day 168, 80% and 86% reduction were observed for cohorts 3 and 4, respectively. See FIG. 3A and Table 3A below. The median baseline T level, calculated from mean of screening and Day 1 of Cycle 1 pre-dose samples, was 7.45 ng/dL. Among patients dosed at 720 mg and above, 13 out of 16 patients had a 90% reduction in testosterone levels compared to baseline or values reduced to ≤1 ng/dL at day 28.

TABLE 3A
Serum Testosterone Dose Response

| Study | Cohort | 180 mg | Cohort | 360 mg | Cohort | 720 mg | Cohort | 1260 mg | Cohort | 1800 mg |
|---|---|---|---|---|---|---|---|---|---|---|
| Day | 1 (n) | (% CFB) | 2 (n) | (% CFB) | 3 (n) | (% CFB) | 4 (n) | (% CFB) | 5 (n) | (% CFB) |
| 1 | 3 | 0% | 3 | 0% | 4 | 0% | 6 | 0% | 6 | 0% |
| 2 | 3 | −33% | 3 | −11% | 4 | −41% | 6 | −51% | 5 | −41% |
| 3 | 3 | −42% | 3 | 0% | 4 | −50% | 6 | −65% | 5 | −46% |
| 5 | 3 | −48% | 3 | −28% | 4 | −48% | 6 | −69% | 5 | −62% |
| 7 | 3 | −52% | 3 | −49% | 4 | −65% | 6 | −79% | 5 | −76% |
| 10 | 3 | −61% | 3 | −51% | 4 | −74% | 6 | −79% | 5 | −79% |
| 14 | 3 | −67% | 3 | −67% | 4 | −82% | 6 | −80% | 6 | −85% |
| 21 | 3 | −71% | 3 | −72% | 4 | −87% | 6 | −88% | 5 | −88% |
| 28 | 3 | −73% | 3 | −71% | 4 | −85% | 6 | −89% | 6 | −88% |
| 42 | 3 | −65% | 3 | −64% | 4 | −81% | 6 | −90% | 5 | −93% |
| 56 | 3 | −55% | 2 | −60% | 3 | −83% | 6 | −89% | 5 | −79% |
| 70 | 3 | −47% | 2 | −56% | 3 | −81% | 5 | −80% | 6 | −87% |
| 77 | | | 2 | −56% | 3 | −80% | 6 | −87% | 5 | −88% |
| 84 | | | 2 | −49% | 3 | −76% | 6 | −83% | 4 | −88% |
| 112 | | | 1 | −79% | 3 | −94% | 6 | −93% | 3 | −83% |
| 140 | | | 1 | −63% | 3 | −88% | 4 | −89% | 2 | −96% |
| 168 | | | 1 | −32% | 3 | −80% | 3 | −86% | 2 | −95% |
| 196 | | | 1 | −56% | 3 | −93% | 2 | −97% | 2 | −98% |

It was also observed that serum DHEA-S, DHEA, androstenedione, levels were significantly reduced from baseline. See FIGS. 3B, 3C, and 3D.

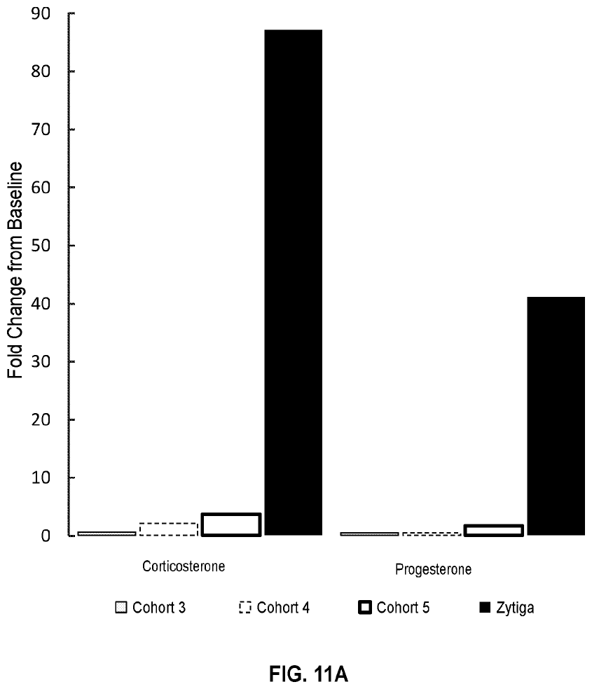

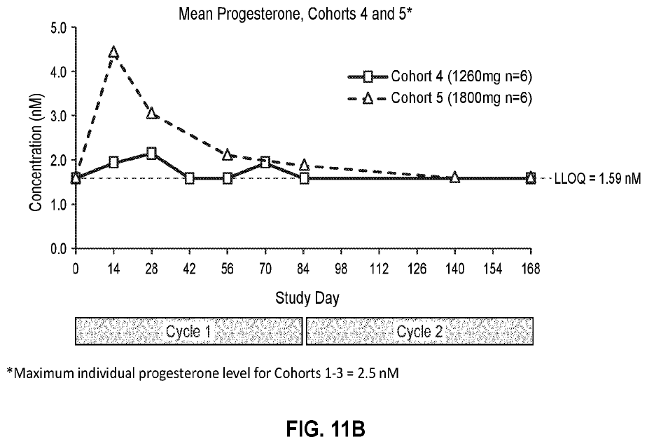

As shown in FIG. 3E, the progesterone changes from baseline are not significant and in all cohorts, returned to baseline quickly. See also Table 3B below. For cohort 5, progesterone level was increased within the first two weeks following the first administration, but returned to baseline around week 6, and during the second cycle, no significant increases of progesterone levels were observed for all cohorts. See also FIG. 11B for the absolute serum progesterone levels for cohorts 4 and 5, with the LLOQ being 1.59 nM. It should be noted that the maximum serum progesterone level observed for cohorts 1-3 is 2.5 nM. FIGS. 3F and 3G show the serum 11-deoxycorticosterone and corticosterone response in cohorts 3, 4, and 5, the trend is similar to that observed for progesterone levels above. See a summary shown in the Table 3C below for the first cycle.

TABLE 3B
Serum Progesterone Dose Response

| Study | Cohort | 180 mg | Cohort | 360 mg | Cohort | 720 mg | Cohort | 1260 mg | Cohort | 1800 mg |
|---|---|---|---|---|---|---|---|---|---|---|
| Day | 1 (n) | (% CFB) | 2 (n) | (% CFB) | 3 (n) | (% CFB) | 4 (n) | (% CFB) | 5 (n) | (% CFB) |
| 1 | 3 | 0% | 3 | 0% | 4 | 0% | 6 | 0% | 6 | 0% |
| 14 | 3 | 0% | 3 | 37% | 4 | −1% | 4 | 20% | 6 | 165% |
| 28 | 3 | 8% | 3 | 8% | 3 | −2% | 6 | 35% | 6 | 86% |
| 42 | | | | | | | 1 | 0% | 2 | 0% |
| 56 | 3 | 0% | 3 | 0% | 3 | 0% | 5 | 0% | 6 | 12% |
| 70 | 2 | 0% | | | | | 3 | 22% | 3 | 0% |
| 84 | | | 2 | 0% | 3 | 0% | 5 | 0% | 5 | 8% |
| 112 | | | | | | | 3 | 6% | | |
| 140 | | | | | | | 1 | 0% | 1 | 0% |
| 168 | | | 1 | 0% | 3 | 6% | 4 | 1% | 2 | −7% |
| 196 | | | | | | | | | 2 | 52% |
| 224 | | | | | 1 | 0% | 1 | 0% | 2 | −7% |
| 252 | | | 1 | 0% | 1 | 0% | 3 | 0% | 2 | 0% |

TABLE 3C
Summary of Progesterone and Corticosterone
Changes during cycle 1
Percent (%)

| | Up-Stream | Day | | | |
|---|---|---|---|---|---|
| Dose Group | Steroids | 14 | 28 | 56 | 84 |
| 720 mg | Progesterone | −1 | −2 | 0 | 0 |
| (N = 4) | Corticosterone | 48 | 27 | 9 | −33 |
| 1,260 mg | Progesterone | 20 | 35 | 0 | 0 |
| (N = 6) | Corticosterone | 184 | 490 | 19 | 32 |
| 1,800 mg | Progesterone | 165 | 86 | 12 | 13 |
| (N = 8) | Corticosterone | 455 | 259 | 54 | 109 |

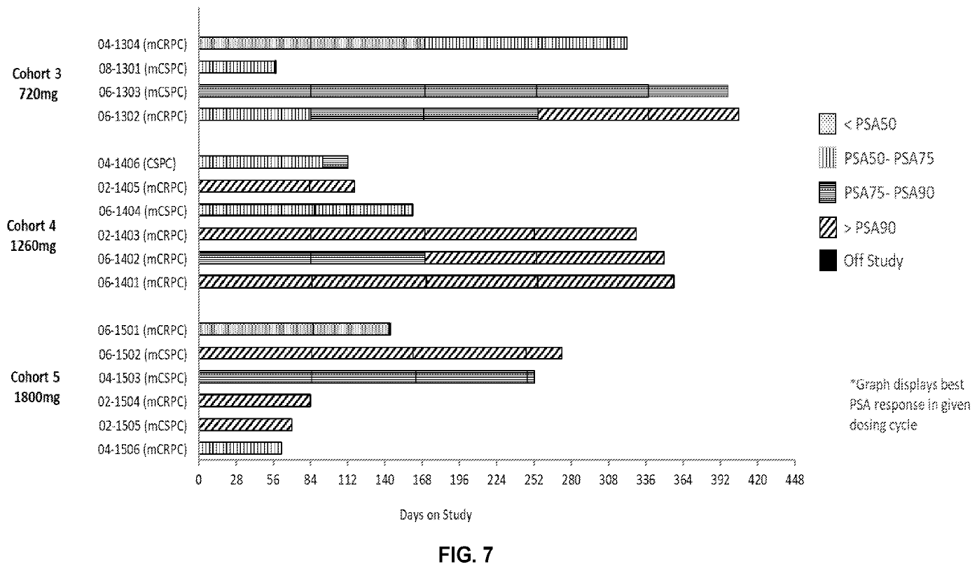

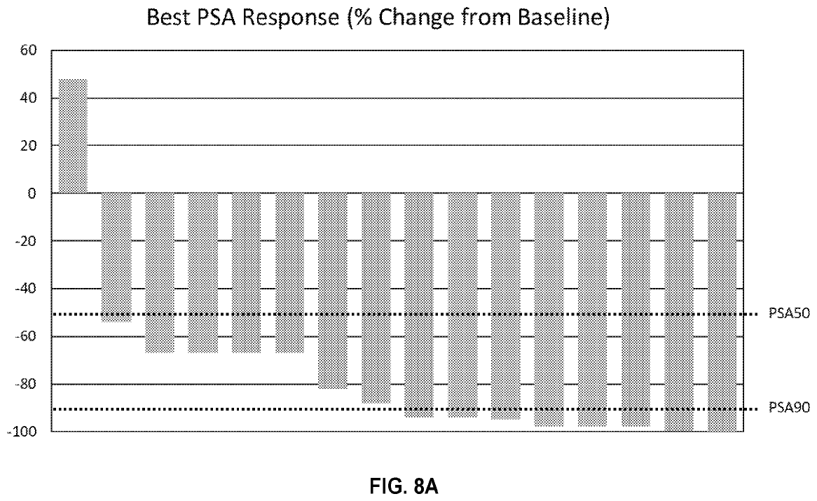

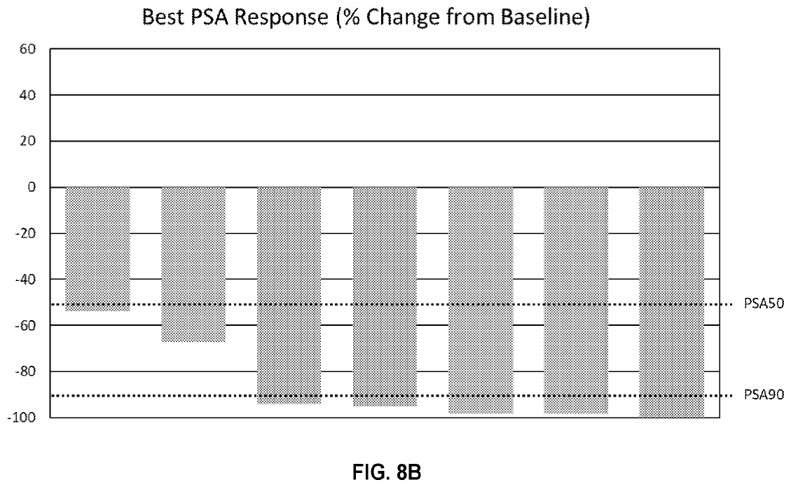

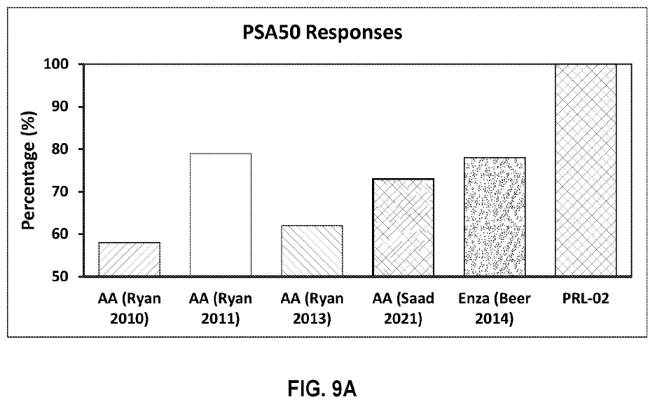

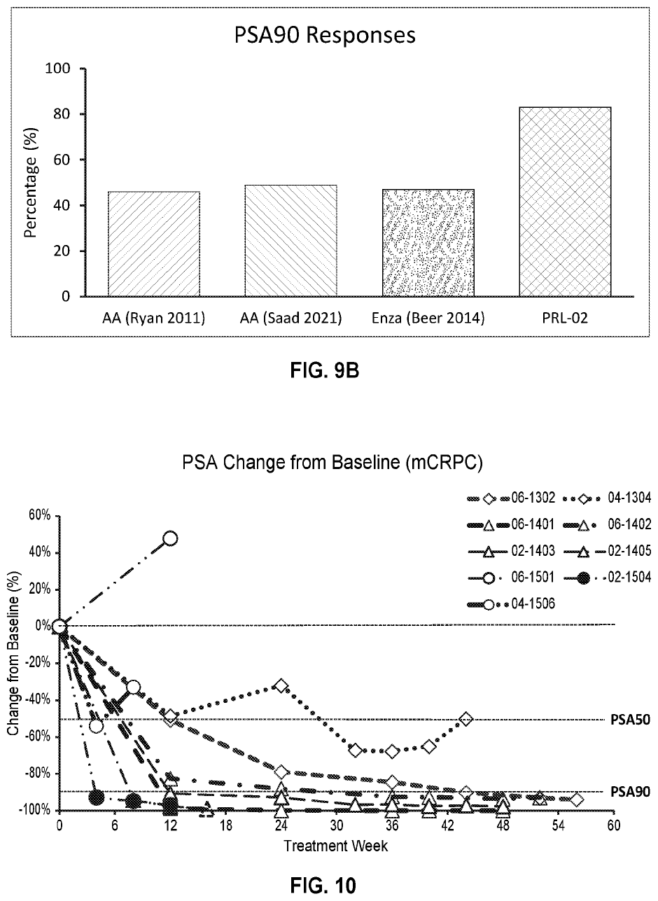

PSA Levels: the median baseline PSA level was 3.01 ng/mL. The partial results show that PSA50 was observed in 15 out of 16 patients (cohorts 3, 4, and 5), see FIGS. 7, 8A, 8B, and 10. PSA90 was observed in 8 out of 16 patients. As shown in FIGS. 9A and 9B, the percentages of patients (Chemo-naïve CRPC) achieved PSA50 and 90 after treatment with PRL-02 (abiraterone decanoate i.m.) herein are much higher than those reported for abiraterone acetate (AA) and enzalutamide (Enza). The table below further summarizes the reduction of PSA observed without elevated progesterone level.

TABLE 3D
Robust PSA Activity Without Elevated Progesterone 100% of Chemo-naïve CRPC
PSA Declines >50%; 75% of CSPC Pts Have Undetectable PSA^{‡}

| | | | | | | | Progesterone | |
|---|---|---|---|---|---|---|---|---|
| | | Dose | | Baseline | Best PSA | | at Visit Day | Undetectable |
| Cohort | ID | (mg) | Group | PSA | Response* | Visit | (nM)\hat{ } | PSA^{#} |
| 5 | 06-1501 | 1800 | mCRPC | 19.18 | 48% | C1D84 | <LLOQ | No |
| 5 | 06-1502 | 1800 | mCSPC | 2.83 | −99.6% | C2D84 | <LLOQ | Yes |
| 5 | 04-1503 | 1800 | mCSPC | 0.03 | −67% | C1D84 | 2.54 | Yes |
| 5 | 02-1504 | 1800 | mCRPC | 10.07 | −92.4 | C1D28 | <LLOQ | No |
| 5 | 02-1505 | 1800 | mCSPC | 21.96 | −98% | C1D84 | 0.50 | No |
| 5 | 04-1506 | 1800 | mCRPC | 0.52 | −54% | C1D28 | <LLOQ | No |
| 4 | 06-1401 | 1260 | mCRPC | 39.54 | −99.97% | C3D84 | <LLOQ | Yes |
| 4 | 06-1402 | 1260 | mCRPC | 10.43 | −93% | C4D28 | <LLOQ | No |
| 4 | 02-1403 | 1260 | mCRPC | 19.45 | −98% | C4D28 | <LLOQ | No |
| 4 | 06-1404 | 1260 | mCSPC | 0.03 | −67% | C2D84 | <LLOQ | Yes |
| 4 | 02-1405 | 1260 | mCRPC | 311.80 | −94% | C1D56 | <LLOQ | No |
| 4 | 04-1406 | 1260 | mCSPC | 3.18 | −82% | C1D56 | 2.90† | No |

^{‡}7/7 CRPC Pts and 3/4 CSPC Pts Results to date
*A post-baseline PSA value of 0.01 ng/mL was used for response calculation if the lab value was << ultra-sensitive assay LLOQ (0.02 ng/mL)
\hat{ }Progesterone assay LLOQ = 1.59 nM; only 2 of 21 samples after Cycle 1 > 2 nM (2.64, 3.34 nM), both at 1800 mg dose
^{#}Defined as PSA < 0.2 ng/mL (clinically undetectable); 5 of 6 PSA values < ultra-sensitive assay LLOQ (0.02 ng/mL)
†Average of Day 28 and Day 70 values.

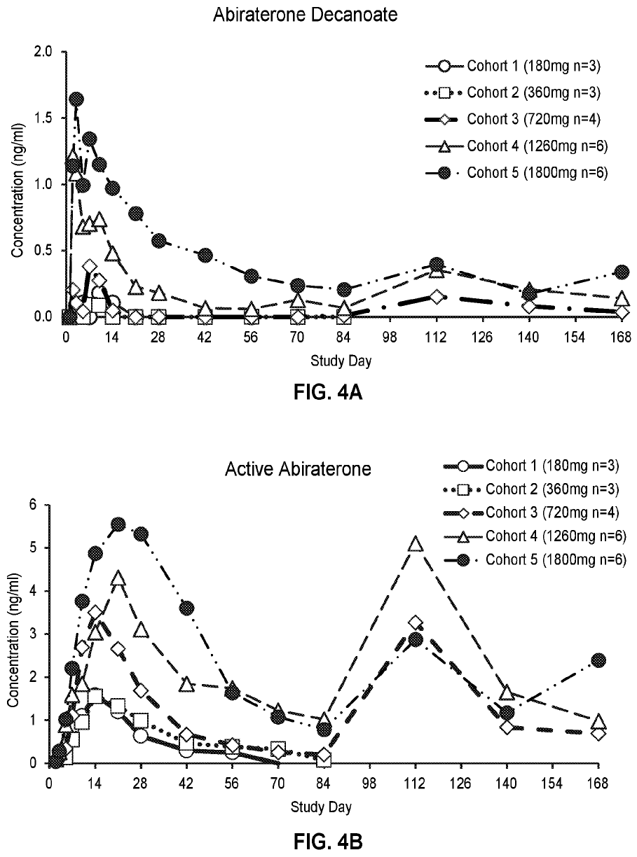

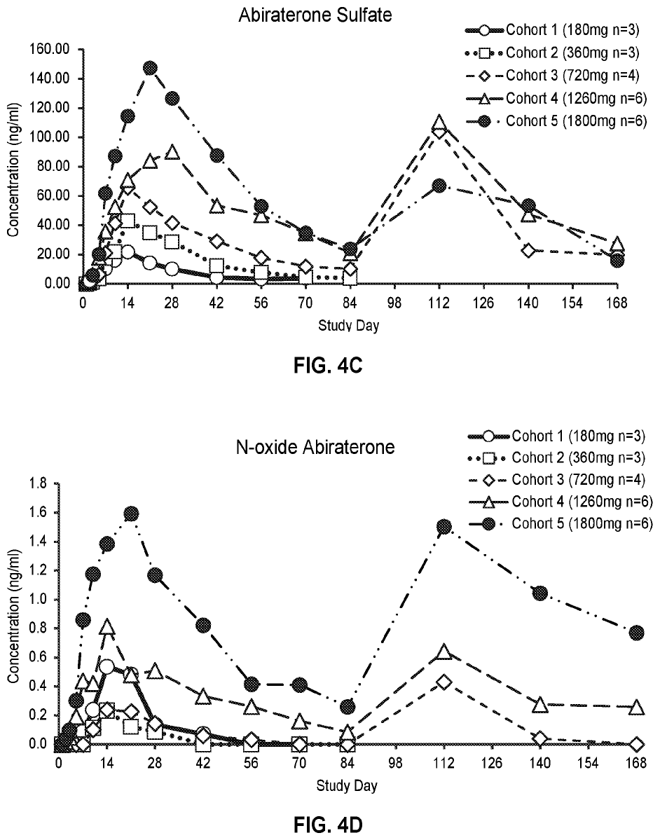

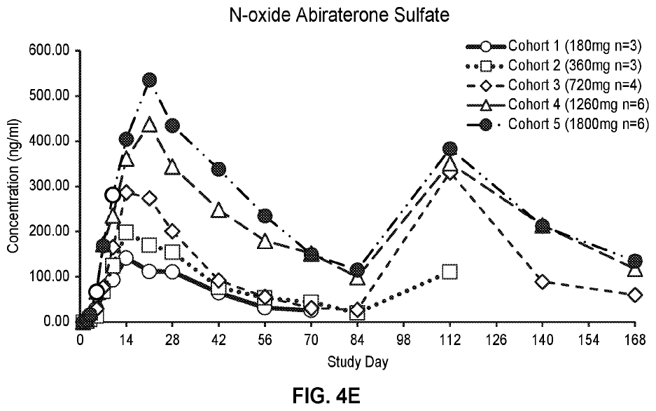

Pharmacokinetic results: the partial results from pharmacokinetic analysis for cohorts 1, 2, 3, 4, and 5 (180 mg, 360 mg, 720 mg, 1260 mg, and 1800 mg abiraterone decanoate, respectively) are shown in FIGS. 4A-4E. It is to be noted that in cohort 3, patient 08-1301 is the only patient across all cohorts to remain below the LLOQ for all abiraterone values. And for abiraterone decanoate, minimal increase in AbiDec for 08-1301 was observed which declined to 0 ng/ml thereafter. Cₘₐₓ observed for the 1800 mg dose (cohort 5) was 5.55 ng/ml during the first cycle (0-84 days), and 2.88 ng/ml during the second cycle. Cₘₐₓ observed for the 1260 mg dose (cohort 4) was 4.32 ng/ml during the first cycle (0-84 days), and 5.11 ng/ml during the second cycle.

Safety results: the partial results also show that the safety profile for all cohorts 1-5 are excellent. Despite small increases in steroids upstream from CYP17A1 hydroxylase, no safety issues relating to mineralocorticoid excess noted to date in Phase 1 study. Increases in upstream steroids from CYP17A1 hydroxylase are also far less than observed with Zytiga.

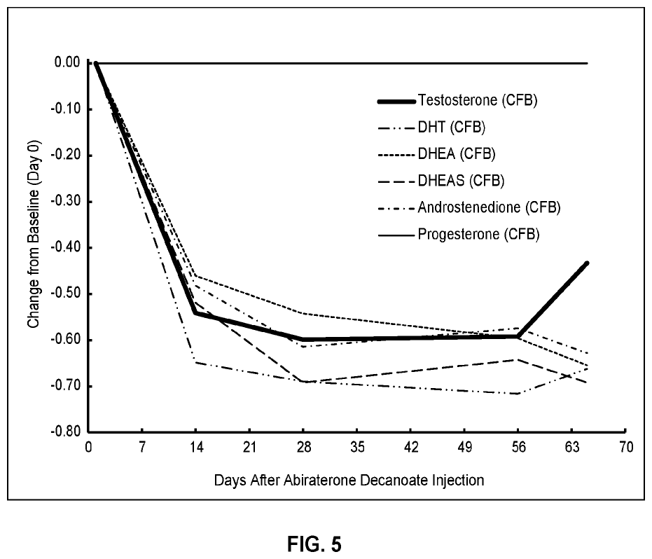

Patient 1. This patient is in cohort 1, which was administered 180 mg of abiraterone decanoate. This patient is 62 year old, with CSPC, with a Gleason score ≤6, who entered the study with left common iliac and femoral lymph node involvement and measurable PSA. The patient received degarelix (March thru May 2021) and leuprolide (starting June 2021). Abiraterone decanoate was injected to the patient on Jul. 12, 2021, although sustained androgen suppression was observed (with 59-61% decline in testosterone from treatment days 21-56), see FIG. 5, the patient did not reach a testosterone level of ≤1 ng/dL and came off the clinical study per clinical protocol. As also shown in FIG. 5, in this patient, no progesterone increase from baseline was observed, which supports that Abiraterone Decanoate preferentially inhibits CYP17A1 Lyase and not CYP17A1 Hydroxylase. On Sep. 24, 2021, this patient started enzalutamide (Xtandi) treatment. Follow-up analysis and post-study PET/CT scan show that this patient achieved complete resolution of prior lymph node lesions, and PSA below LLOQ.

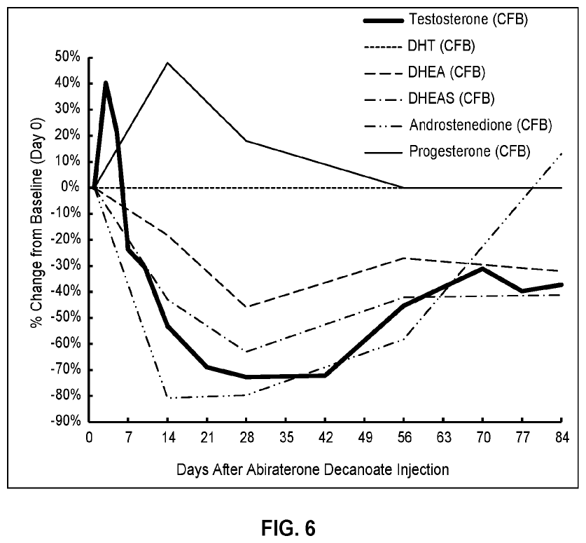

Patient 2. This patient is in cohort 2, which was administered 360 mg of abiraterone decanoate. This patient is a 68-year old male with CSPC, with a Gleason score of 7, who entered the study with sclerotic lesions at the sternum and right iliac bone. The patient received Lupron (2011 thru 2012), radiotherapy (May 2012), Eligard (2015), docetaxel (2015), Provenge (2016), Keytruda (2018), Lupron (2018), and Xtandi (October 2019-June 2021). The steroid response of this patient was shown in FIG. 6. As can be seen from FIG. 6, this patient also achieved sustained androgen suppression. There was a transient ‘up stream’ steroid increase from baseline (progesterone) that returned to baseline suggesting Abiraterone Decanoate preferentially blocking CYP17A1 Lyase.

### Example 4. Preparation of Abiraterone Decanoate Formulations and Stability Tests

This example shows a procedure for preparing various abiraterone decanoate formulations at 180 mg/mL and tests their storage stabilities. Formulation 1 does not contain an added antioxidant. Formulation 1 was filled into vials with or without nitrogen overlay. Formulation 2 contains monothioglycerol as an antioxidant. Formulation 2 was filled into vials with nitrogen overlay. Formulation 3 contains alpha-tocopherol as an antioxidant. Formulation 3 was filled into vials with nitrogen overlay.

The preparation of abiraterone decanoate drug substance is described in WO 2022/174134, published Aug. 18, 2022.

For analysis of drug substance (abiraterone decanoate), assay, related substances, and identification by retention time are conducted using a reversed phase high performance liquid chromatographic analytical method. Separation is performed with an Advanced Materials Technology Halo C8 reversed phase column using dimensions of 3.0×100 mm and a particle size of 2.7 μm. A linear gradient program (20 minutes) is used with mobile phases consisting of a 25 mM aqueous ammonium acetate buffer and a mixture of methanol and acetonitrile. Working standard and sample solutions are prepared in a methanol diluent. The typical injection volume is 5 μL and the detection wavelength is 210 nm.

Gradient Profile:
Mobile Phase A:
25 mM Ammonium Acetate in 90% water, 10% Methanol
Mobile Phase B:
25 mM Ammonium Acetate in 90% acetonitrile, 10%
Methanol

| Time (min) | Mobile Phase A (%) | Mobile Phase B (%) |
|---|---|---|
| 0 | 50 | 50 |
| 20 | 5 | 95 |
| 25 | 5 | 95 |
| 25.1 | 50 | 50 |
| 30 | 50 | 50 |

The details of Formulations 1-3 are shown below. Formulation 1: for each mL, Abiraterone decanoate, 180.0 mg, benzyl alcohol, 100.0 mg, benzyl benzoate, 200.0 mg, corn oil (super refined), q.s. to 1.0 mL. Formulation 2: for each mL, Abiraterone decanoate, 180.0 mg, benzyl alcohol, 100.0 mg, benzyl benzoate, 200.0 mg, monothioglycerol, 2.0 mg, corn oil (super refined), q.s. to 1.0 mL. Formulation 3: for each mL, Abiraterone decanoate, 180.0 mg, benzyl alcohol, 100.0 mg, benzyl benzoate, 200.0 mg, alpha-tocopherol, 0.75 mg, corn oil (super refined), q.s. to 1.0 mL.

The vials used in this study are 10 mL, 20 mm, Clear Glass Crimp Vial, Blowback (West ID 68000390). Stoppers are Datwyler Serum 20 mm, FM259/0 Omniflex Dark Gray Type I (Material Code 110010664). Seals are various 20 mm flip-off aluminum seal.

A brief description of the process for preparing and packaging the formulations 1-3 are shown below. Inspect a 1000 or 500 mL bottle or beaker to be used as a compounding vessel for cleanliness and determine the tare weight (Tare Wt). Add the required amount of benzyl benzoate. Add the required amount of benzyl alcohol. Initiate mixing. Mix until the solution is homogenous. As applicable, add the required amount of monothioglycerol or alpha-tocopherol. Mix until the solution is homogenous. Add the required amount of drug substance to the container. Mix for NLT 30 minutes. Continue mixing until all API is dissolved. If API does not dissolve Corn Oil can be added in the next step to assist in the dissolution. Add Corn Oil (Super Refined) to the solution to assist in the dissolution of the API. If not added note this step as N/A in the Observation section. Q.S. the solution to the target net weight with Corn Oil (Super Refined). Note the new gross weight and net weight of the solution. Mix the solution until it is homogenous. Filter drug product solution through a PVDF 0.2 μm filter into a depyrogenated Pyrex glass vessel with screw cap. Calibrate the filling peristaltic pump based on the filling parameters (fill target, 5.42 g/5.5 mL). Fill 5.5 mL into 10 mL vials and record the net weights of vials. Record the net weight of the first three vials filled then every tenth vial, and finally the last vial. Hand stopper the vials and seal them with a semi-automatic crimper or manual crimper (based on availability). As applicable, for some vials, do not add a nitrogen headspace before stoppering. For other vials, overlay each vial with filtered nitrogen for 10 s before placing the stopper on the vial.

Stability Test: The following table shows test methods and acceptance criteria for prepared batches at initial (t=0) and stability:

| Test | Method | Acceptance Criteria |
|---|---|---|
| Visual Appearance | general | Clear, slightly yellow solution |
| | appearance of the | visibly free of particulates or |
| | material under | foreign matter |
| | test based on | |
| | what an end user | |
| | would observe | |
| | under normal user | |
| | conditions | |
| Particulates (HIAC) | TP00036* | NMT 6000 particles ≥10 μm; |
| | | NMT 600 particles ≥25 μm |
| HPLC Assay/RS | TP77610** | Assay: 90.0-110.0% |
| | | Related Substances: Report |
| | | Results |
| Color of Solution | Ph. Eur. 2.2.2 | Report Results |

*TP00036 is based on USP <788> and USP <789>.
**TP77610 uses the HPLC assay as shown below.

The prepared formulations were also subject to stability test under the following conditions:

| | Vial Configuration-Inverted | |
|---|---|---|
| | Storage Condition | Sample Pull Time Points |
| Initial or t = 0 | 25° C./60% RH | 1, 2, 3 and 9 months |
| | 40° C./75% RH | 1, 2, 3 and 9 months |

For analysis of abiraterone decanoate corn oil solutions, assay, related substances, and identification by retention time are conducted using a reversed phase high performance liquid chromatographic analytical method (TP77610). Separation is performed with an XBridge Shield RP18 reversed phase column using dimensions of 4.6×100 mm and a particle size of 3.5 lm, column temperature, 45° C., flow rate, 1.5 mL/min. A linear gradient program (25 minutes) is used with mobile phases consisting of a 40 mM aqueous ammonium bicarbonate buffer and a mixture of methanol and acetonitrile (ACN). Working standard and sample solutions are prepared in isopropyl alcohol diluent. The typical injection volume is 10 μL and the detection wavelength is 254 nm.

Gradient Profile:
Mobile Phase A:
50:25:25 40 mM Ammonium Bicarbonate Buffer:MeOH:ACN
Mobile Phase B:
50:50 MeOH:ACN

| Time (min) | Mobile Phase A (%) | Mobile Phase B (%) |
|---|---|---|
| 0 | 100 | 0 |
| 25 | 0 | 100 |
| 35 | 0 | 100 |
| 35.1 | 100 | 0 |
| 40 | 100 | 0 |

### Results of Stability Test:

TABLE 4A
Stability of Formulation 1 without nitrogen headspace at 25° C./60% RH Inverted

| | | | Timepoint | | | | |
|---|---|---|---|---|---|---|---|
| | | | 0 Month | 1 Month | 2 Month | 3 Month | 9 Month |
| Test | Method | Acceptance Criteria | Results | Results | Results | Results | Results |
| Appearance | | Report results | Conforms; Clear, | Clear, slightly | Clear, slightly | Clear, slightly | Clear, slightly |
| | | | slightly yellow | yellow solution | yellow solution | yellow solution | yellow solution |
| | | | solution visibly | visibly free of | visibly free of | visibly free of | visibly free of |
| | | | free of | particulates | particulates | particulates | particulates |
| | | | particulates or | or foreign | or foreign | or foreign | or foreign |
| | | | foreign matter | matter | matter | matter | matter |
| Particulates | 00036 | 10 μm: Report results | 7 particles | Not Scheduled | Not Scheduled | 6 particles | Not Scheduled |
| (HIAC) | | 25 μm: Report results | 2 particles | | | 3 particles | |
| HPLC Assay | 77610 | Report results (% LC) | 99.7 | 99.2 | 99.6 | 98.6 | 98.5 |
| HPLC Related | 77610 | Any Individual Impurity: | 1.38/0.10 | 1.39/0.11 | 1.22/0.10 | 0.90/0.10 | 0.90/0.10 |
| Substances | | Report results (RRT/%) | | | | 1.20/0.10 | 1.19 (~1.192)/0.19 |
| | | | | | | 1.22/0.14 | 1.21 (~1.211)/0.33 |
| | | | | | | | 1.24/0.12 |
| | | Total Impurities: | 0.10 | 0.11 | 0.10 | 0.34 | 0.74 |
| | | Report results (%) | | | | | |
| Color of | 79021* | Report results | More intense | EP Color of | EP Color: More | EP Color: More | EP Color: More |
| Solution | | | than Y5, but | Solution: More | intense than | intense than | intense than |
| Ph. Eur. 2.2.2 | | | less intense | intense than | Y4, but less | Y4, but less | Y3, but less |
| | | | than Y4 | Y5, but less | Intense than | intense than | intense than |
| | | | | intense than | Y3. | Y3 | Y2 |
| | | | | Y4 | | | |

*Method 79021 is based on Ph. Eur. 2.2.2.

TABLE 4C
Stability of Formulation 1 with nitrogen headspace at 25° C./60% RH Inverted

| | | | Timepoint | | | | |
|---|---|---|---|---|---|---|---|
| | | | 0 Month | 1 Month | 2 Month | 3 Month | 9 Month |
| Test | Method | Acceptance Criteria | Results | Results | Results | Results | Results |
| Appearance | | Report results | Conforms; Clear, | Clear, slightly | Clear, slightly | Clear, slightly | Clear, slightly |
| | | | slightly yellow | yellow solution | yellow solution | yellow solution | yellow solution |
| | | | solution visibly | visibly free of | visibly free of | visibly free of | visibly free of |
| | | | free of | particulates or | particulates or | particulates or | particulates or |
| | | | particulates or | foreign matter | foreign matter | foreign matter | foreign matter |
| | | | foreign matter | | | | |
| Particulates | 00036 | 10 μm: Report results | 13 particles | Not Scheduled | Not Scheduled | 1 particle | Not Schedules |
| (HIAC) | | 25 μm: Report results | 5 particles | | | 1 particle | |
| HPLC Assay | 77610 | Report results (% LC) | 100.1 | 99.3 | 99.5 | 98.6 | 99.2 |
| HPLC Related | 77610 | Any Individual Impurity: | 1.38/0.10 | 1.39/0.11 | 1.39/0.10 | 1.39/0.10 | 1.19 (~1.192)/0.22 |
| Substances | | Report results | | | | | 1.21 (~1.211)/0.32 |
| | | (RRT/%) Total Impurities: | 0.10 | 0.11 | 0.10 | 0.10 | 0.54 |
| | | Report results (%) | | | | | |
| Color of | 79021 | Report results | More intense | EP Color of | EP Color: More | EP Color: More | EP Color: More |
| Solution | | | than Y5, but | Solution: More | intense than Y5, | intense than Y5, | intense than Y4, |
| Ph. Eur. 2.2.2 | | | less intense | intense than | but less intense | but less intense | but less intense |
| | | | than Y4 | Y6, but less | than Y4. | than Y4 | than Y3 |
| | | | | intense than Y5 | | | |

TABLE 4D
Stability of Formulation 1 with nitrogen headspace at 40° C./75% RH Inverted

| | | | Timepoint | | | |
|---|---|---|---|---|---|---|
| | | | 1 Month | 2 Month | 3 Month | 9 Month |
| Test | Method | Acceptance Criteria | Results | Results | Results | Results |
| Appearance | | Report results | Clear, slightly | Clear, slightly | Clear, slightly | Clear, slightly |
| | | | yellow solution | yellow solution | yellow solution | yellow solution |
| | | | visibly free of | visibly free of | visibly free of | visibly free of |
| | | | particulates or | particulates or | particulates or | particulates or |
| | | | foreign matter | foreign matter | foreign matter | foreign matter |
| Particulates | 00036 | 10 μm: Report results | Not Scheduled | Not Scheduled | 2 particles | Not Scheduled |
| (HIAC) | | 25 μm: Report results | | | 1 particle | |
| HPLC Assay | 77610 | Report results (% LC) | 99.4 | 99.7 | 99.2 | 99.7 |
| HPLC Related | 77610 | Any Individual Impurity: | 1.19/0.13 | 1.22/0.11 | 1.22 (1.215)/0.10 | 1.19 (~1.187)/0.13 |
| Substances | | Report results (RRT/%) | 1.22/0.11 | | 1.22 (1.218)/0.12 | 1.19 (~1.192)/0.15 |
| | | | 1.39/0.12 | | | 1.21 (~1.211)/0.17 |
| | | | | | | 1.21 (~1.214)/0.18 |
| | | Total Impurities: Report | 0.36 | 0.11 | 0.21 | 0.62 |
| | | results (%) | | | | |
| Color of | 79021 | Report results | EP Color of | EP Color: More | EP Color: Y3 | EP Color: More |
| Solution | | | Solution: More | intense than | | intense than Y2, |
| Ph. Eur. 2.2.2 | | | intense than Y4, | Y4, but less | | but less intense |
| | | | but less intense | intense than Y3. | | than Y1. |
| | | | than Y3 | | | |

TABLE 4E
Stability of Formulation 2 with nitrogen headspace at 25° C./60% RH Inverted

| | | | Timepoint | | | | |
|---|---|---|---|---|---|---|---|
| | | | 0 Month | 1 Month | 2 Month | 3 Month | 9 Month |
| Test | Method | Acceptance Criteria | Results | Results | Results | Results | Results |
| Appearance | | Report results | Conforms; Clear, | Clear, slightly | Clear, slightly | Clear, slightly | Clear, slightly |
| | | | slightly yellow | yellow solution | yellow solution | yellow solution | yellow solution |
| | | | solution visibly | visibly free of | visibly free of | visibly free of | visibly free of |
| | | | free of | particulates | particulates | particulates | particulates |
| | | | particulates or | or foreign | or foreign | or foreign | or foreign |
| | | | foreign matter | matter | matter | matter | matter |
| Particulates | 00036 | 10 μm: Report results | 13 particles | Not Scheduled | Not Scheduled | 2 particles | Not Scheduled |
| (HIAC) | | 25 μm: Report results | 4 particles | | | 1 particle | |
| HPLC Assay | 77610 | Report results (% LC) | 100.3 | 99.6 | 99.5 | 99.4 | 100.2 |
| HPLC Related | 77610 | Any Individual Impurity: | 1.38/0.10 | 1.39/0.11 | 1.39/0.10 | 1.39/0.10 | <0.10 |
| Substances | | Report results | | | | | |
| | | (RRT/%) Total Impurities: | 0.10 | 0.11 | 0.10 | 0.10 | <0.10 |
| | | Report results (%) | | | | | |
| Color of | 79021 | Report results | More intense | EP Color of | EP Color: More | EP Color: More | EP Color: |
| Solution | | | than Y5, but | Solution: More | intense than | intense than | More intense |
| Ph. Eur. | | | less intense | intense than | Y6, but less | Y6, but less | than Y5, but |
| 2.2.2 | | | than Y4 | Y6, but less | intense than | intense than | less intense |
| | | | | intense than Y5 | Y5. | Y5 | than Y4 |

TABLE 4F
Stability of Formulation 2 with nitrogen headspace at 40° C./75% RH Inverted

| | | | Timepoint | | | |
|---|---|---|---|---|---|---|
| | | | 1 Month | 2 Month | 3 Month | 9 Month |
| Test | Method | Acceptance Criteria | Results | Results | Results | Results |
| Appearance | | Report results | Clear, slightly | Clear, slightly | Clear, slightly | Clear, slightly |
| | | | yellow solution | yellow solution | yellow solution | yellow solution |
| | | | visibly free of | visibly free of | visibly free of | visibly free of |
| | | | particulates or | particulates or | particulates or | particulates or |
| | | | foreign matter | foreign matter | foreign matter | foreign matter |
| Particulates (HIAC) | 00036 | 10 μm: Report results | Not Scheduled | Not Scheduled | 3 particles | Not Scheduled |
| | | 25 μm: Report results | | | 2 particles | |
| HPLC Assay | 77610 | Report results (% LC) | 100.2 | 100.2 | 98.9 | 100.5 |
| HPLC Related | 77610 | Any Individual Impurity: | 1.39/0.11 | Unknown | Unknown | <0.10 |
| Substances | | Report results | | Impurities <0.10 | Impurity <0.10 | |
| | | (RRT/%) Total Impurities: | 0.11 | <0.10 | <0.10 | <0.01 |
| | | Report results (%) | | | | |
| Color of | 79021 | Report results | EP Color of | EP Color: More | EP Color: More | EP Color: More |
| Solution | | | Solution: More | intense than | intense than | intense than |
| Ph. Eur. 2.2.2 | | | intense than | Y6, but less | Y6, but less | Y5, but less |
| | | | Y6, but less | intense than | intense than | intense than |
| | | | intense than Y5 | Y5. | Y5 | Y4 |

TABLE 4G
Stability of Formulation 3 with nitrogen headspace at 25° C./60% RH Inverted

| | | | Timepoint | | | | |
|---|---|---|---|---|---|---|---|
| | | | 0 Month | 1 Month | 2 Month | 3 Month | 9 Month |
| Test | Method | Acceptance Criteria | Results | Results | Results | Results | Results |
| Appearance | | Report results | Conforms; Clear, | Clear, slightly | Clear, slightly | Clear, slightly | Clear, slightly |
| | | | slightly yellow | yellow solution | yellow solution | yellow solution | yellow solution |
| | | | solution visibly | with a | visibly free of | visibly free of | visibly free of |
| | | | free of | black fiber | particulates or | particulates or | particulates or |
| | | | particulates or | | foreign matter | foreign matter | foreign matter |
| | | | foreign matter | | | | |
| Particulates | 00036 | 10 μm: Report results | 9 particles | Not Scheduled | Not Scheduled | 1 particle | Not Scheduled |
| (HIAC) | | 25 μm: Report results | 2 particles | | | 1 particle | |
| HPLC Assay | 77610 | Report results (% LC) | 99.7 | 99.3 | 99.2 | 98.9 | 99.5 |
| HPLC Related | 77610 | Any Individual Impurity: | 1.38/0.10 | 1.39/0.11 | 1.39/0.10 | 1.20/0.13 | 1.19 (~1.192)/ |
| Substances | | Report results (RRT/%) | | | | 1.22/0.12 | 0.28 |
| | | | | | | 1.39/0.10 | 1.2 (~1.211)/ |
| | | | | | | | 0.32 |
| | | Total Impurities: | 0.10 | 0.11 | 0.10 | 0.34 | 0.60 |
| | | Report results (%) | | | | | |
| Color of | 79021 | Report results | More intense | EP Color of | EP Color: More | EP Color: More | EP Color: More |
| Solution | | | than Y5, but | Solution: | intense than | intense than | intense than |
| Ph. Eur. 2.2.2 | | | less intense | More intense | Y6, but less | Y5, but less | Y4, but less |
| | | | than Y4 | than Y6, but | intense than | intense than | intense than |
| | | | | less intense | Y5. | Y4 | Y3 |
| | | | | than Y5 | | | |

TABLE 4H
Stability of Formulation 3 with nitrogen headspace at 40° C./75% RH Inverted

| | | | Timepoint | | | |
|---|---|---|---|---|---|---|
| | | | 1 Month | 2 Month | 3 Month | 9 Month |
| Test | Method | Acceptance Criteria | Results | Results | Results | Results |
| Appearance | | Report results | Clear, slightly | Clear, slightly | Clear, slightly | Clear, slightly |
| | | | yellow solution | yellow solution | yellow solution | yellow solution |
| | | | visibly free of | visibly free of | visibly free of | visibly free of |
| | | | particulates or | particulates or | particulates or | particulates or |
| | | | foreign matter | foreign matter | foreign matter | foreign matter |
| Particulates | 00036 | 10 μm: Report results | Not Scheduled | Not Scheduled | 3 particles | Not Scheduled |
| (HIAC) | | 25 μm: Report results | | | 1 particle | |
| HPLC Assay | 77610 | Report results (% LC) | 98.8 | 99.4 | 98.9 | 99.3 |
| HPLC Related | 77610 | Any Individual Impurity: | 1.19/0.17 | 1.22/0.10 | 1.22/0.12 | 1.19 (~1.192)/0.17 |
| Substances | | Report results (RRT/%) | 1.22/0.14 | 1.39/0.10 | 1.39/0.10 | 1.21 (~1.211)/0.14 |
| | | | 1.39/0.12 | | | 1.21 (~1.214)/0.15 |
| | | Total Impurities: Report | 0.44 | 0.20 | 0.22 | 0.45 |
| | | results (%) | | | | |
| Color of | 79021 | Report results | EP Color of | EP Color: More | EP Color: More | EP Color: More |
| Solution | | | Solution: More | intense than Y4, | intense than Y4, | intense than Y3, |
| Ph. Eur. 2.2.2 | | | intense than Y5, | but less intense | but less intense | but less intense |
| | | | but less intense | than Y3. | than Y3 | than Y2 |
| | | | than Y4 | | | |

Conclusion: As shown above, the use of monothioglycerol as an antioxidant increases stability of abiraterone decanoate, with the least color changes and the least amount of total impurities upon storage at 25° C./60% RH or 40° C./75% RH for up to 3 months. This represents a significant improvement over prior abiraterone decanoate formulations which is best stored at 5° C.

### Example 5. Manufacture of Abiraterone Decanoate Solution for Injection

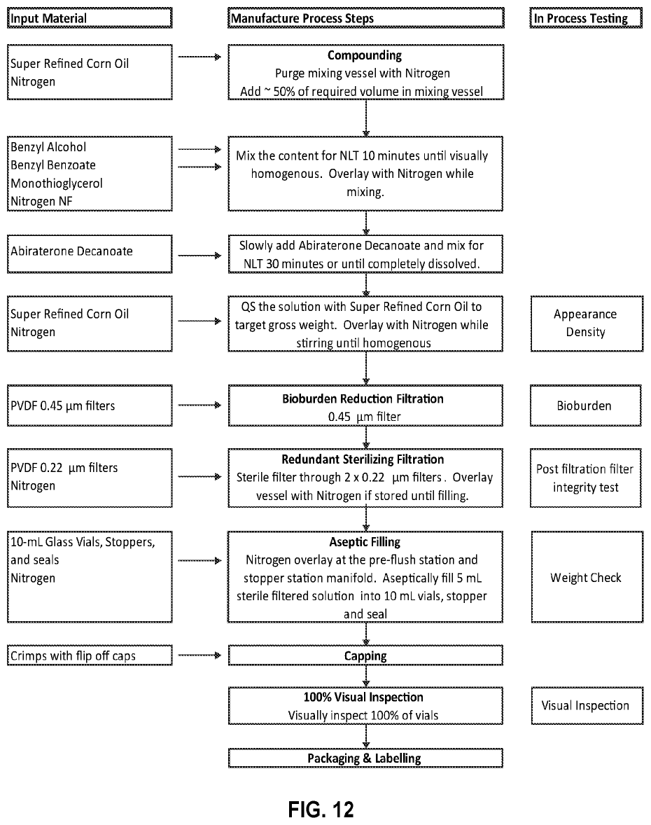

A flow diagram illustrating the steps typically performed in the manufacturing process for Abiraterone Decanoate Solution for IM Injection, 180 mg/mL is provided in FIG. 12. A brief description of the steps in the process is shown below.

Compounding: The mixing vessel is purged with Nitrogen NF prior to adding excipients. Compounding of abiraterone decanoate solution started with approximately 50% of the super refined corn oil required in a mixing vessel. Benzyl alcohol, benzyl benzoate and monothioglycerol are added and mixed until visually homogenous. Nitrogen overlay is added while mixing benzyl alcohol, benzyl benzoate and monothioglycerol. Abiraterone decanoate is then added in portions and mixed until completely dissolved. Super refined corn oil is added to target weight. Nitrogen overlay is added to the bulk solution while stirring until homogenous.

Filtration: The bulk solution is filtered through a bioburden reduction filter. Sample for bioburden is taken after the bioburden filter.

The bioburden reduced bulk solution is filtered through two redundant sterilizing filters in series to provide sterile bulk solution. Post filter integrity test on both sterilizing filters is performed.

Aseptic Filling: Nitrogen overlay is introduced at the pre-flush station and at the stopper station manifold. Aseptically fill 5 mL of the sterile filtered solution into 10 mL vials, stopper, and seal the vials. The target fill volume is 5.5 mL (per guidance in USP <1151> for removable volume) with target weight range=5.42 g+/−3% or 5.26 g-5.58 g. The weights of the vials are checked at the beginning, representative vial per tray in the middle of the fill, and at the end of the fill.

Capping & Visual Inspection: The vials are capped and then 100% visually inspected. Acceptable vials are bulk packed and transfer to packaging and labelling operations.

Each reference referred to within this disclosure is hereby incorporated in its respective entirety.

With respect to aspects of the disclosure described as a genus, all individual species are individually considered separate aspects of the disclosure. If aspects of the disclosure are described as “comprising” a feature, embodiments also are contemplated “consisting of” or “consisting essentially of” the feature.

All the various aspects, embodiments, and options described herein can be combined in any and all variations.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, because numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method of treating a disease or disorder in a human subject in need thereof, comprising parenterally administering to the human subject a therapeutically effective amount of a pharmaceutical composition comprising an abiraterone prodrug (e.g., an abiraterone lipophilic ester), wherein the disease or disorder is sex hormone-dependent or androgen receptor driven, such as a sex hormone-dependent benign or malignant disorder or a syndrome due to androgen excess.

2. The method of claim 1, wherein the abiraterone prodrug is abiraterone decanoate or a pharmaceutically acceptable salt thereof,

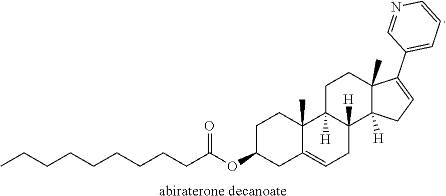

3. The method of claim 1 or 2, wherein the method selectively inhibits CYP17A1 lyase activity over CYP17A1 hydroxylase activity in the human subject.

4. The method of any of claims 1-3, wherein the disease or disorder is polycystic ovary syndrome (PCOS), congenital adrenal hyperplasia (CAH), or endometriosis.

5. The method of any of claims 1-3, wherein the disease or disorder is a cancer, wherein the cancer is a sex hormone dependent or androgen receptor driven cancer.

6. The method of claim 5, wherein the cancer is prostate cancer, endometrial cancer, or ovarian cancer.

7. The method of claim 5, wherein the cancer is prostate cancer.

8. The method of any of claims 5-7, wherein the cancer has metastasized to one or more lymph nodes, and the administering of the pharmaceutical composition is effective in inhibiting growth of the cancer in the one or more lymph nodes.

9. The method of any of claims 5-8, wherein the cancer is a prostate cancer characterized with a Gleason score of ≤6.

10. The method of any of claims 5-9, wherein the human subject has a measurable prostate specific antigen.

11. The method of any of claims 5-7, wherein the cancer is a localized prostate cancer, e.g., a high risk localized prostate cancer.

12. The method of any of claims 5-7, wherein the cancer is a metastatic castration-sensitive prostate cancer, non-metastatic castration-sensitive prostate cancer, non-metastatic castration-resistant prostate cancer, or metastatic castration-resistant prostate cancer.

13. The method of any of claims 5-7, wherein the cancer is a newly diagnosed high risk metastatic hormone sensitive prostate cancer.

14. The method of any of claims 5-7, wherein the cancer is a metastatic CRPC (mCRPC), wherein the human subject is asymptomatic or mildly symptomatic after failure of androgen deprivation therapy in whom chemotherapy is not yet clinically indicated.

15. The method of any of claims 5-7, wherein the cancer is a metastatic CRPC (mCRPC), wherein the human subject's disease has progressed on or after a taxane-based chemotherapy regimen, such as docetaxel-based or cabazitaxel-based chemotherapy regimen.

16. The method of any of claims 5-7, wherein the cancer is a refractory prostate cancer.

17. The method of any one of claims 5-16, wherein (i) the human subject's disease has progressed on or after an androgen receptor antagonist based treatment, such as enzalutamide based treatment; and/or (ii) the human subject has developed resistance to the treatment of abiraterone acetate in combination with prednisone, including resistance due to increased levels of progesterone.

18. The method of any one of claims 1-17, wherein the pharmaceutical composition comprises abiraterone decanoate in its basic form and a pharmaceutically acceptable carrier.

19. The method of claim 18, wherein the pharmaceutically acceptable carrier comprises a pharmaceutically acceptable oil and optionally a further pharmaceutically acceptable solvent.

20. The method of claim 19, wherein the pharmaceutically acceptable oil comprises a triglyceride (e.g., long and/or medium chain triglycerides), and the further pharmaceutically acceptable solvent, if present, comprises an alcohol, ester, and/or acid solvent.

21. The method of claim 19 or 20, wherein the pharmaceutically acceptable oil is selected from vegetable oil, castor oil, corn oil, sesame oil, cottonseed oil, peanut oil, poppy seed oil, tea seed oil, and soybean oil, and the further pharmaceutically acceptable solvent, if present, comprises benzyl alcohol, benzyl benzoate, or a combination thereof.

22. The method of any one of claims 18-21, wherein the pharmaceutically acceptable carrier comprises corn oil, benzyl alcohol, and benzyl benzoate.

23. The method of any one of claims 1-22, wherein the pharmaceutical composition comprises, for each milliliter, (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); and (d) corn oil, q.s. to 1 milliliter.

24. The method of any one of claims 1-22, wherein the pharmaceutical composition comprises, for each milliliter, (a) abiraterone decanoate in its basic form, in an amount of about 180 mg; (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); and (d) corn oil, q.s. to 1 milliliter.

25. The method of any one of claims 18-24, wherein the weight ratio of benzyl alcohol to benzyl benzoate in the pharmaceutical composition ranges from about 2:1 to about 1:5 (e.g., about 1:1 to 1:3, such as about 1:2).

26. The method of any one of claims 1-25, wherein the abiraterone prodrug is abiraterone decanoate, and the abiraterone decanoate is substantially pure, e.g., characterized as having a purity by weight of at least 95%, preferably, at least 98%, such as about 98.5%, about 99%, about 99.5%, or higher.

27. The method of any one of claims 1-26, wherein the pharmaceutical composition is characterized as having a viscosity of less than 0.1 Pa*s, such as about 0.05 Pa*s or lower.

28. The method of any one of claims 1-27, wherein the pharmaceutical composition is characterized as having a glide force of about 1-10 N when measured using a 21G, 1.5 inch needle, and/or about 2-15 N when measured using a 23G, 1.5 inch needle, and/or about 30-150 N when measured using a 27G, 1.5 inch needle.

29. The method of any one of claims 1-28, wherein the pharmaceutical composition is characterized as having no more than 1000 particles having a size of 10 m or greater, and no more than 300 particles having a size of 25 m or greater, when measured according to USP <788> and/or <789>.

30. The method of any one of claims 1-29, wherein the pharmaceutical composition is characterized as having less than 100 EU/ml, such as less than 25 EU/ml of bacterial endotoxins measured according to USP <85>.

31. The method of any one of claims 1-30, wherein the human subject is non-castrated.

32. The method of any one of claims 1-30, wherein the human subject is castrated.

33. The method of any one of claims 1-30 and 32, wherein the human subject is treated with a gonadotropin-releasing hormone agonist and/or antagonist.

34. The method of any one of claims 1-33, wherein the pharmaceutical composition is administered to the human subject through an intramuscular injection.

35. The method of any one of claims 1-34, wherein the pharmaceutical composition is administered to the human subject once every 1-3 months, such as once every three months.

36. The method of any one of claims 1-35, wherein each administration of the pharmaceutical composition comprises administering to the human subject about 50 mg to about 2000 mg of abiraterone decanoate, such as about 180 mg, about 360 mg, about 720 mg, about 1260 mg, about 1800 mg, or any range between the recited values, preferably, the pharmaceutical composition is administered to the human subject once every three months and each administration of the pharmaceutical composition comprises administering to the human subject about 1260 mg of abiraterone decanoate.

37. The method of any one of claims 1-36, further comprising administering to the human subject a 1^{st}-generation androgen receptor antagonist, e.g., proxalutamide, bicalutamide, flutamide, nilutamide, topilutamide.

38. The method of any one of claims 1-37, further comprising administering to the human subject a 2^{nd}-generation androgen receptor antagonist (e.g., apalutamide, darolutamide or enzalutamide).

39. The method of claim 38, wherein the 2ⁿd-generation androgen receptor antagonist is enzalutamide.

40. The method of any one of claims 1-39, further comprising administering to the human subject a 3^{rd} generation androgen receptor antagonist (such as an N-terminal domain inhibitor) or an androgen receptor degrader molecule, alone or in combination with one or more 1^{st} generation or 2^{nd} generation androgen receptor antagonists.

41. The method of any one of claims 1-40, further comprising administering to the human subject one or more agents selected from hydrocortisone, prednisone, prednisolone, methylprednisolone, and dexamethasone, preferably, dexamethasone.

42. The method of any one of claims 1-41, further comprising administering to the human subject a poly ADP ribose polymerase (PARP) inhibitor, e.g., niraparib, rucaparib, olaparib, talazoparib, veliparib, and fluzoparib.

43. The method of any one of claims 1-42, further comprising administering to the human subject a chemotherapeutic agent, such as a taxane based chemotherapeutic agent (e.g., docetaxel, cabazitaxel, paclitaxel, etc.) or platinum based chemotherapeutic agent (e.g., cisplatin, carboplatin, oxaliplatin, etc.).

44. The method of any one of claims 1-43, further comprising administering to the human subject an immunotherapy, such as administering Sipuleucel-T, an immune checkpoint inhibitor (e.g., anti-PD-1 antibody such as pembrolizumab or nivolumab, or anti-PD-L1 antibody such as avelumab or atezolizumab), or an anti-CTLA-4 antibody (e.g., ipilimumab), etc.

45. The method of any one of claims 1-44, further comprising administering to the human subject a bispecific T-cell engager (BiTE) therapy, such as blinatumomab or solitomab.

46. The method of any one of claims 1-45, further comprising administering to the human subject a kinase inhibitor, e.g., sunitinib, dasatinib, cabozantinib, erdafitinib, dovitinib, capivasertib, onvansertib, ipatasertib, afuresertib, alisertib, apitolisib, opaganib, etc.

47. The method of any one of claims 1-46, further comprising administering to the human subject a bone protecting agent (e.g., denosumab, zolendronic acid), and wherein the human subject is characterized as having prostate cancer (e.g., CRPC) with bone metastasis.

48. The method of any one of claims 1-47, further comprising administering to the human subject a therapeutic agent selected from 1) an anti-IL23 targeting monoclonal antibody, e.g., tildrakizumab; 2) a selenium, such as sodium selenite; 3) an EZH2 inhibitor, e.g., CPI-1205, GSK2816126, or tazemetostat; 4) a CDK4/6 inhibitor, e.g., palbociclib, ribociclib, abemaciclib; 6) a bromodomain and extra-terminal domain (BET) inhibitor, e.g., CCS1477, INCB057643, alobresib, ZEN-3694, or molibresib (GSK525762); 7) an anti-CD105 antibody, e.g., TRC105 or carotuximab; 8) niclosamide; 9) an A2A receptor antagonist, e.g., AZD4635; 10) a PI3K inhibitor, e.g., AZD-8186, buparlisib, or dactolisib; 11) a further non-steroidal CYP17A1 inhibitor, e.g. seviteronel; 12) an antiprogestogen, e.g., onapristone; 13) navitoclax; 14) an HSP90 inhibitor, e.g., onalespib (AT13387); 15) an HSP27 inhibitor, e.g., OGX-427; 16) a 5-alpha-reductase inhibitor, e.g., dutasteride; 17) metformin; 18) AMG-386; 19) dextromethorphan; 20) theophylline; 21) hydroxychloroquine; and 22) lenalidomide.

49. The method of any one of claims 1-48, further comprising administering to the human subject one or more kinase modulators selected from FLT-3 (FMS-like tyrosine kinase) inhibitors, AXL (anexelekto) inhibitors (e.g., Gilteritinib), CDK (cyclin dependent kinase) inhibitors, such as CDK1, 2, 4, 5, 6, 7, or 9 inhibitors, retinoblastoma (Rb) inhibitors, protein kinase B (AKT) inhibitors, SRC inhibitors, IkappaB kinase 1 (IKK1) inhibitors, PIM-1 modulators, Lemur tyrosine kinase 2 (LMTK2) modulators, Lyn inhibitors, Aurora A inhibitors, ANPK (a nuclear protein kinase) inhibitors, extracellular-signal regulated kinase (ERK) modulators, c-jun N-terminal kinase (JNK) modulators, Big MAP kinase (BMK) modulators, p38 mitogen-activated protein kinases (MAPK) modulators, and combinations thereof.

50. The method of any one of claims 1-49, wherein the human subject is (i) chemotherapy naïve or hormone therapy naïve prior to being administered the pharmaceutical composition, such as a chemotherapy naïve mCRPC patient; and/or (ii) wherein the human subject suffers from hepatic impairment, such as moderate to severe hepatic impairment (Child-Pugh Class B or C), prior to being administered the pharmaceutical composition).

51. The method of any one of claims 1-50, wherein (i) the human subject has prostate cancer, and the method does not increase the level of progesterone in the human subject to a level associated with poor clinical outcomes and drug resistance, such as a serum or plasma progesterone greater than about 3 nM when measured at 4 weeks, 6 weeks, or 12 weeks after the first administration of the pharmaceutical composition; and/or (ii) the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition.

52. The method of any one of claims 1-51, wherein the human subject suffers from one or more side effects associated with inhibition of CYP17A1 hydroxylase activity or the human subject is susceptible to one or more side effects associated with inhibition of CYP17A1 hydroxylase activity, such as mineralcorticoid toxicity.

53. The method of any one of claims 1-52, wherein the abiraterone prodrug is abiraterone decanoate, and at least a portion of the administered abiraterone decanoate is absorbed through the lymphatic system.

54. The method of any one of claims 1-53, wherein the administering of the pharmaceutical composition is effective in achieving a sustained reduction of serum testosterone level in the human subject to 50% below baseline or lower within 15 days of the first administration of the pharmaceutical composition.

55. The method of claim 54, wherein the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition.

56. The method of any one of claims 1-55, wherein the administering of the pharmaceutical composition does not enhance serum progesterone level in the human subject (i) by more than 40% above baseline at 4 weeks following the first administration of the pharmaceutical composition; and/or (ii) by more than 40% above baseline from 2 weeks to 12 weeks following the second administration of the pharmaceutical composition.

57. The method of any one of claims 1-55, wherein the administering of the pharmaceutical composition does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the pharmaceutical composition.

58. The method of any one of claims 1-57, wherein as applicable, the administering of the pharmaceutical composition reduces the level of prostate specific antigen in the human subject, preferably, the level of prostate specific antigen is reduced to 50% or below, preferably, 90% or below, compared to the baseline PSA level, at least at one time point following the first administration of the pharmaceutical composition and/or following one or more subsequent administration of the pharmaceutical composition.

59. A method of reducing serum testosterone level in a human subject in need thereof, the method comprising parenterally administering to the human subject an effective amount of a pharmaceutical composition comprising an abiraterone prodrug (e.g., an abiraterone lipophilic ester), wherein the pharmaceutical composition is administered in an effective amount to achieve a sustained reduction of serum testosterone level in the human subject to 50% below baseline or lower within 15 days of the first administration of the pharmaceutical composition, wherein the administering of the pharmaceutical composition does not enhance serum progesterone level in the human subject (i) by more than 40% above baseline at 4 weeks following the first administration of the pharmaceutical composition; and/or (ii) by more than 40% above baseline from 2 weeks to 12 weeks following the second administration of the pharmaceutical composition, preferably, does not enhance serum progesterone level in the human subject (i) by more than 20% above baseline at 4 weeks following the first administration of the pharmaceutical composition, (ii) by more than 20% above baseline at 6 weeks, 8 weeks, 10 weeks, and/or 12 weeks following the first administration of the pharmaceutical composition; and/or (iii) by more than 20% above baseline from 2 weeks to 12 weeks following the second administration of the pharmaceutical composition.

60. The method of claim 59, wherein the abiraterone prodrug is abiraterone decanoate, or a pharmaceutically acceptable salt thereof,

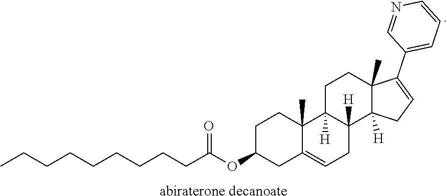

61. The method of claim 59 or 60, wherein the human subject suffers from a disease or disorder that is sex hormone-dependent or androgen receptor driven, such as a sex hormone-dependent benign or malignant disorder or a syndrome due to androgen excess.

62. The method of claim 61, wherein the disease or disorder is any of those described in claims 4-17.

63. The method of any of claims 59-62, wherein the sustained reduction of serum testosterone level is characterized in that once the serum testosterone level in the human subject is reduced to 50% below baseline or lower, the serum testosterone level remains at 50% below baseline or lower up to 8 weeks or longer following the first administration of the pharmaceutical composition.

64. The method of any of claims 59-63, wherein the human subject suffers from one or more side effects associated with inhibition of CYP17A1 hydroxylase activity or the human subject is susceptible to one or more side effects associated with inhibition of CYP17A1 hydroxylase activity, such as mineralcorticoid toxicity.

65. A method of treating a cancer in a human subject in need thereof, wherein the cancer is a sex hormone dependent or androgen receptor driven cancer, which has metastasized to one or more lymph nodes, the method comprising parenterally administering to the human subject a therapeutically effective amount of a pharmaceutical composition comprising an abiraterone prodrug (e.g., an abiraterone lipophilic ester).

66. The method of claim 65, wherein the abiraterone prodrug is abiraterone decanoate, or a pharmaceutically acceptable salt thereof,

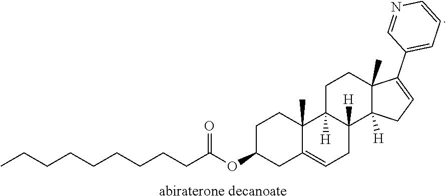

67. The method of claim 65 or 66, wherein the administering of the pharmaceutical composition is effective in inhibiting growth of the cancer in the one or more lymph nodes.

68. The method of any of claims 65-67, wherein the cancer is prostate cancer.

69. A method of treating a cancer in a human subject in need thereof, wherein the cancer is a sex hormone dependent or androgen receptor driven cancer, wherein the human subject's disease has progressed on or after an androgen receptor antagonist based treatment, such as enzalutamide based treatment, the method comprising parenterally administering to the human subject a therapeutically effective amount of a pharmaceutical composition comprising an abiraterone prodrug (e.g., an abiraterone lipophilic ester).

70. The method of claim 69, wherein the abiraterone prodrug is abiraterone decanoate, or a pharmaceutically acceptable salt thereof,

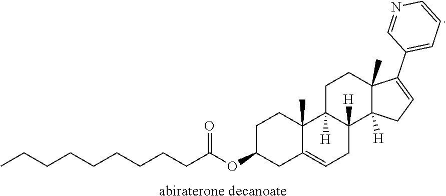

71. The method of claim 69 or 70, wherein the cancer is prostate cancer.

72. A method of treating a cancer in a human subject in need thereof, wherein the cancer is a sex hormone dependent or androgen receptor driven cancer, the method comprising parenterally administering to the human subject a therapeutically effective amount of a pharmaceutical composition comprising an abiraterone prodrug (e.g., an abiraterone lipophilic ester), wherein the human subject is further treated with an androgen receptor antagonist, such as enzalutamide, prior to, concurrent, or subsequent to the first parenteral administration of the pharmaceutical composition.

73. The method of claim 72, wherein the abiraterone prodrug is abiraterone decanoate, or a pharmaceutically acceptable salt thereof,

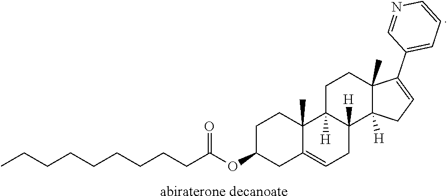

74. The method of claim 72 or 73, wherein the cancer is prostate cancer.

75. The method of any of claims 59-74, wherein the pharmaceutical composition is as described in any of claims 18-30.

76. The method of any of claims 59-75, wherein the pharmaceutical composition is administered to the human subject through an intramuscular injection.

77. The method of any of claims 59-76, wherein the pharmaceutical composition is administered to the human subject once every 1-3 months, such as once every three months.

78. The method of any of claims 59-77, wherein each administration of the pharmaceutical composition comprises administering to the human subject about 50 mg to about 2000 mg of abiraterone decanoate, such as about 180 mg, about 360 mg, about 720 mg, about 1260 mg, about 1800 mg, or any range between the recited values, preferably, the pharmaceutical composition is administered to the human subject once every three months and each administration of the pharmaceutical composition comprises administering to the human subject about 1260 mg of abiraterone decanoate.

79. The method of any of claims 59-78, wherein the human subject is castrated.

80. The method of any one of claims 59-79, wherein the human subject is treated with a gonadotropin-releasing hormone agonist and/or antagonist.

81. The method of any of claims 59-78, wherein the human subject is non-castrated.

82. The method of any of claims 59-81, wherein (i) the human subject is chemotherapy naïve or hormone therapy naïve prior to being administered the pharmaceutical composition, such as a chemotherapy naïve mCRPC patient; and/or (ii) the human subject suffers from hepatic impairment, such as moderate to severe hepatic impairment (Child-Pugh Class B or C), prior to being administered the pharmaceutical composition.

83. The method of any of claims 59-82, wherein (i) the human subject has prostate cancer, and the method does not increase the level of progesterone in the human subject to a level associated with poor clinical outcomes and drug resistance, such as a serum or plasma progesterone level of greater than about 3 nM when measured at 4 weeks, 6 weeks, or 12 weeks after the first administration of the pharmaceutical composition; and/or (ii) the human subject has prostate cancer, and the human subject is characterized as having a serum or plasma progesterone level of greater than about 3 nM after three months of an abiraterone treatment, such as Zytiga (oral abiraterone acetate and prednisone) treatment, prior to the first administration of the pharmaceutical composition.

84. The method of any one of claims 65-83, wherein the human subject suffers from one or more side effects associated with inhibition of CYP17A1 hydroxylase activity or the human subject is susceptible to one or more side effects associated with inhibition of CYP17A1 hydroxylase activity, such as mineralcorticoid toxicity.

85. The method of any one of claims 59-84, wherein the abiraterone prodrug is abiraterone decanoate, and at least a portion of the administered abiraterone decanoate is absorbed through the lymphatic system.

86. The method of any of claims 59-85, further comprising administering to the human subject one or more additional therapy according to any of claims 37-49.

87. The method of any of claims 1-86, wherein the abiraterone prodrug is administered in an effective amount to reduce the serum testosterone level in the human subject to 90% below baseline or lower, when measured at 24 weeks after the first administration of the abiraterone prodrug.

88. The method of any of claims 1-87, wherein the abiraterone prodrug is administered in an effective amount to reduce the serum testosterone level in the human subject to about 50 ng/dL or below (e.g., about 40 ng/dL or below, about 30 ng/dL or below, about 20 ng/dL or below, about 10 ng/dL or below, etc.), when the human subject is a non-castrated human subject, or about 1 ng/dL or below, when the human subject is a castrated human subject, when measured at 24 weeks after the first administration of the abiraterone prodrug.

89. The method of any of claims 1-88, wherein the human subject would benefit from selective inhibition of CYP17A1 lyase activity over CYP17A1 hydroxylase activity.

90. The method of any of claims 1-40 and 42-89, as applicable, wherein the administering of the pharmaceutical composition does not cause a mineralocorticoid toxicity in the human subject.

91. The method of any of any of claims 1-40 and 42-90, as applicable, wherein the human subject is not treated with an agent effective for treating a mineralocorticoid toxicity.

92. The method of any of claims 1-40 and 42-90, as applicable, wherein the human subject is not treated with an agent that is a glucocorticoid or a mineralocorticoid receptor antagonist.

93. A pharmaceutical composition comprising an abiraterone decanoate solution, wherein each milliliter of the abiraterone decanoate solution comprises: (a) abiraterone decanoate in its basic form, in an amount of about 100 mg to about 300 mg (e.g., about 100 mg, about 120 mg, about 150 mg, about 180 mg, about 200 mg or about 250 mg); (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 20 mg (e.g., about 0.5 mg, about 1 mg, about 2 mg, or about 5 mg); and (e) corn oil, q.s. to 1 milliliter, wherein the abiraterone decanoate has the structure of:

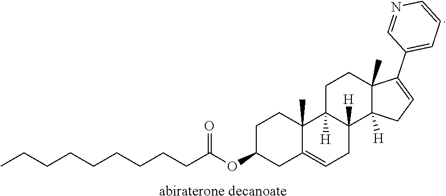

94. The pharmaceutical composition of claim 93, wherein each milliliter of the abiraterone decanoate solution comprises: (a) abiraterone decanoate in its basic form, in an amount of about 180 mg; (b) benzyl alcohol in an amount of about 50 mg to about 150 mg (e.g., about 75 mg, about 100 mg, or about 125 mg); (c) benzyl benzoate in an amount of about 100 mg to about 300 mg (e.g., about 100 mg, about 150 mg, about 200 mg, or about 250 mg); (d) 3-mercapto-1,2-propanediol in an amount of about 0.5 mg to about 2 mg (e.g., about 0.5 mg, about 1 mg, or about 2 mg); and (e) corn oil, q.s. to 1 milliliter.

95. The pharmaceutical composition of claim 93 or 94, wherein the weight ratio of benzyl alcohol to benzyl benzoate in the pharmaceutical composition ranges from about 2:1 to about 1:5 (e.g., about 1:1 to 1:3, such as about 1:2).

96. The method of any of claims 1-92, wherein the pharmaceutical composition is any of those according to claims 93-95.

97. The method of any of claims 1-90 and 96, comprising administering to the human subject (i) abiraterone decanoate intramuscularly once every one to three months, preferably, about 1260 mg of abiraterone decanoate once every three months; and (ii) dexamethasone orally once daily, preferably, at a daily dose of about 0.5 mg/day.
